(19) United States
(12) Patent Application Publication
(10) Pub. No.: US 2026/0293864 A1
(43) Pub. Date: Oct. 1, 2026

(54) MOUSE MODEL WITH HUMANIZED TELOMERE

(71) Applicant: WASHINGTON STATE UNIVESITY, Pullman, WA (US)

(72) Inventors: Jiyue ZHU, Pullman, WA (US); Shuwen WANG, Pullman, WA (US); Fan ZHANG, Pullman, WA (US); De CHENG, Pullman, WA (US)

(21) Appl. No.: 19/480,553

(22) PCT Filed: May 1, 2024
(86) PCT No.: PCT/US2024/027191

§ 371 Date: Oct. 31, 2025

Related U.S. Application Data
(60) Provisional application No. 63/499,465, filed on May 1, 2023.

(51) Int. Cl. A01K 67/0278

(52) U.S. Cl. CPC A01K 67/0278; A01K 2217/052; A01K 2227/105; A01K 2267/03

(57) ABSTRACT

The present disclosure provides a transgenic mouse as well as associated methods thereof for use as a mouse model. The transgenic mouse described herein comprises a humanized mouse telomerase (hmTert) gene contained in a background murine germline that provides shortened telomeres that can replicate humans and various human disease states.

### CROSS-REFERENCE TO RELATED APPLICATIONS

This application claims the benefit under 35 U.S.C. § 119(e) of U.S. Provisional Application Ser. No. 63/499,465, filed on May 1, 2023, the entire disclosure of which is incorporated herein by reference.

### GOVERNMENT RIGHTS

This invention was made with government support under grant numbers R01 GM071725, R21 OD021432, R56 AG073423, and R01 AG073423 awarded by National Institutes of Health. The government has certain rights in the invention.

### BACKGROUND AND SUMMARY

Telomeres play key roles in the development of many human diseases, especially age-related disorders and cancer. Generally, telomeres are replenished by telomerase, a ribonucleoprotein complex containing TERT, TERC, and accessory proteins. Humans are born with 10-15 kb telomeres and adults have 5-15 kb of telomeres. Telomerase activity is very low or undetectable in most adult human tissues. As a result, telomeres are progressively shortened upon successive cell divisions and become exhausted in aged somatic tissues, triggering replicative senescence. Thus, telomeres can be observed to functioning as an aging clock in humans. Furthermore, mutations in human telomerase genes lead to dyskeratosis congenita, a prototypical telomere biology disorder that presents as a multi-system syndrome with a broad spectrum of clinical manifestations, including aplastic anemia and cancer. Short telomere-induced replicative senescence in human cells also functions as a tumor-suppressing mechanism.

Important differences exist in telomerase regulation and telomere length among different mammalian species. In humans, telomerase expression is restricted to a small number of organs (e.g., testis, ovary, and thymus) and telomeres are exhausted in many aged tissues. In contrast, a comparative study of over 60 mammals showed that many mammals, including mice, do not have telomere-mediated replicative aging.

Telomerase expression in mice is less restricted, with most tissues expressing significant levels of Tert mRNA and telomerase activity. Laboratory inbred strains, such as C57BL/6, have long telomeres. Telomerase-null mice (Tert or Terc-KO) can survive up to six generations with no discernible phenotypes in early generations, indicating that mice have adequate telomere reserves that are not exhausted for multiple generations without telomerase.

Mouse models of human diseases have become a central part of biomedical research. Laboratory mice provide the most experimentally accessible mammalian models that share genes, organs, and systemic physiology with humans. However, many mouse models fail to mimic human disease progression, posing translational challenges and limiting their use for human disease research. This facet may contribute to the high failure rates of human clinical trials, particularly in oncology, predicating the need for improved preclinical data from animal models. Therefore, there exists a need for new mouse models for evaluating human diseases and treatments thereof. In particular, mouse models that can be utilized for testing treatments of age-related disorders and cancers are very desirable.

Accordingly, the present disclosure provides a transgenic mouse and associated methods thereof for use as a mouse model. The transgenic mouse described herein comprises a humanized mouse telomerase (hmTert) gene contained in a background murine germline that provides shortened telomeres that can replicate humans and various human disease states.

The transgenic mice described herein comprise a humanized mTert allele (hmTert) that can provide human-like telomere homeostasis. Tert mRNA expression and telomerase activity in these mice were distinct from those in wildtype mice but remarkably similar to those in humans. Importantly, the hmTert gene was able to rescue telomere deficiency when it was crossed into the fifth generation of mTert knockout (KO) mice. The introduction of hmTert allele led to a recalibration of telomere equilibrium length in these mice, providing shorter telomere length compared to wildtype mice but resembling human telomere lengths.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTIONS OF THE DRAWINGS

The detailed description particularly refers to the accompanying figures in which:

FIGS. 1A-1D show the hmTert gene and its expression in mice. FIG. 1A shows the genomic maps of hTERT, mTert, and hmTert loci. Arrows indicate the directions of transcription. Vertical lines are exons; black and dark grey regions represent repetitive sequences, TEs, and VNTRs, respectively. Human and mouse 5′IR and introns 2 & 6 are labeled in blue and red, respectively. FIG. 1B shows telomerase expression in tissues from Tert^{+/-} and Tert^{h/-} littermates. Telomerase activities were determined by TRAP assay. 0.5 μg protein extracts from 4-month-old mice were used except for thymus (0.12 g). The symbols +, h, and − refer to mTert, hmTert, and mTert-KO alleles, respectively. FIG. 1C shows the expression of Tert mRNAs in adult mice. Tissues were collected from 4-month-old Tert^{h/+} mice. hmTert and mTert mRNAs were distinguished by using primers overlapping with the silent mutations in exon 2 of the hmTert allele. Mean and standard deviations (SDs) are shown. ***, p<0.001, two tailed student's t test. FIG. 1D shows hTERT mRNA expression in human tissues. Tert/TERT mRNA data were determined by qRT-PCR assay, normalized to 18S rRNA, and compared to those in Tert^{h/+} ESCs or human ESC HI cells (1.0).

FIGS. 2A-2C show the developmental expression of the hmTert gene in mouse tissues. Where FIG. 2A shows the expression of hmTert and mTert genes during post-natal development of Tert^{h/+} mice. FIG. 2B shows the hmTert and mTert expression during T cell activation. CD4⁺ and CD8⁺ T cells isolated from spleens of Tert^{h/+} mice were co-stimulated with CD3/CD28 antibodies and total RNAs were isolated. Tert mRNA data were determined by qRT-PCR assay, normalized to 18S rRNA, and compared to those in Tert^{h/+} ESCs (1.0). FIG. 2C shows T cell proliferation. Resting T cells were stimulated with CD3/CD28 antibodies for 48-96 h and incubated with 10 μM EdU for 1 h. Percentages of labeled cells were determined by flow cytometry.

FIGS. 3A-3J show the functions of the hmTert gene in mice. FIG. 3A shows the breeding strategy. Telomere length of splenocytes from 2-month-old Tert^{+/-}, Tert^{h/-}, and Tert^{-/-} mice were determined by Flow-FISH, as shown in FIG. 3B and in FIG. 3C, the telomere restriction fragment (TRF) analysis is shown. Where FIG. 3B shows telomere Fow-FISH. Telomere signals were detected by hybridization to FAM-(CCCTAA)₃ oligonucleotide. Fluorescence signals were compared to that of wildtype C57BL/6J mice (1.0). FIG. 3C shows TRF analysis. Splenocyte genomic DNAs were digested with HinfI and RsaI, followed by pulsed-field gel electrophoresis and Southern blotting. Positions of size markers are shown on the left (kb). FIG. 3D shows the litter sizes of breeding between Tert^{+/-} and Tert^{-/-} (red), Tert^{h/-} and Tert^{-/-} (blue), Tert^{-/-} and Tert^{-/-} (black) mice. FIG. 3E shows the body weight of male (upper) and female (lower) mice at 8-week of age. FIG. 3F shows the testis weight of mice at 10-15-week age. FIG. 3G shows the H&E staining of seminiferous tubules in testes from Tert^{+/-}, Tert^{h/-}, and Tert^{-/-} mice. Yellow arrowheads indicate aberrant tubules. FIG. 3H shows the average percentages of aberrant seminiferous tubules in testes from 3-5 mice in each group. FIG. 3I and FIG. 3J shows the survival curves of mice with mTert, hmTert, and mTert-KO alleles. Mice were bred as shown in panel A. Kaplan-Meier survival curves of G4, shown in FIG. 21 and G5, the mice are shown in FIG. 3J. P-values of survival curve comparisons were calculated using logrank test. Means and SDs are shown.

FIGS. 4A-4E shows a comparison of G6 mice with mTert and hmTert alleles. FIG. 4A shows the mouse breeding scheme. FIG. 4B shows TRF analysis of representative animals. Splenocyte genomic DNAs were digested with HinfI and RsaI, followed by pulsed-field gel electrophoresis and Southern blotting. FIG. 4C shows Kaplan-Meier survival curves of the mice. P-values comparing indicated paired curves were determined using logrank tests. FIG. 4D shows the body weight of male (upper) and female (lower) mice at 8-week of age. FIG. 4E shows the testis weight of mice at 10-15-week age. FIG. 4F shows whole blood cell counts in adult mice of 3-6 months by hematology analyses. FIG. 4G shows lymphocyte counts in peripheral blood. Cells were stained using antibodies and analyzed by flow cytometry. Means and SDs are shown. ns, not significant; **, P<0.01; ***, P<0.001; ****, P<0.0001; one-way Anova. FIG. 4H shows the histopathology of small intestines of adult mice of 8-10 months. The bar indicates 100 m. FIG. 4I shows the expression of genes regulating cellular senescence and proliferation in small intestine. Each column represents an individual mouse. Relative mRNA levels were determined by qRT-PCR and normalized to 18S rRNA. Means and SDs are shown.

FIGS. 5A-5C show telomere length and genotype ratios of G5 and G6 mice in from FIG. 4. FIG. 5A and FIG. 5B show telomere length as determined by Fow-FISH. Telomere fluorescent signals of representative mice are shown in FIG. 5A and the data are summarized in FIG. 5B. FIG. 5C shows the genotype ratios of born offspring from G5 parents. Each data point represents one litter.

FIGS. 6A-6B show hematopoietic cells in adult mice of 3-6 months. Mice were bred FIG. 4. FIG. 6A shows lymphocyte counts in spleen and FIG. 6B, the lymphocyte counts in bone marrow. Cells were stained using antibodies and analyzed by flow cytometry. Each data point represent one animal. Means and SDs are shown. ns, not significant; *, P<0.05; **, P<0.01; ***, P<0.001; ****, P<0.0001; one-way Anova.

FIGS. 7A-7H show telomere length homeostasis in mice during Tert^{h/-} intercrosses. FIG. 7A shows the breeding strategy. Tert^{h/-} progeny from G4 Tert^{h/-} parents were intercrossed. FIG. 7B shows telomere length as determined by Flow-FISH. Splenocytes from 2-month-old mice were used for the analyses. FIG. 7C shows the body weight of 8-week-old male and female mice. FIG. 7D shows litter sizes. FIG. 7E shows the testis weight of mice at 10-15-week age. FIG. 7F shows the Flow-FISH comparing telomere lengths of Tert^{h/h}, Tert^{h/-}, and Tert^{-/-} littermates. FIG. 7G shows the TRF analysis. Splenocyte genomic DNAs were digested with HinfI and RsaI, followed by 0.6% Agraose gel electrophoresis and Southern blotting. Sizes are indicated on the left (kb). MW, molecular weight marker. NHF (P11), passage 11 normal human foreskin fibroblasts. FIG. 7H shows the genotype ratios of progeny in intercrosses at 7, 21, and 56 postnatal days. Means and SDs are shown in FIGS. 7B-7F.

FIGS. 8A-8F show telomere length homeostasis during incrosses of Tert^{h/h} mice. FIG. 8A shows the breeding schemes. Tert^{h/h} progeny from G4, G4.8, and G4.14 Tert^{h/-} parents were successively incrossed. FIG. 8B shows the relative telomere signals. Telomere signals were determined by Flow-FISH and normalized to that of wildtype C57BL/6J mice (50 kb). FIG. 8C shows TRF analysis. FIG. 8D shows body weight. FIG. 8E shows litter sizes. FIG. 8F shows testis weight. Each data point represents one animal. Means and SDs are shown.

FIGS. 9A-9B show blood cell counts of Tert^{h/h} mice. FIG. 9A shows whole blood counts. FIG. 9B shows white blood cell counts in peripheral blood. Cells were stained using antibodies and analyzed by flow cytometry. Each data point represents one animal. Means and SDs are shown.

FIGS. 10A-10E show a comparison of mTert and hmTert alleles. FIG. 10A shows the breeding strategy. G6 Tert^{+/-} and Tert^{h/-} mice from FIG. 4A were independently intercrossed. FIG. 10B shows the relative telomere signals as determined by Pow-FISH and normalized to that of wildtype C57BL/6J mice. FIG. 10C shows body weight. FIG. 10D shows the litter sizes. FIG. 10E shows the testis weight. Each data point represents one animal. Means and SDs are shown.

FIGS. 11A-11E show dextran sulfate sodium (DSS)-induced colitis in mice. FIG. 11A shows the general experimental strategy. 7-8-month-old Tert^{+/+} (wildtype C57BL/6J) and Tert^{h/h} (G4.8h) mice were given drinking water with or without 3% DSS for 6 days, followed by 1 day of pure drinking water. Intraperitoneal EdU injection was performed 2 hours before tissue collection. FIG. 11B shows representative images of colons and spleens following DSS treatment. FIG. 11C shows spleen weight. Spleen weight was normalized to the body weight of each mouse. FIG. 11D shows EdU staining of colon crypt sections. Colon tissues were labeled with anti-EdU (white) and E-cadherin (green) antibodies, as well as Hoechst dye for nuclear staining (blue). Images were captured using a Zeiss Image M2 microscope. Representative images are shown. FIG. 11E shows quantification of EdU-positive cells. Each data point represents the average number of EdU-positive cells per colon crypt in 30 crypts from one animal. ns, not significant; *, P<0.05; **, P<0.01; ****, P<0.0001; N=6; two-way Anova.

FIG. 12 shows the sequence of SEQ ID NO:1.

FIG. 12 shows the sequence of SEQ ID NO:2, which is the sequence of the hmTert knock-in locus. The upper case represents mouse sequence while the bold lower case represents human sequence. The 5′IR (5′ intergenic region) is distinctly delineated with an underline, the In2 (intron 2) is highlighted with a double underline, and the In6 (intron 6) is emphasized with a wave underline.

FIG. 14 shows the sequence of SEQ ID NO:3, which displays hmTert with introns 2 and 6.

### DETAILED DESCRIPTION

Various embodiments of the invention are described herein as follows. In an illustrative aspect, a transgenic mouse is provided. The transgenic mouse comprises i) a background murine germline and ii) a humanized mouse telomerase (hmTert) gene contained in the background murine germline.

In an embodiment, the transgenic mouse is a humanized transgenic mouse. In an embodiment, the transgenic mouse is a humanized transgenic mouse. In an embodiment, the hmTert gene comprises SEQ ID NO:1. In an embodiment, the hmTert gene consists essentially of SEQ ID NO:1. In an embodiment, the hmTert gene consists of SEQ ID NO:1.

In an embodiment, the hmTert gene has at least 80% sequence identity to SEQ ID NO: 1. In an embodiment, the hmTert gene has at least 85% sequence identity to SEQ ID NO:1. In an embodiment, the hmTert gene has at least 90% sequence identity to SEQ ID NO:1. In an embodiment, the hmTert gene has at least 95% sequence identity to SEQ ID NO:1. In an embodiment, the hmTert gene has at least 96% sequence identity to SEQ ID NO:1. In an embodiment, the hmTert gene has at least 97% sequence identity to SEQ ID NO:1. In an embodiment, the hmTert gene has at least 98% sequence identity to SEQ ID NO:1. In an embodiment, the hmTert gene has at least 99% sequence identity to SEQ ID NO:1.

In an embodiment, the hmTert gene comprises SEQ ID NO:1, and SEQ ID NO:1 comprises intron 2 of the human TERT gene. In an embodiment, the hmTert gene comprises SEQ ID NO:1, and SEQ ID NO:1 comprises intron 6 of the human TERT gene. In an embodiment, the hmTert gene comprises SEQ ID NO:1, and SEQ ID NO:1 comprises intron 2 and intron 6 from the human TERT gene.

In an embodiment, the hmTert gene comprises SEQ ID NO:1, and SEQ ID NO:1 comprises a 5′ intergenic region (5′IR) of the human TERT gene. In an embodiment, the hmTert gene comprises SEQ ID NO:1, and SEQ ID NO:1 comprises a 5′IR and intron 2 of the human TERT gene. In an embodiment, the hmTert gene comprises SEQ ID NO:1, and SEQ ID NO:1 comprises a 5′IR and intron 6 of the human TERT gene. In an embodiment, the hmTert gene comprises SEQ ID NO:1, and SEQ ID NO:1 comprises a 5′IR and intron 2 and intron 6 of the human TERT gene.

In an embodiment, the hmTert gene comprises SEQ ID NO:2. In an embodiment, the hmTert gene consists essentially of SEQ ID NO:2. In an embodiment, the hmTert gene consists of SEQ ID NO:2.

In an embodiment, the hmTert gene has at least 80% sequence identity to SEQ ID NO:2. In an embodiment, the hmTert gene has at least 85% sequence identity to SEQ ID NO:2. In an embodiment, the hmTert gene has at least 90% sequence identity to SEQ ID NO:2. In an embodiment, the hmTert gene has at least 95% sequence identity to SEQ ID NO:2. In an embodiment, the hmTert gene has at least 96% sequence identity to SEQ ID NO:2. In an embodiment, the hmTert gene has at least 97% sequence identity to SEQ ID NO:2. In an embodiment, the hmTert gene has at least 98% sequence identity to SEQ ID NO:2. In an embodiment, the hmTert gene has at least 99% sequence identity to SEQ ID NO:2.

In an embodiment, the hmTert gene comprises SEQ ID NO:2, and SEQ ID NO:2 comprises intron 2 of the human TERT gene. In an embodiment, the hmTert gene comprises SEQ ID NO:2, and SEQ ID NO:2 comprises intron 6 of the human TERT gene. In an embodiment, the hmTert gene comprises SEQ ID NO:2, and SEQ ID NO:2 comprises intron 2 and intron 6 from the human TERT gene.

In an embodiment, the hmTert gene comprises SEQ ID NO:2, and SEQ ID NO:2 comprises a 5′ intergenic region (5′IR) of the human TERT gene. In an embodiment, the hmTert gene comprises SEQ ID NO:2, and SEQ ID NO:2 comprises a 5′IR and intron 2 of the human TERT gene. In an embodiment, the hmTert gene comprises SEQ ID NO:2, and SEQ ID NO:2 comprises a 5′IR and intron 6 of the human TERT gene. In an embodiment, the hmTert gene comprises SEQ ID NO:2, and SEQ ID NO:2 comprises a 5′IR and intron 2 and intron 6 of the human TERT gene.

In an embodiment, the hmTert gene comprises SEQ ID NO:3. In an embodiment, the hmTert gene consists essentially of SEQ ID NO:3. In an embodiment, the hmTert gene consists of SEQ ID NO:3.

In an embodiment, the hmTert gene has at least 80% sequence identity to SEQ ID NO:3. In an embodiment, the hmTert gene has at least 85% sequence identity to SEQ ID NO:3. In an embodiment, the hmTert gene has at least 90% sequence identity to SEQ ID NO:3. In an embodiment, the hmTert gene has at least 95% sequence identity to SEQ ID NO:3. In an embodiment, the hmTert gene has at least 96% sequence identity to SEQ ID NO:3. In an embodiment, the hmTert gene has at least 97% sequence identity to SEQ ID NO:3. In an embodiment, the hmTert gene has at least 98% sequence identity to SEQ ID NO:3. In an embodiment, the hmTert gene has at least 99% sequence identity to SEQ ID NO:3.

In an embodiment, the hmTert gene comprises SEQ ID NO:3, and SEQ ID NO:3 comprises intron 2 of the human TERT gene. In an embodiment, the hmTert gene comprises SEQ ID NO:3, and SEQ ID NO:3 comprises intron 6 of the human TERT gene. In an embodiment, the hmTert gene comprises SEQ ID NO:3, and SEQ ID NO:3 comprises intron 2 and intron 6 of the human TERT gene.

In an embodiment, the hmTert gene comprises SEQ ID NO:3, and SEQ ID NO:3 comprises a 5′ intergenic region (5′IR) of the human TERT gene. In an embodiment, the hmTert gene comprises SEQ ID NO:3, and SEQ ID NO:3 comprises a 5′IR and intron 2 of the human TERT gene. In an embodiment, the hmTert gene comprises SEQ ID NO:3, and SEQ ID NO:3 comprises a 5′IR and intron 6. In an embodiment, the hmTert gene comprises SEQ ID NO:3, and SEQ ID NO:3 comprises a 5′IR and intron 2 and intron 6 of the human TERT gene.

In an embodiment, the background murine germline is from a species of Mus genus. In an embodiment, the background murine germline is a strain of a Mus musculus line.

In an embodiment, the background murine germline is a substrain of C57BL/6 line. In an embodiment, the background murine germline is a substrain of J:NU. In an embodiment, the background murine germline is a substrain of NU/J. In an embodiment, the background murine germline is a substrain of 129S1/SvImJ. In an embodiment, the background murine germline is a substrain of 129X1/SvJ. In an embodiment, the background murine germline is a substrain of A/J. In an embodiment, the background murine germline is a substrain of AKR/J. In an embodiment, the background murine germline is a substrain of BALB/cByJ. In an embodiment, the background murine germline is a substrain of BALB/cJ. In an embodiment, the background murine germline is a substrain of C3H/HeJ. In an embodiment, the background murine germline is a substrain of C57BL/6J. In an embodiment, the background murine germline is a substrain of C57BL/6NJ. In an embodiment, the background murine germline is a substrain of C57BL/10J. In an embodiment, the background murine germline is a substrain of CBA/J. In an embodiment, the background murine germline is a substrain of CBA/CaJ. In an embodiment, the background murine germline is a substrain of DBA/1J. In an embodiment, the background murine germline is a substrain of DBA/2J. In an embodiment, the background murine germline is a substrain of FVB/NJ. In an embodiment, the background murine germline is a substrain of C57BL/6J-Apc^{Min}/J.

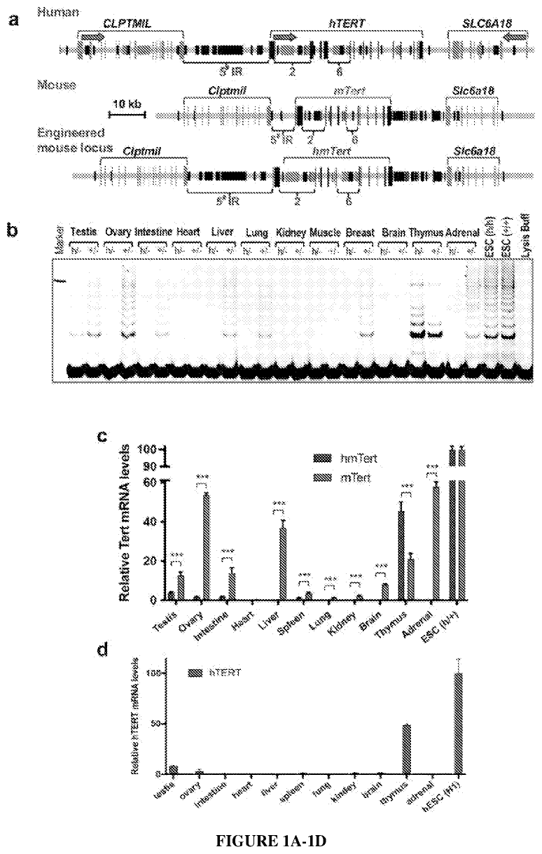

In an embodiment, the transgenic mouse comprises expression maintenance of the hmTert gene. As used herein, expression maintenance refers to the ability of a mouse to continue expressing a particular gene upon one or more subsequent generations of breeding. In an embodiment, the transgenic mouse comprises humanized telomere homeostasis. As used herein, humanized telomere homeostasis can refer to mice possessing telomerase expression that is suppressed in adult tissues (see, e.g., FIG. 1) and/or that the telomeres comprise an average of telomere length from five to twenty (5-20) kilobase (kb). In an embodiment, the telomere homeostasis is maintained upon interbreeding for one or more generations.

In an embodiment, the mouse comprises one or more telomeres, and the telomeres comprise an average telomere length from five to twenty (5-20) kilobase (kb). In an embodiment, the telomeres comprise an average telomere length from 5-20 kb. In an embodiment, the telomeres comprise an average telomere length from 5-15 kb. In an embodiment, the telomeres comprise an average telomere length from 5-10 kb. In an embodiment, the telomeres comprise an average telomere length from 6-20 kb. In an embodiment, the telomeres comprise an average telomere length from 6-18 kb. In an embodiment, the telomeres comprise an average telomere length from 6-16 kb. In an embodiment, the telomeres comprise an average telomere length from 6-14 kb. In an embodiment, the telomeres comprise an average telomere length from 6-12 kb. In an embodiment, the telomeres comprise an average telomere length from 6-10 kb. In an embodiment, the telomeres comprise an average telomere length from 6-8 kb. In an embodiment, the telomeres comprise an average telomere length from 8-20 kb. In an embodiment, the telomeres comprise an average telomere length from 8-18 kb. In an embodiment, the telomeres comprise an average telomere length from 8-16 kb. In an embodiment, the telomeres comprise an average telomere length from 8-14 kb. In an embodiment, the telomeres comprise an average telomere length from 8-12 kb. In an embodiment, the telomeres comprise an average telomere length from 8-10 kb. In an embodiment, the telomeres comprise an average telomere length from 10-20 kb. In an embodiment, the telomeres comprise an average telomere length from 10-18 kb. In an embodiment, the telomeres comprise an average telomere length from 10-16 kb. In an embodiment, the telomeres comprise an average telomere length from 10-14 kb. In an embodiment, the telomeres comprise an average telomere length from 10-12 kb. In an embodiment, the telomeres comprise an average telomere length from 12-20 kb. In an embodiment, the telomeres comprise an average telomere length from 12-18 kb. In an embodiment, the telomeres comprise an average telomere length from 12-16 kb. In an embodiment, the telomeres comprise an average telomere length from 12-14 kb. In an embodiment, the telomeres comprise an average telomere length from 14-20 kb. In an embodiment, the telomeres comprise an average telomere length from 14-18 kb. In an embodiment, the telomeres comprise an average telomere length from 14-16 kb. In an embodiment, the telomeres comprise an average telomere length from 16-20 kb. In an embodiment, the telomeres comprise an average telomere length from 16-18 kb.

In an embodiment, the telomeres comprise an average telomere length less than 20 kb. In an embodiment, the telomeres comprise an average telomere length less than 15 kb. In an embodiment, the telomeres comprise an average telomere length less than 10 kb.

In an illustrative aspect, a method for evaluating a treatment is provided. The method comprises the steps of i) administering the treatment to a transgenic mouse and ii) analyzing a response to the treatment in the transgenic mouse, wherein the response provides evaluation of the treatment.

In an embodiment, the treatment is a drug. In an embodiment, the drug is used to treat aplastic anemia. In an embodiment, the drug is used to treat idiopathic aplastic anemia. In an embodiment, the drug is used to treat pulmonary fibrosis. In an embodiment, the drug is used to treat familial idiopathic pulmonary fibrosis. In an embodiment, the drug is used to treat dyskeratosis cogenita. In an embodiment, the drug is used to treat hepatic disease. In an embodiment, the drug is used to treat cirrhosis with inflammation. In an embodiment, the drug is used to treat nodular regenerative hyperplasia.

In an embodiment, the drug is used to treat cancer. In an embodiment, the cancer is carcinoma with nonreciprocal translocations. In an embodiment, the cancer is colorectal cancer. In an embodiment, the cancer is esophageal cancer. In an embodiment, the cancer is head and neck squamous cell carcinoma. In an embodiment, the cancer is skin cancer. In an embodiment, the cancer is anorectal cancer. In an embodiment, the cancer is acute myeloid leukemia. In an embodiment, the cancer is leukemic transformation in myelodysplasia. In an embodiment, the cancer is secondary myelodysplasia. In an embodiment, the cancer is secondary leukemia. In an embodiment, the cancer is basal cell cancer of the skin. In an embodiment, the cancer is cancer of the lung. In an embodiment, the cancer is cancer of the bladder. In an embodiment, the cancer is cancer of the prostate. In an embodiment, the cancer is cancer of the cervix. In an embodiment, the cancer is glioblastoma. In an embodiment, the cancer is renal cell carcinoma. In an embodiment, the cancer is Barrett's esophagus hepatocellular carcinoma.

In an embodiment, the drug is used to treat inflammatory bowel disease. In an embodiment, the drug is used to treat chronic hepatitis B infection. In an embodiment, the drug is used to treat ulcerative colitis. In an embodiment, the drug is used to treat chronic graft-versus-host disease. In an embodiment, the drug is used to treat cardiovascular disease.

In an embodiment, the treatment is radiation. In an embodiment, the treatment is surgery. In an embodiment, the treatment is a transplant. In an embodiment, the treatment is immunotherapy. In an embodiment, the treatment is gene therapy. In an embodiment, the treatment is biological engineering.

In an embodiment, the treatment is a disease-causing agent. In an embodiment, the disease-causing agent is a pathogen. In an embodiment, the disease-causing agent is a carcinogen. In an embodiment, the disease-causing agent is a cancer cell.

In an embodiment, the administering comprises an injection. In an embodiment, the administering comprises oral administration. In an embodiment, the administering comprises topical application. In an embodiment, the administering comprises inhalation. In an embodiment, the administering comprises radiation. In an embodiment, the administering comprises transplantation.

In an embodiment, the response is an immune system response. In an embodiment, the response is tumorigenesis. In an embodiment, the response is senescence. In an embodiment, the response is cellular aging. In an embodiment, the response is a compromised innate immune response. In an embodiment, the response is a compromised adaptive immune response. In an embodiment, the response is apoptosis. In an embodiment, the response is oncogenic transformation. In an embodiment, the response is side effects. In an embodiment, the response is presence of disease. In an embodiment, the response is telomere length. In an embodiment, the response is telomerase activity. In an embodiment, the response is cellular metabolic activity. In an embodiment, the response is gene expression. In an embodiment, the response is RNA expression. In an embodiment, the response is protein expression.

In an embodiment, the response is to cancer. In an embodiment, the cancer is melanoma. In an embodiment, the cancer is adrenal gland cancer. In an embodiment, the cancer is bladder cancer. In an embodiment, the cancer is bone cancer. In an embodiment, the cancer is blood cancer. In an embodiment, the cancer is brain cancer. In an embodiment, the cancer is breast cancer. In an embodiment, the cancer is cervical cancer. In an embodiment, the cancer is colon cancer. In an embodiment, the cancer is endometrium cancer. In an embodiment, the cancer is esophageal cancer. In an embodiment, the cancer is eye cancer. In an embodiment, the cancer is head and neck cancer. In an embodiment, the cancer is intestinal cancer. In an embodiment, the cancer is kidney cancer. In an embodiment, the cancer is liver cancer. In an embodiment, the cancer is lung cancer. In an embodiment, the cancer is mouth cancer. In an embodiment, the cancer is muscle cancer. In an embodiment, the cancer is nose cancer. In an embodiment, the cancer is ovarian cancer. In an embodiment, the cancer is pancreatic cancer. In an embodiment, the cancer is pituitary cancer. In an embodiment, the cancer is prostate cancer. In an embodiment, the cancer is skin cancer. In an embodiment, the cancer is stomach cancer. In an embodiment, the cancer is thymus cancer. In an embodiment, the cancer is thyroid cancer. In an embodiment, the cancer is testicular cancer. In an embodiment, the cancer is an immune system cancer. In an embodiment, the cancer is a nervous system cancer. In an embodiment, the cancer is a neuroendocrine system cancer. In an embodiment, the cancer is lymphoproliferative disease. In an embodiment, the cancer is a hematologic malignancy. In an embodiment, the cancer is a myelodysplastic syndrome. In an embodiment, the cancer is acute myeloid leukemia.

In an embodiment, the response is to a rejuvenation or lifespan extension. In an embodiment, the rejuvenation or lifespan extension is a telomerase-mediated rejuvenation. In an embodiment, the rejuvenation or lifespan extension is epigenetic reprogramming. In an embodiment, the rejuvenation or lifespan extension is to a senolytic therapy. In an embodiment, the rejuvenation or lifespan extension is to an immunotherapy.

In an embodiment, the response is skin appearance. In an embodiment, the response is hair loss/growth. In an embodiment, the response is nail aging/rejuvenation. In an embodiment, the response is to skincare products. In an embodiment, the response is to cosmetics. In an embodiment, the response is to hair graying. In an embodiment, the response is to alopecia. In an embodiment, the response is to hair loss treatment. In an embodiment, the response is to hyperpigmentation. In an embodiment, the response is to hypopigmentation. In an embodiment, the response is to abnormal skin pigmentation. In an embodiment, the response is to nail dystrophy. In an embodiment, the response is to adermatoglyphia.

In an embodiment, the response is to shortened telomeres. In an embodiment, the shortened telomeres result from Coats Plus syndrome. In an embodiment, the shortened telomeres result from Fanconi anemia. In an embodiment, the shortened telomeres result from Revesz syndrome. In an embodiment, the shortened telomeres result from Hoyeraal-Hreidarsson syndrome.

In an embodiment, the response is to telomere extension. In an embodiment, the telomere extension results from telomerase activation. In an embodiment, the telomere extension results from epigenetic reprogramming.

In an embodiment, the response is to stem cell proliferation and maintenance. In an embodiment, the stem cell proliferation and maintenance comprises telomere-mediated stem cell failures manifested in lesions of skin, hematopoietic, gastrointestinal, and reproductive systems.

In an embodiment, the response is to an immunologic system reaction. In an embodiment, the response is to a pharmaceutical event. In an embodiment, the response is to an environmental exposure. In an embodiment, the response is to a human lifestyle event.

In an embodiment, the response is to a disease. In an embodiment, the disease is an aging or age-related disease. In an embodiment, the aging or age-related disease is senescence/aging. In an embodiment, the aging or age-related disease is longevity. In an embodiment, the aging or age-related disease is Dyskeratosis congenita (DC). In an embodiment, the aging or age-related disease is Werner syndrome. In an embodiment, the aging or age-related disease is Bloom syndrome. In an embodiment, the aging or age-related disease is Hutchinson-Gilford progeria syndrome.

In an embodiment, the disease is a digestive system disease. In an embodiment, the digestive system disease is oral leukoplakia. In an embodiment, the digestive system disease is oesophageal structures and webs. In an embodiment, the digestive system disease is telangiectasias prone to hemorrhage. In an embodiment, the digestive system disease is villus atrophy. In an embodiment, the digestive system disease is enteropathy.

In an embodiment, the disease is a lung disease. In an embodiment, the lung disease is pulmonary fibrosis (PF). In an embodiment, the lung disease is chronic obstructive pulmonary disease (COPD). In an embodiment, the lung disease is emphysema. In an embodiment, the lung disease is non-cystic fibrosis bronchiectasis.

In an embodiment, the disease is a neurodegenerative disease. In an embodiment, the neurodegenerative disease is dementia. In an embodiment, the neurodegenerative disease is Alzheimer's disease (AD). In an embodiment, the neurodegenerative disease is Parkinson's disease (PD). In an embodiment, the neurodegenerative disease is Huntington's disease (HD).

In an embodiment, the disease is an eye disease. In an embodiment, the eye disease is age-related macular degeneration.

In an embodiment, the disease is a bone marrow disease. In an embodiment, the bone marrow disease is aplastic anemia. In an embodiment, the bone marrow disease is bone marrow failure. In an embodiment, the bone marrow disease is myelodysplastic syndrome (MDS).

In an embodiment, the disease is an immune system reaction. In an embodiment, the immune system reaction is an innate immune reaction. In an embodiment, the immune system reaction is an adaptive immune reaction. In an embodiment, the immune system reaction is immune cell senescence and exhaustion. In an embodiment, the immune system reaction is a B cell lymphopenia/immunodeficiency. In an embodiment, the immune system reaction is a T cell lymphopenia/immunodeficiency. In an embodiment, the immune system reaction is a natural killer (NK) cell lymphopenia/immunodeficiency.

In an embodiment, the disease is an infectious disease. In an embodiment, the infectious disease is caused by HIV. In an embodiment, the infectious disease is caused by SARS-CoV-2. In an embodiment, the infectious disease is caused by a hepatitis virus.

In an embodiment, the disease is an inflammatory disease. In an embodiment, the inflammatory disease is inflammatory bowel disease (IBD). In an embodiment, the inflammatory disease is Crohn's disease (CD). In an embodiment, the inflammatory disease is ulcerative colitis (UC). In an embodiment, the inflammatory disease is rheumatoid arthritis. In an embodiment, the inflammatory disease is atherosclerosis. In an embodiment, the inflammatory disease is a chronic renal disease.

In an embodiment, the disease is a metabolic disease. In an embodiment, the metabolic disease is chronic inflammation. In an embodiment, the metabolic disease is obesity. In an embodiment, the metabolic disease is dyslipidaemia. In an embodiment, the metabolic disease is hypertension. In an embodiment, the metabolic disease is insulin resistance. In an embodiment, the metabolic disease is diabetes.

In an embodiment, the disease is a hepatic disease. In an embodiment, the hepatic disease is cirrhosis. In an embodiment, the hepatic disease is non-alcoholic fatty liver disease (NAFLD). In an embodiment, the hepatic disease is non-alcoholic steatohepatitis (NASH). In an embodiment, the hepatic disease is fibrosis. In an embodiment, the hepatic disease is primary biliary cirrhosis. In an embodiment, the hepatic disease is alcoholic liver disease. In an embodiment, the hepatic disease is nodular regenerative hyperplasia hepatopulmonary syndrome. In an embodiment, the hepatic disease is chronic viral hepatitis.

In an embodiment, the disease is a cardiovascular disease. In an embodiment, the cardiovascular disease is cardiomyocyte hypertrophy. In an embodiment, the cardiovascular disease is fibrosis. In an embodiment, the cardiovascular disease is cardiac ischaemia-reperfusion injury (IRI). In an embodiment, the cardiovascular disease is hypertrophic or dilated cardiomyopathy. In an embodiment, the cardiovascular disease is myocardial infarction. In an embodiment, the cardiovascular disease is atherosclerosis.

In an embodiment, the disease is a musculoskeletal disease. In an embodiment, the musculoskeletal disease is osteopenia. In an embodiment, the musculoskeletal disease is osteoarthritis. In an embodiment, the musculoskeletal disease is rheumatoid arthritis. In an embodiment, the musculoskeletal disease is osteoporosis. In an embodiment, the musculoskeletal disease is skeletal fragility. In an embodiment, the musculoskeletal disease is avascular necrosis. In an embodiment, the musculoskeletal disease is Duchenne muscular dystrophy (DMD).

In an embodiment, the disease is a kidney disease. In an embodiment, the kidney disease is an acute kidney injury. In an embodiment, the kidney disease is glomerulonephritis. In an embodiment, the kidney disease is diabetic nephropathy. In an embodiment, the kidney disease is polycystic kidney disease. In an embodiment, the kidney disease is kidney fibrosis. In an embodiment, the kidney disease is chronic kidney disease (CKD).

In an embodiment, the disease is a reproductive disease. In an embodiment, the reproductive disease comprises testicular function. In an embodiment, the reproductive disease comprises ovarian function. In an embodiment, the reproductive disease comprises male infertility. In an embodiment, the reproductive disease comprises female infertility.

In an embodiment, the response is a susceptibility and resiliency of human health in response to a pharmaceutical intervention. In an embodiment, the pharmaceutical intervention is rapamycin. In an embodiment, the pharmaceutical intervention is 17β-oestradial. In an embodiment, the pharmaceutical intervention is senolytic strategies. In an embodiment, the pharmaceutical intervention is drug efficacy. In an embodiment, the pharmaceutical intervention is drug toxicity. In an embodiment, the pharmaceutical intervention is a drug-drug interaction. In an embodiment, the pharmaceutical intervention is a pharmacodynamic study.

In an embodiment, the response is susceptibility and resiliency of human health in response to an environmental exposure. In an embodiment, the environmental exposure is air pollution. In an embodiment, the environmental exposure is food poisoning. In an embodiment, the environmental exposure is heavy metal. In an embodiment, the environmental exposure is hazardous working condition. In an embodiment, the environmental exposure is algae. In an embodiment, the environmental exposure is water pollution. In an embodiment, the environmental exposure is a mutagen. In an embodiment, the environmental exposure is radiation. In an embodiment, the environmental exposure is space travel.

In an embodiment, the response is susceptibility and resiliency of human health in response to a lifestyle change. In an embodiment, the lifestyle change is dietary restriction. In an embodiment, the lifestyle change is cigarette smoking. In an embodiment, the lifestyle change is exercise. In an embodiment, the lifestyle change is physical work. In an embodiment, the lifestyle change is sleep deprivation. In an embodiment, the lifestyle change is night shift working condition. In an embodiment, the lifestyle change is chronic fatigue. In an embodiment, the lifestyle change is life stress. In an embodiment, the lifestyle change is psychological stress. In an embodiment, the lifestyle change is a psychological disorder. In an embodiment, the lifestyle change is mental health. In an embodiment, the lifestyle change is a psychiatric illness.

In an illustrative aspect, a method of generating a transgenic mouse is provided. The method comprises the steps of inserting an hmTert gene into a background murine germline to form a modified embryonic stem cell; and generating a first-generation mouse from the modified embryonic stem cell, wherein the first-generation mouse comprises the hmTert gene.

In an embodiment, the method further comprises breeding one or more generations from the first-generation mouse to generate a progeny mouse. In an embodiment, the breeding one or more generations comprises controlled breeding with one or more of hmTert, mTert, and mTert-knockout strains. In an embodiment, the progeny mouse exhibits average telomere length of five to fifteen (5-15) kilobase.

In an illustrative aspect, a method of generating a transgenic mouse is provided. The method comprises a step of breeding a first mouse comprising an hmTert gene with a second mouse to generate a heterozygous mouse.

In an embodiment, the method further comprises a step of breeding one or more generations from the heterozygous mouse.

The following numbered embodiments are contemplated and are non-limiting:
1. A transgenic mouse comprising i) a background murine germline and ii) a humanized mouse telomerase (hmTert) gene contained in the background murine germline.
2. The transgenic mouse of clause 1, any other suitable clause, or any combination of suitable clauses, wherein the transgenic mouse is a humanized transgenic mouse.
3. The transgenic mouse of clause 1, any other suitable clause, or any combination of suitable clauses, wherein the hmTert gene comprises SEQ ID NO:2.
4. The transgenic mouse of clause 1, any other suitable clause, or any combination of suitable clauses, wherein the hmTert gene consists essentially of SEQ ID NO:2.
5. The transgenic mouse of clause 1, any other suitable clause, or any combination of suitable clauses, wherein the hmTert gene consists of SEQ ID NO:2.
6. The transgenic mouse of clause 1, any other suitable clause, or any combination of suitable clauses, wherein the hmTert gene has at least 80% sequence identity to SEQ ID NO:2.
7. The transgenic mouse of clause 1, any other suitable clause, or any combination of suitable clauses, wherein the hmTert gene has at least 85% sequence identity to SEQ ID NO:2.
8. The transgenic mouse of clause 1, any other suitable clause, or any combination of suitable clauses, wherein the hmTert gene has at least 90% sequence identity to SEQ ID NO:2.
9. The transgenic mouse of clause 1, any other suitable clause, or any combination of suitable clauses, wherein the hmTert gene has at least 95% sequence identity to SEQ ID NO:2.
10. The transgenic mouse of clause 1, any other suitable clause, or any combination of suitable clauses, wherein the hmTert gene has at least 96% sequence identity to SEQ ID NO:2.
11. The transgenic mouse of clause 1, any other suitable clause, or any combination of suitable clauses, wherein the hmTert gene has at least 97% sequence identity to SEQ ID NO:2.
12. The transgenic mouse of clause 1, any other suitable clause, or any combination of suitable clauses, wherein the hmTert gene has at least 98% sequence identity to SEQ ID NO:2.
13. The transgenic mouse of clause 1, any other suitable clause, or any combination of suitable clauses, wherein the hmTert gene has at least 99% sequence identity to SEQ ID NO:2.
14. The transgenic mouse of clause 1, any other suitable clause, or any combination of suitable clauses, wherein the hmTert gene comprises SEQ ID NO:2, and wherein SEQ ID NO:2 comprises intron 2.
15. The transgenic mouse of clause 1, any other suitable clause, or any combination of suitable clauses, wherein the hmTert gene comprises SEQ ID NO:2, and wherein SEQ ID NO:2 comprises intron 6.
16. The transgenic mouse of clause 1, any other suitable clause, or any combination of suitable clauses, wherein the hmTert gene comprises SEQ ID NO:2, and wherein SEQ ID NO:2 comprises intron 2 and intron 6.
17. The transgenic mouse of clause 1, any other suitable clause, or any combination of suitable clauses, wherein the hmTert gene comprises SEQ ID NO:2, and wherein SEQ ID NO:2 comprises a 5′ intergenic region (5′IR).
18. The transgenic mouse of clause 1, any other suitable clause, or any combination of suitable clauses, wherein the hmTert gene comprises SEQ ID NO:2, and wherein SEQ ID NO:2 comprises a 5′IR and intron 2.
19. The transgenic mouse of clause 1, any other suitable clause, or any combination of suitable clauses, wherein the hmTert gene comprises SEQ ID NO:2, and wherein SEQ ID NO:2 comprises a 5′IR and intron 6.
20. The transgenic mouse of clause 1, any other suitable clause, or any combination of suitable clauses, wherein the hmTert gene comprises SEQ ID NO:2, and wherein SEQ ID NO:2 comprises a 5′IR and intron 2 and intron 6.
21. The transgenic mouse of clause 1, any other suitable clause, or any combination of suitable clauses, wherein the hmTert gene comprises SEQ ID NO:3.
22. The transgenic mouse of clause 1, any other suitable clause, or any combination of suitable clauses, wherein the hmTert gene consists essentially of SEQ ID NO:3.
23. The transgenic mouse of clause 1, any other suitable clause, or any combination of suitable clauses, wherein the hmTert gene consists of SEQ ID NO:3.
24. The transgenic mouse of clause 1, any other suitable clause, or any combination of suitable clauses, wherein the hmTert gene has at least 80% sequence identity to SEQ ID NO:3.
25. The transgenic mouse of clause 1, any other suitable clause, or any combination of suitable clauses, wherein the hmTert gene has at least 85% sequence identity to SEQ ID NO:3.
26. The transgenic mouse of clause 1, any other suitable clause, or any combination of suitable clauses, wherein the hmTert gene has at least 90% sequence identity to SEQ ID NO:3.
27. The transgenic mouse of clause 1, any other suitable clause, or any combination of suitable clauses, wherein the hmTert gene has at least 95% sequence identity to SEQ ID NO:3.
28. The transgenic mouse of clause 1, any other suitable clause, or any combination of suitable clauses, wherein the hmTert gene has at least 96% sequence identity to SEQ ID NO:3.
29. The transgenic mouse of clause 1, any other suitable clause, or any combination of suitable clauses, wherein the hmTert gene has at least 97% sequence identity to SEQ ID NO:3.
30. The transgenic mouse of clause 1, any other suitable clause, or any combination of suitable clauses, wherein the hmTert gene has at least 98% sequence identity to SEQ ID NO:3.
31. The transgenic mouse of clause 1, any other suitable clause, or any combination of suitable clauses, wherein the hmTert gene has at least 99% sequence identity to SEQ ID NO:3.
32. The transgenic mouse of clause 1, any other suitable clause, or any combination of suitable clauses, wherein the hmTert gene comprises SEQ ID NO:3, and wherein SEQ ID NO:3 comprises intron 2.
33. The transgenic mouse of clause 1, any other suitable clause, or any combination of suitable clauses, wherein the hmTert gene comprises SEQ ID NO:3, and wherein SEQ ID NO:3 comprises intron 6.
34. The transgenic mouse of clause 1, any other suitable clause, or any combination of suitable clauses, wherein the hmTert gene comprises SEQ ID NO:3, and wherein SEQ ID NO:3 comprises intron 2 and intron 6.
35. The transgenic mouse of clause 1, any other suitable clause, or any combination of suitable clauses, wherein the hmTert gene comprises SEQ ID NO:3, and wherein SEQ ID NO:3 comprises a 5′ intergenic region (5′IR).
36. The transgenic mouse of clause 1, any other suitable clause, or any combination of suitable clauses, wherein the hmTert gene comprises SEQ ID NO:3, and wherein SEQ ID NO:3 comprises a 5′IR and intron 2.
37. The transgenic mouse of clause 1, any other suitable clause, or any combination of suitable clauses, wherein the hmTert gene comprises SEQ ID NO:3, and wherein SEQ ID NO:3 comprises a 5′IR and intron 6.
38. The transgenic mouse of clause 1, any other suitable clause, or any combination of suitable clauses, wherein the hmTert gene comprises SEQ ID NO:3, and wherein SEQ ID NO:3 comprises a 5′IR and intron 2 and intron 6.
39. The transgenic mouse of clause 1, any other suitable clause, or any combination of suitable clauses, wherein the background murine germline is from a species of Mus genus.
40. The transgenic mouse of clause 1, any other suitable clause, or any combination of suitable clauses, wherein the background murine germline is a strain of a Mus musculus line.
41. The transgenic mouse of clause 1, any other suitable clause, or any combination of suitable clauses, wherein the background murine germline is a substrain of C57BL/6 line.
42. The transgenic mouse of clause 1, any other suitable clause, or any combination of suitable clauses, wherein the background murine germline is a substrain of J:NU.
43. The transgenic mouse of clause 1, any other suitable clause, or any combination of suitable clauses, wherein the background murine germline is a substrain of NU/J.
44. The transgenic mouse of clause 1, any other suitable clause, or any combination of suitable clauses, wherein the background murine germline is a substrain of 129S1/SvImJ.
45. The transgenic mouse of clause 1, any other suitable clause, or any combination of suitable clauses, wherein the background murine germline is a substrain of 129X1/SvJ.
46. The transgenic mouse of clause 1, any other suitable clause, or any combination of suitable clauses, wherein the background murine germline is a substrain of A/J.
47. The transgenic mouse of clause 1, any other suitable clause, or any combination of suitable clauses, wherein the background murine germline is a substrain of AKR/J.
48. The transgenic mouse of clause 1, any other suitable clause, or any combination of suitable clauses, wherein the background murine germline is a substrain of BALB/cByJ.
49. The transgenic mouse of clause 1, any other suitable clause, or any combination of suitable clauses, wherein the background murine germline is a substrain of BALB/cJ.
50. The transgenic mouse of clause 1, any other suitable clause, or any combination of suitable clauses, wherein the background murine germline is a substrain of C3H/HeJ.
51. The transgenic mouse of clause 1, any other suitable clause, or any combination of suitable clauses, wherein the background murine germline is a substrain of C57BL/6J.
52. The transgenic mouse of clause 1, any other suitable clause, or any combination of suitable clauses, wherein the background murine germline is a substrain of C57BL/6NJ.
53. The transgenic mouse of clause 1, any other suitable clause, or any combination of suitable clauses, wherein the background murine germline is a substrain of C57BL/10J.
54. The transgenic mouse of clause 1, any other suitable clause, or any combination of suitable clauses, wherein the background murine germline is a substrain of CBA/J.
55. The transgenic mouse of clause 1, any other suitable clause, or any combination of suitable clauses, wherein the background murine germline is a substrain of CBA/CaJ.
56. The transgenic mouse of clause 1, any other suitable clause, or any combination of suitable clauses, wherein the background murine germline is a substrain of DBA/1J.
57. The transgenic mouse of clause 1, any other suitable clause, or any combination of suitable clauses, wherein the background murine germline is a substrain of DBA/2J.
58. The transgenic mouse of clause 1, any other suitable clause, or any combination of suitable clauses, wherein the background murine germline is a substrain of FVB/NJ.
59. The transgenic mouse of clause 1, any other suitable clause, or any combination of suitable clauses, wherein the background murine germline is a substrain of C57BL/6J-Apc^{Min}/J.
60. The transgenic mouse of clause 1, any other suitable clause, or any combination of suitable clauses, wherein the transgenic mouse comprises expression maintenance of the hmTert gene.
61. The transgenic mouse of clause 1, any other suitable clause, or any combination of suitable clauses, wherein the transgenic mouse comprises humanized telomere homeostasis.
62. The transgenic mouse of clause 61, any other suitable clause, or any combination of suitable clauses, wherein the telomere homeostasis is maintained upon interbreeding for one or more generations.
63. The transgenic mouse of clause 1, any other suitable clause, or any combination of suitable clauses, wherein the mouse comprises one or more telomeres, and wherein the telomeres comprise an average telomere length from five to twenty (5-20) kilobase (kb).
64. The transgenic mouse of clause 63, any other suitable clause, or any combination of suitable clauses, wherein the telomeres comprise an average telomere length from 5-20 kb.
65. The transgenic mouse of clause 63, any other suitable clause, or any combination of suitable clauses, wherein the telomeres comprise an average telomere length from 5-15 kb.
66. The transgenic mouse of clause 63, any other suitable clause, or any combination of suitable clauses, wherein the telomeres comprise an average telomere length from 5-10 kb.
67. The transgenic mouse of clause 63, any other suitable clause, or any combination of suitable clauses, wherein the telomeres comprise an average telomere length from 6-20 kb.
68. The transgenic mouse of clause 63, any other suitable clause, or any combination of suitable clauses, wherein the telomeres comprise an average telomere length from 6-18 kb.
69. The transgenic mouse of clause 63, any other suitable clause, or any combination of suitable clauses, wherein the telomeres comprise an average telomere length from 6-16 kb.
70. The transgenic mouse of clause 63, any other suitable clause, or any combination of suitable clauses, wherein the telomeres comprise an average telomere length from 6-14 kb.
71. The transgenic mouse of clause 63, any other suitable clause, or any combination of suitable clauses, wherein the telomeres comprise an average telomere length from 6-12 kb.
72. The transgenic mouse of clause 63, any other suitable clause, or any combination of suitable clauses, wherein the telomeres comprise an average telomere length from 6-10 kb.
73. The transgenic mouse of clause 63, any other suitable clause, or any combination of suitable clauses, wherein the telomeres comprise an average telomere length from 6-8 kb.
74. The transgenic mouse of clause 63, any other suitable clause, or any combination of suitable clauses, wherein the telomeres comprise an average telomere length from 8-20 kb.
75. The transgenic mouse of clause 63, any other suitable clause, or any combination of suitable clauses, wherein the telomeres comprise an average telomere length from 8-18 kb.
76. wherein the telomeres comprise an average telomere length from 8-16 kb.
77. The transgenic mouse of clause 63, any other suitable clause, or any combination of suitable clauses, wherein the telomeres comprise an average telomere length from 8-14 kb.
78. The transgenic mouse of clause 63, any other suitable clause, or any combination of suitable clauses, wherein the telomeres comprise an average telomere length from 8-12 kb.
79. The transgenic mouse of clause 63, any other suitable clause, or any combination of suitable clauses, wherein the telomeres comprise an average telomere length from 8-10 kb.
80. The transgenic mouse of clause 63, any other suitable clause, or any combination of suitable clauses, wherein the telomeres comprise an average telomere length from 10-20 kb.
81. The transgenic mouse of clause 63, any other suitable clause, or any combination of suitable clauses, wherein the telomeres comprise an average telomere length from 10-18 kb.
82. The transgenic mouse of clause 63, any other suitable clause, or any combination of suitable clauses, wherein the telomeres comprise an average telomere length from 10-16 kb.
83. The transgenic mouse of clause 63, any other suitable clause, or any combination of suitable clauses, wherein the telomeres comprise an average telomere length from 10-14 kb.
84. The transgenic mouse of clause 63, any other suitable clause, or any combination of suitable clauses, wherein the telomeres comprise an average telomere length from 10-12 kb.
85. The transgenic mouse of clause 63, any other suitable clause, or any combination of suitable clauses, wherein the telomeres comprise an average telomere length from 12-20 kb.
86. The transgenic mouse of clause 63, any other suitable clause, or any combination of suitable clauses, wherein the telomeres comprise an average telomere length from 12-18 kb.
87. The transgenic mouse of clause 63, any other suitable clause, or any combination of suitable clauses, wherein the telomeres comprise an average telomere length from 12-16 kb.
88. The transgenic mouse of clause 63, any other suitable clause, or any combination of suitable clauses, wherein the telomeres comprise an average telomere length from 12-14 kb.
89. The transgenic mouse of clause 63, any other suitable clause, or any combination of suitable clauses, wherein the telomeres comprise an average telomere length from 14-20 kb.
90. The transgenic mouse of clause 63, any other suitable clause, or any combination of suitable clauses, wherein the telomeres comprise an average telomere length from 14-18 kb.
91. The transgenic mouse of clause 63, any other suitable clause, or any combination of suitable clauses, wherein the telomeres comprise an average telomere length from 14-16 kb.
92. The transgenic mouse of clause 63, any other suitable clause, or any combination of suitable clauses, wherein the telomeres comprise an average telomere length from 16-20 kb.
93. The transgenic mouse of clause 63, any other suitable clause, or any combination of suitable clauses, wherein the telomeres comprise an average telomere length from 16-18 kb.
94. The transgenic mouse of clause 63, any other suitable clause, or any combination of suitable clauses, wherein the telomeres comprise an average telomere length less than 20 kb.
95. The transgenic mouse of clause 63, any other suitable clause, or any combination of suitable clauses, wherein the telomeres comprise an average telomere length less than 15 kb.
96. The transgenic mouse of clause 63, any other suitable clause, or any combination of suitable clauses, wherein the telomeres comprise an average telomere length less than 10 kb.
97. A method for evaluating a treatment, the method comprising steps of i) administering the treatment to a transgenic mouse and ii) analyzing a response to the treatment in the transgenic mouse, wherein the response provides evaluation of the treatment.
98. The method of clause 97, any other suitable clause, or any combination of suitable clauses, wherein the treatment is a drug.
99. The method of clause 98, any other suitable clause, or any combination of suitable clauses, wherein the drug is used to treat aplastic anemia.
100. The method of clause 98, any other suitable clause, or any combination of suitable clauses, wherein the drug is used to treat idiopathic aplastic anemia.
101. The method of clause 98, any other suitable clause, or any combination of suitable clauses, wherein the drug is used to treat pulmonary fibrosis.
102. The method of clause 98, any other suitable clause, or any combination of suitable clauses, wherein the drug is used to treat familial idiopathic pulmonary fibrosis.
103. The method of clause 98, any other suitable clause, or any combination of suitable clauses, wherein the drug is used to treat dyskeratosis cogenita.
104. The method of clause 98, any other suitable clause, or any combination of suitable clauses, wherein the drug is used to treat hepatic disease.
105. The method of clause 98, any other suitable clause, or any combination of suitable clauses, wherein the drug is used to treat cirrhosis with inflammation
106. The method of clause 98, any other suitable clause, or any combination of suitable clauses, wherein the drug is used to treat nodular regenerative hyperplasia.
107. The method of clause 98, any other suitable clause, or any combination of suitable clauses, wherein the drug is used to treat cancer.
108. The method of clause 107, any other suitable clause, or any combination of suitable clauses, wherein the cancer is carcinoma with nonreciprocal translocations.
109. The method of clause 107, any other suitable clause, or any combination of suitable clauses, wherein the cancer is colorectal cancer.
110. The method of clause 107, any other suitable clause, or any combination of suitable clauses, wherein the cancer is esophageal cancer.
111. The method of clause 107, any other suitable clause, or any combination of suitable clauses, wherein the cancer is head and neck squamous cell carcinoma.
112. The method of clause 107, any other suitable clause, or any combination of suitable clauses, wherein the cancer is skin cancer.
113. The method of clause 107, any other suitable clause, or any combination of suitable clauses, wherein the cancer is anorectal cancer.
114. The method of clause 107, any other suitable clause, or any combination of suitable clauses, wherein the cancer is acute myeloid leukemia.
115. The method of clause 107, any other suitable clause, or any combination of suitable clauses, wherein the cancer is leukemic transformation in myelodysplasia.
116. The method of clause 107, any other suitable clause, or any combination of suitable clauses, wherein the cancer is secondary myelodysplasia.
117. The method of clause 107, any other suitable clause, or any combination of suitable clauses, wherein the cancer is secondary leukemia.
118. The method of clause 107, any other suitable clause, or any combination of suitable clauses, wherein the cancer is basal cell cancer of the skin.
119. The method of clause 107, any other suitable clause, or any combination of suitable clauses, wherein the cancer is cancer of the lung.
120. The method of clause 107, any other suitable clause, or any combination of suitable clauses, wherein the cancer is cancer of the bladder.
121. The method of clause 107, any other suitable clause, or any combination of suitable clauses, wherein the cancer is cancer of the prostate.
122. The method of clause 107, any other suitable clause, or any combination of suitable clauses, wherein the cancer is cancer of the cervix.
123. The method of clause 107, any other suitable clause, or any combination of suitable clauses, wherein the cancer is glioblastoma.
124. The method of clause 107, any other suitable clause, or any combination of suitable clauses, wherein the cancer is renal cell carcinoma.
125. The method of clause 107, any other suitable clause, or any combination of suitable clauses, wherein the cancer is Barrett's esophagus hepatocellular carcinoma.
126. The method of clause 98, any other suitable clause, or any combination of suitable clauses, wherein the drug is used to treat inflammatory bowel disease.
127. The method of clause 98, any other suitable clause, or any combination of suitable clauses, wherein the drug is used to treat chronic hepatitis B infection.
128. The method of clause 98, any other suitable clause, or any combination of suitable clauses, wherein the drug is used to treat ulcerative colitis.
129. The method of clause 98, any other suitable clause, or any combination of suitable clauses, wherein the drug is used to treat chronic graft-versus-host disease.
130. The method of clause 98, any other suitable clause, or any combination of suitable clauses, wherein the drug is used to treat cardiovascular disease.
131. The method of clause 97, any other suitable clause, or any combination of suitable clauses, wherein the treatment is radiation.
132. The method of clause 97, any other suitable clause, or any combination of suitable clauses, wherein the treatment is surgery.
133. The method of clause 97, any other suitable clause, or any combination of suitable clauses, wherein the treatment is a transplant.
134. The method of clause 97, any other suitable clause, or any combination of suitable clauses, wherein the treatment is immunotherapy.
135. The method of clause 97, any other suitable clause, or any combination of suitable clauses, wherein the treatment is gene therapy.
136. The method of clause 97, any other suitable clause, or any combination of suitable clauses, wherein the treatment is biological engineering.
137. The method of clause 97, any other suitable clause, or any combination of suitable clauses, wherein the treatment is a disease-causing agent.
138. The method of clause 137, any other suitable clause, or any combination of suitable clauses, wherein the disease-causing agent is a pathogen.
139. The method of clause 137, any other suitable clause, or any combination of suitable clauses, wherein the disease-causing agent is a carcinogen.
140. The method of clause 137, any other suitable clause, or any combination of suitable clauses, wherein the disease-causing agent is a cancer cell.
141. The method of clause 97, any other suitable clause, or any combination of suitable clauses, wherein the administering comprises an injection.
142. The method of clause 97, any other suitable clause, or any combination of suitable clauses, wherein the administering comprises oral administration.
143. The method of clause 97, any other suitable clause, or any combination of suitable clauses, wherein the administering comprises topical application.
144. The method of clause 97, any other suitable clause, or any combination of suitable clauses, wherein the administering comprises inhalation.
145. The method of clause 97, any other suitable clause, or any combination of suitable clauses, wherein the administering comprises radiation.
146. The method of clause 97, any other suitable clause, or any combination of suitable clauses, wherein the administering comprises transplantation.
147. The method of clause 97, any other suitable clause, or any combination of suitable clauses, wherein the response is an immune system response.
148. The method of clause 97, any other suitable clause, or any combination of suitable clauses, wherein the response is tumorigenesis.
149. The method of clause 97, any other suitable clause, or any combination of suitable clauses, wherein the response is senescence.
150. The method of clause 97, any other suitable clause, or any combination of suitable clauses, wherein the response is cellular aging.
151. The method of clause 97, any other suitable clause, or any combination of suitable clauses, wherein the response is a compromised innate immune response.
152. The method of clause 97, any other suitable clause, or any combination of suitable clauses, wherein the response is a compromised adaptive immune response.
153. The method of clause 97, any other suitable clause, or any combination of suitable clauses, wherein the response is apoptosis.
154. The method of clause 97, any other suitable clause, or any combination of suitable clauses, wherein the response is oncogenic transformation.
155. The method of clause 97, any other suitable clause, or any combination of suitable clauses, wherein the response is side effects.
156. The method of clause 97, any other suitable clause, or any combination of suitable clauses, wherein the response is presence of disease.
157. The method of clause 97, any other suitable clause, or any combination of suitable clauses, wherein the response is telomere length.
158. The method of clause 97, any other suitable clause, or any combination of suitable clauses, wherein the response is telomerase activity.
159. The method of clause 97, any other suitable clause, or any combination of suitable clauses, wherein the response is cellular metabolic activity.
160. The method of clause 97, any other suitable clause, or any combination of suitable clauses, wherein the response is gene expression.
161. The method of clause 97, any other suitable clause, or any combination of suitable clauses, wherein the response is RNA expression.
162. The method of clause 97, any other suitable clause, or any combination of suitable clauses, wherein the response is protein expression.
163. The method of clause 97, any other suitable clause, or any combination of suitable clauses, wherein the response is to cancer.
164. The method of clause 163, any other suitable clause, or any combination of suitable clauses, wherein the cancer is melanoma.
165. The method of clause 163, any other suitable clause, or any combination of suitable clauses, wherein the cancer is adrenal gland cancer.
166. The method of clause 163, any other suitable clause, or any combination of suitable clauses, wherein the cancer is bladder cancer.
167. The method of clause 163, any other suitable clause, or any combination of suitable clauses, wherein the cancer is bone cancer.
168. The method of clause 163, any other suitable clause, or any combination of suitable clauses, wherein the cancer is blood cancer.
169. The method of clause 163, any other suitable clause, or any combination of suitable clauses, wherein the cancer is brain cancer.
170. The method of clause 163, any other suitable clause, or any combination of suitable clauses, wherein the cancer is breast cancer.
171. The method of clause 163, any other suitable clause, or any combination of suitable clauses, wherein the cancer is cervical cancer.
172. The method of clause 163, any other suitable clause, or any combination of suitable clauses, wherein the cancer is colon cancer.
173. The method of clause 163, any other suitable clause, or any combination of suitable clauses, wherein the cancer is endometrium cancer.
174. The method of clause 163, any other suitable clause, or any combination of suitable clauses, wherein the cancer is esophageal cancer.
175. The method of clause 163, any other suitable clause, or any combination of suitable clauses, wherein the cancer is eye cancer.
176. The method of clause 163, any other suitable clause, or any combination of suitable clauses, wherein the cancer is head and neck cancer.
177. The method of clause 163, any other suitable clause, or any combination of suitable clauses, wherein the cancer is intestinal cancer.
178. The method of clause 163, any other suitable clause, or any combination of suitable clauses, wherein the cancer is kidney cancer.
179. The method of clause 163, any other suitable clause, or any combination of suitable clauses, wherein the cancer is liver cancer.
180. The method of clause 163, any other suitable clause, or any combination of suitable clauses, wherein the cancer is lung cancer.
181. The method of clause 163, any other suitable clause, or any combination of suitable clauses, wherein the cancer is mouth cancer.
182. The method of clause 163, any other suitable clause, or any combination of suitable clauses, wherein the cancer is muscle cancer.
183. The method of clause 163, any other suitable clause, or any combination of suitable clauses, wherein the cancer is nose cancer.
184. The method of clause 163, any other suitable clause, or any combination of suitable clauses, wherein the cancer is ovarian cancer.
185. The method of clause 163, any other suitable clause, or any combination of suitable clauses, wherein the cancer is pancreatic cancer.
186. The method of clause 163, any other suitable clause, or any combination of suitable clauses, wherein the cancer is pituitary cancer.
187. The method of clause 163, any other suitable clause, or any combination of suitable clauses, wherein the cancer is prostate cancer.
188. The method of clause 163, any other suitable clause, or any combination of suitable clauses, wherein the cancer is skin cancer.
189. The method of clause 163, any other suitable clause, or any combination of suitable clauses, wherein the cancer is stomach cancer.
190. The method of clause 163, any other suitable clause, or any combination of suitable clauses, wherein the cancer is thymus cancer.
191. The method of clause 163, any other suitable clause, or any combination of suitable clauses, wherein the cancer is thyroid cancer.
192. The method of clause 163, any other suitable clause, or any combination of suitable clauses, wherein the cancer is testicular cancer.
193. The method of clause 163, any other suitable clause, or any combination of suitable clauses, wherein the cancer is an immune system cancer.
194. The method of clause 163, any other suitable clause, or any combination of suitable clauses, wherein the cancer is a nervous system cancer.
195. The method of clause 163, any other suitable clause, or any combination of suitable clauses, wherein the cancer is a neuroendocrine system cancer.
196. The method of clause 163, any other suitable clause, or any combination of suitable clauses, wherein the cancer is lymphoproliferative disease.
197. The method of clause 163, any other suitable clause, or any combination of suitable clauses, wherein the cancer is a hematologic malignancy.
198. The method of clause 163, any other suitable clause, or any combination of suitable clauses, wherein the cancer is a myelodysplastic syndrome.
199. The method of clause 163, any other suitable clause, or any combination of suitable clauses, wherein the cancer is acute myeloid leukemia.
200. The method of clause 97, any other suitable clause, or any combination of suitable clauses, wherein the response is to a rejuvenation or lifespan extension.
201. The method of clause 200, any other suitable clause, or any combination of suitable clauses, wherein the rejuvenation or lifespan extension is a telomerase-mediated rejuvenation.
202. The method of clause 200, any other suitable clause, or any combination of suitable clauses, wherein the rejuvenation or lifespan extension is epigenetic reprogramming.
203. The method of clause 200, any other suitable clause, or any combination of suitable clauses, wherein the rejuvenation or lifespan extension is to a senolytic therapy 204. The method of clause 200, any other suitable clause, or any combination of suitable clauses, wherein the rejuvenation or lifespan extension is to an immunotherapy.
205. The method of clause 97, any other suitable clause, or any combination of suitable clauses, wherein the response is skin appearance.
206. The method of clause 97, any other suitable clause, or any combination of suitable clauses, wherein the response is hair loss/growth.
207. The method of clause 97, any other suitable clause, or any combination of suitable clauses, wherein the response is nail aging/rejuvenation.
208. The method of clause 97, any other suitable clause, or any combination of suitable clauses, wherein the response is to skincare products.
209. The method of clause 97, any other suitable clause, or any combination of suitable clauses, wherein the response is to cosmetics
210. The method of clause 97, any other suitable clause, or any combination of suitable clauses, wherein the response is to hair graying.
211. The method of clause 97, any other suitable clause, or any combination of suitable clauses, wherein the response is to alopecia.
212. The method of clause 97, any other suitable clause, or any combination of suitable clauses, wherein the response is to hair loss treatment.
213. The method of clause 97, any other suitable clause, or any combination of suitable clauses, wherein the response is to hyperpigmentation.
214. The method of clause 97, any other suitable clause, or any combination of suitable clauses, wherein the response is to hypopigmentation.
215. The method of clause 97, any other suitable clause, or any combination of suitable clauses, wherein the response is to abnormal skin pigmentation.
216. The method of clause 97, any other suitable clause, or any combination of suitable clauses, wherein the response is to nail dystrophy.
217. The method of clause 97, any other suitable clause, or any combination of suitable clauses, wherein the response is to adermatoglyphia.
218. The method of clause 97, any other suitable clause, or any combination of suitable clauses, wherein the response is to shortened telomeres.
219. The method of clause 218, any other suitable clause, or any combination of suitable clauses, wherein the shortened telomeres result from Coats Plus syndrome.
220. The method of clause 218, any other suitable clause, or any combination of suitable clauses, wherein the shortened telomeres result from Fanconi anemia.
221. The method of clause 218, any other suitable clause, or any combination of suitable clauses, wherein the shortened telomeres result from Revesz syndrome.
222. The method of clause 218, any other suitable clause, or any combination of suitable clauses, wherein the shortened telomeres result from Hoyeraal-Hreidarsson syndrome.
223. The method of clause 97, any other suitable clause, or any combination of suitable clauses, wherein the response is to telomere extension.
224. The method of clause 223, any other suitable clause, or any combination of suitable clauses, wherein the telomere extension results from telomerase activation.
225. The method of clause 223, any other suitable clause, or any combination of suitable clauses, wherein the telomere extension results from epigenetic reprogramming.
226. The method of clause 97, any other suitable clause, or any combination of suitable clauses, wherein the response is to stem cell proliferation and maintenance.
227. The method of clause 226, any other suitable clause, or any combination of suitable clauses, wherein the stem cell proliferation and maintenance comprises telomere-mediated stem cell failures manifested in lesions of skin, hematopoietic, gastrointestinal, and reproductive systems.
228. The method of clause 97, any other suitable clause, or any combination of suitable clauses, wherein the response is to an immunologic system reaction.
229. The method of clause 97, any other suitable clause, or any combination of suitable clauses, wherein the response is to a pharmaceutical event.
230. The method of clause 97, any other suitable clause, or any combination of suitable clauses, wherein the response is to an environmental exposure.
231. The method of clause 97, any other suitable clause, or any combination of suitable clauses, wherein the response is to a human lifestyle event.
232. The method of clause 97, any other suitable clause, or any combination of suitable clauses, wherein the response is to a disease.
233. The method of clause 232, any other suitable clause, or any combination of suitable clauses, wherein the disease is an aging or age-related disease.
234. The method of clause 233, any other suitable clause, or any combination of suitable clauses, wherein the aging or age-related disease is senescence/aging.
235. The method of clause 233, any other suitable clause, or any combination of suitable clauses, wherein the aging or age-related disease is longevity.
236. The method of clause 233, any other suitable clause, or any combination of suitable clauses, wherein the aging or age-related disease is Dyskeratosis congenita (DC).
237. The method of clause 233, any other suitable clause, or any combination of suitable clauses, wherein the aging or age-related disease is Werner syndrome.
238. The method of clause 233, any other suitable clause, or any combination of suitable clauses, wherein the aging or age-related disease is Bloom syndrome.
239. The method of clause 233, any other suitable clause, or any combination of suitable clauses, wherein the aging or age-related disease is Hutchinson-Gilford progeria syndrome.
240. The method of clause 232, any other suitable clause, or any combination of suitable clauses, wherein the disease is a digestive system disease.
241. The method of clause 240, any other suitable clause, or any combination of suitable clauses, wherein the digestive system disease is oral leukoplakia.
242. The method of clause 240, any other suitable clause, or any combination of suitable clauses, wherein the digestive system disease is oesophageal structures and webs.
243. The method of clause 240, any other suitable clause, or any combination of suitable clauses, wherein the digestive system disease is telangiectasias prone to hemorrhage.
244. The method of clause 240, any other suitable clause, or any combination of suitable clauses, wherein the digestive system disease is villus atrophy.
245. The method of clause 240, any other suitable clause, or any combination of suitable clauses, wherein the digestive system disease is enteropathy.
246. The method of clause 232, any other suitable clause, or any combination of suitable clauses, wherein the disease is a lung disease.
247. The method of clause 246, any other suitable clause, or any combination of suitable clauses, wherein the lung disease is pulmonary fibrosis (PF).
248. The method of clause 246, any other suitable clause, or any combination of suitable clauses, wherein the lung disease is chronic obstructive pulmonary disease (COPD).
249. The method of clause 246, any other suitable clause, or any combination of suitable clauses, wherein the lung disease is emphysema.
250. The method of clause 246, any other suitable clause, or any combination of suitable clauses, wherein the lung disease is non-cystic fibrosis bronchiectasis.
251. The method of clause 232, any other suitable clause, or any combination of suitable clauses, wherein the disease is a neurodegenerative disease.
252. The method of clause 251, any other suitable clause, or any combination of suitable clauses, wherein the neurodegenerative disease is dementia.
253. The method of clause 251, any other suitable clause, or any combination of suitable clauses, wherein the neurodegenerative disease is Alzheimer's disease (AD).
254. The method of clause 251, any other suitable clause, or any combination of suitable clauses, wherein the neurodegenerative disease is Parkinson's disease (PD).
255. The method of clause 251, any other suitable clause, or any combination of suitable clauses, wherein the neurodegenerative disease is Huntington's disease (HD).
256. The method of clause 232, any other suitable clause, or any combination of suitable clauses, wherein the disease is an eye disease.
257. The method of clause 256, any other suitable clause, or any combination of suitable clauses, wherein the eye disease is age-related macular degeneration.
258. The method of clause 232, any other suitable clause, or any combination of suitable clauses, wherein the disease is a bone marrow disease.
259. The method of clause 258, any other suitable clause, or any combination of suitable clauses, wherein the bone marrow disease is aplastic anemia.
260. The method of clause 258, any other suitable clause, or any combination of suitable clauses, wherein the bone marrow disease is bone marrow failure.
261. The method of clause 258, any other suitable clause, or any combination of suitable clauses, wherein the bone marrow disease is myelodysplastic syndrome (MDS).
262. The method of clause 232, any other suitable clause, or any combination of suitable clauses, wherein the disease is an immune system reaction.
263. The method of clause 262, any other suitable clause, or any combination of suitable clauses, wherein the immune system reaction is an innate immune reaction.
264. The method of clause 262, any other suitable clause, or any combination of suitable clauses, wherein the immune system reaction is an adaptive immune reaction.
265. The method of clause 262, any other suitable clause, or any combination of suitable clauses, wherein the immune system reaction is immune cell senescence and exhaustion.
266. The method of clause 262, any other suitable clause, or any combination of suitable clauses, wherein the immune system reaction is a B cell lymphopenia/immunodeficiency.
267. The method of clause 262, any other suitable clause, or any combination of suitable clauses, wherein the immune system reaction is a T cell lymphopenia/immunodeficiency.
268. The method of clause 262, any other suitable clause, or any combination of suitable clauses, wherein the immune system reaction is a natural killer (NK) cell lymphopenia/immunodeficiency.
269. The method of clause 232, any other suitable clause, or any combination of suitable clauses, wherein the disease is an infectious disease.
270. The method of clause 269, any other suitable clause, or any combination of suitable clauses, wherein the infectious disease is caused by HIV.
271. The method of clause 269, any other suitable clause, or any combination of suitable clauses, wherein the infectious disease is caused by SARS-CoV-2.
272. The method of clause 269, any other suitable clause, or any combination of suitable clauses, wherein the infectious disease is caused by a hepatitis virus.
273. The method of clause 232, any other suitable clause, or any combination of suitable clauses, wherein the disease is an inflammatory disease.
274. The method of clause 273, any other suitable clause, or any combination of suitable clauses, wherein the inflammatory disease is inflammatory bowel disease (IBD).
275. The method of clause 273, any other suitable clause, or any combination of suitable clauses, wherein the inflammatory disease is Crohn's disease (CD).
276. The method of clause 273, any other suitable clause, or any combination of suitable clauses, wherein the inflammatory disease is ulcerative colitis (UC).
277. The method of clause 273, any other suitable clause, or any combination of suitable clauses, wherein the inflammatory disease is rheumatoid arthritis.
278. The method of clause 273, any other suitable clause, or any combination of suitable clauses, wherein the inflammatory disease is atherosclerosis.
279. The method of clause 273, any other suitable clause, or any combination of suitable clauses, wherein the inflammatory disease is a chronic renal disease.
280. The method of clause 232, any other suitable clause, or any combination of suitable clauses, wherein the disease is a metabolic disease.
281. The method of clause 280, any other suitable clause, or any combination of suitable clauses, wherein the metabolic disease is chronic inflammation.
282. The method of clause 280, any other suitable clause, or any combination of suitable clauses, wherein the metabolic disease is obesity.
283. The method of clause 280, any other suitable clause, or any combination of suitable clauses, wherein the metabolic disease is dyslipidaemia.
284. The method of clause 280, any other suitable clause, or any combination of suitable clauses, wherein the metabolic disease is hypertension.
285. The method of clause 280, any other suitable clause, or any combination of suitable clauses, wherein the metabolic disease is insulin resistance.
286. The method of clause 280, any other suitable clause, or any combination of suitable clauses, wherein the metabolic disease is diabetes.
287. The method of clause 232, any other suitable clause, or any combination of suitable clauses, wherein the disease is a hepatic disease.
288. The method of clause 287, any other suitable clause, or any combination of suitable clauses, wherein the hepatic disease is cirrhosis.
289. The method of clause 287, any other suitable clause, or any combination of suitable clauses, wherein the hepatic disease is non-alcoholic fatty liver disease (NAFLD).
290. The method of clause 287, any other suitable clause, or any combination of suitable clauses, wherein the hepatic disease is non-alcoholic steatohepatitis (NASH).
291. The method of clause 287, any other suitable clause, or any combination of suitable clauses, wherein the hepatic disease is fibrosis.
292. The method of clause 287, any other suitable clause, or any combination of suitable clauses, wherein the hepatic disease is primary biliary cirrhosis.
293. The method of clause 287, any other suitable clause, or any combination of suitable clauses, wherein the hepatic disease is alcoholic liver disease.
294. The method of clause 287, any other suitable clause, or any combination of suitable clauses, wherein the hepatic disease is nodular regenerative hyperplasia hepatopulmonary syndrome.
295. The method of clause 287, any other suitable clause, or any combination of suitable clauses, wherein the hepatic disease is chronic viral hepatitis.
296. The method of clause 232, any other suitable clause, or any combination of suitable clauses, wherein the disease is a cardiovascular disease.
297. The method of clause 232, any other suitable clause, or any combination of suitable clauses, wherein the cardiovascular disease is cardiomyocyte hypertrophy.
298. The method of clause 232, any other suitable clause, or any combination of suitable clauses, wherein the cardiovascular disease is fibrosis.
299. The method of clause 232, any other suitable clause, or any combination of suitable clauses, wherein the cardiovascular disease is cardiac ischaemia-reperfusion injury (IRI).
300. The method of clause 232, any other suitable clause, or any combination of suitable clauses, wherein the cardiovascular disease is hypertrophic or dilated cardiomyopathy.
301. The method of clause 232, any other suitable clause, or any combination of suitable clauses, wherein the cardiovascular disease is myocardial infarction.
302. The method of clause 232, any other suitable clause, or any combination of suitable clauses, wherein the cardiovascular disease is atherosclerosis.
303. The method of clause 232, any other suitable clause, or any combination of suitable clauses, wherein the disease is a musculoskeletal disease.
304. The method of clause 303, any other suitable clause, or any combination of suitable clauses, wherein the musculoskeletal disease is osteopenia.
305. The method of clause 303, any other suitable clause, or any combination of suitable clauses, wherein the musculoskeletal disease is osteoarthritis.
306. The method of clause 303, any other suitable clause, or any combination of suitable clauses, wherein the musculoskeletal disease is rheumatoid arthritis.
307. The method of clause 303, any other suitable clause, or any combination of suitable clauses, wherein the musculoskeletal disease is osteoporosis.
308. The method of clause 303, any other suitable clause, or any combination of suitable clauses, wherein the musculoskeletal disease is skeletal fragility.
309. The method of clause 303, any other suitable clause, or any combination of suitable clauses, wherein the musculoskeletal disease is avascular necrosis.
310. The method of clause 303, any other suitable clause, or any combination of suitable clauses, wherein the musculoskeletal disease is Duchenne muscular dystrophy (DMD).
311. The method of clause 232, any other suitable clause, or any combination of suitable clauses, wherein the disease is a kidney disease.
312. The method of clause 311, any other suitable clause, or any combination of suitable clauses, wherein the kidney disease is an acute kidney injury.
313. The method of clause 311, any other suitable clause, or any combination of suitable clauses, wherein the kidney disease is glomerulonephritis.
314. The method of clause 311, any other suitable clause, or any combination of suitable clauses, wherein the kidney disease is diabetic nephropathy.
315. The method of clause 311, any other suitable clause, or any combination of suitable clauses, wherein the kidney disease is polycystic kidney disease.
316. The method of clause 311, any other suitable clause, or any combination of suitable clauses, wherein the kidney disease is kidney fibrosis.
317. The method of clause 311, any other suitable clause, or any combination of suitable clauses, wherein the kidney disease is chronic kidney disease (CKD).
318. The method of clause 232, any other suitable clause, or any combination of suitable clauses, wherein the disease is a reproductive disease.
319. The method of clause 318, any other suitable clause, or any combination of suitable clauses, wherein the reproductive disease comprises testicular function.
320. The method of clause 318, any other suitable clause, or any combination of suitable clauses, wherein the reproductive disease comprises ovarian function.
321. The method of clause 318, any other suitable clause, or any combination of suitable clauses, wherein the reproductive disease comprises male infertility.
322. The method of clause 318, any other suitable clause, or any combination of suitable clauses, wherein the reproductive disease comprises female infertility.
323. The method of clause 97, any other suitable clause, or any combination of suitable clauses, wherein the response is a susceptibility and resiliency of human health in response to a pharmaceutical intervention.
324. The method of clause 323, any other suitable clause, or any combination of suitable clauses, wherein the pharmaceutical intervention is rapamycin.
325. The method of clause 323, any other suitable clause, or any combination of suitable clauses, wherein the pharmaceutical intervention is 170-oestradial.
326. The method of clause 323, any other suitable clause, or any combination of suitable clauses, wherein the pharmaceutical intervention is senolytic strategies.
327. The method of clause 323, any other suitable clause, or any combination of suitable clauses, wherein the pharmaceutical intervention is drug efficacy.
328. The method of clause 323, any other suitable clause, or any combination of suitable clauses, wherein the pharmaceutical intervention is drug toxicity.
329. The method of clause 323, any other suitable clause, or any combination of suitable clauses, wherein the pharmaceutical intervention is a drug-drug interaction.
330. The method of clause 323, any other suitable clause, or any combination of suitable clauses, wherein the pharmaceutical intervention is a pharmacodynamic study.
331. The method of clause 97, any other suitable clause, or any combination of suitable clauses, wherein the response is susceptibility and resiliency of human health in response to an environmental exposure.
332. The method of clause 331, any other suitable clause, or any combination of suitable clauses, wherein the environmental exposure is air pollution.
333. The method of clause 331, any other suitable clause, or any combination of suitable clauses, wherein the environmental exposure is food poisoning.
334. The method of clause 331, any other suitable clause, or any combination of suitable clauses, wherein the environmental exposure is heavy metal.
335. The method of clause 331, any other suitable clause, or any combination of suitable clauses, wherein the environmental exposure is hazardous working condition.
336. The method of clause 331, any other suitable clause, or any combination of suitable clauses, wherein the environmental exposure is algae.
337. The method of clause 331, any other suitable clause, or any combination of suitable clauses, wherein the environmental exposure is water pollution.
338. The method of clause 331, any other suitable clause, or any combination of suitable clauses, wherein the environmental exposure is a mutagen.
339. The method of clause 331, any other suitable clause, or any combination of suitable clauses, wherein the environmental exposure is radiation.
340. The method of clause 331, any other suitable clause, or any combination of suitable clauses, wherein the environmental exposure is space travel.
341. The method of clause 97, any other suitable clause, or any combination of suitable clauses, wherein the response is susceptibility and resiliency of human health in response to a lifestyle change.
342. The method of clause 341, any other suitable clause, or any combination of suitable clauses, wherein the lifestyle change is dietary restriction.
343. The method of clause 341, any other suitable clause, or any combination of suitable clauses, wherein the lifestyle change is cigarette smoking.
344. The method of clause 341, any other suitable clause, or any combination of suitable clauses, wherein the lifestyle change is exercise.
345. The method of clause 341, any other suitable clause, or any combination of suitable clauses, wherein the lifestyle change is physical work.
346. The method of clause 341, any other suitable clause, or any combination of suitable clauses, wherein the lifestyle change is sleep deprivation.
347. The method of clause 341, any other suitable clause, or any combination of suitable clauses, wherein the lifestyle change is night shift working condition.
348. The method of clause 341, any other suitable clause, or any combination of suitable clauses, wherein the lifestyle change is chronic fatigue.
349. The method of clause 341, any other suitable clause, or any combination of suitable clauses, wherein the lifestyle change is life stress.
350. The method of clause 341, any other suitable clause, or any combination of suitable clauses, wherein the lifestyle change is psychological stress.
351. The method of clause 341, any other suitable clause, or any combination of suitable clauses, wherein the lifestyle change is a psychological disorder.
352. The method of clause 341, any other suitable clause, or any combination of suitable clauses, wherein the lifestyle change is mental health.
353. The method of clause 341, any other suitable clause, or any combination of suitable clauses, wherein the lifestyle change is a psychiatric illness.
354. A method of generating a transgenic mouse, the method comprising steps of:
   inserting an hmTert gene into a background murine germline to form a modified embryonic stem cell; and
   generating a first-generation mouse from the modified embryonic stem cell, wherein the first-generation mouse comprises the hmTert gene.
355. The method of clause 354, any other suitable clause, or any combination of suitable clauses, further comprising breeding one or more generations from the first-generation mouse to generate a progeny mouse.
356. The method of clause 355, any other suitable clause, or any combination of suitable clauses, wherein the breeding one or more generations comprises controlled breeding with one or more of hmTert, mTert, and mTert-knockout strains.
357. The method of clause 355, any other suitable clause, or any combination of suitable clauses, wherein the progeny mouse exhibits average telomere length of five to fifteen (5-15) kilobase.
358. A method of generating a transgenic mouse, the method comprising a step of breeding a first mouse comprising an hmTert gene with a second mouse to generate a heterozygous mouse.
359. The method of clause 358, any other suitable clause, or any combination of suitable clauses, further comprising a step of breeding one or more generations from the heterozygous mouse.

### EXAMPLES

### Example 1

### Exemplary Experimental Procedures

The instant example provides exemplary materials and methods utilized in the examples as described herein.

Generation of Mice with hmTert Alleles.

The engineering of the hmTert gene (FIG. 1A) in mouse ESCs (Tert^{h/+}) was previously reported. The ESCs (G4, 129xC57BL/6) were injected into blastocysts of C57BL/6J albino hosts. The resulting male chimera mice were bred with C57BL/6J female mice. Mice with a germline hmTert allele (F0) were obtained and crossed again with C57BL/61J mice to generate F1 Tert^{+/h} mice with 88% C57BL/6J background. The F1 Tert^{+/h} mice were backcrossed with C57BL/6J mice for three generations before Tert^{h/+} mice were used for mRNA expression analyses. Tert^{+/h} mice were also crossed with Tert-knockout (Tert-KO) mice. All animal experiments were approved by the Institutional Animal Care and Use Committee in accordance with the NIH Guide for Care and Use of Animals.

### Gene Expression and Telomerase Activity.

mRNA expression analyses were performed as previously described and data were normalized to 18S ribosomal RNA. Primer sequences are provided in Table 1, Table 2, and Table 3. Telomerase activities were determined using a modified telomeric repeat amplification protocol (TRAP) assay. Tissues and cell extracts were adjusted to same concentration and 0.5% g samples were used in each reaction. ESCs (Tert^{h/h}, Tert^{+/+}, and Tert^{+/h}) served as positive controls.

TABLE 1
Oligonucleotide Sequences (5′→3′) for the TRAP
assay.

| TRAP assay | | Amplicon |
|---|---|---|
| TS | AATCCGTCGAGCAGAGTT (SEQ ID | |
| | NO: 4) | |
| NT | ATCGCTTCTCGGCCTTTT (SEQ ID | |
| | NO: 5) | |
| TSNT | AATCCGTCGAGCAGAGTTAAAAGG | |
| | CCGAGAAGCGAT (SEQ ID NO: 6) | |
| ACX | GCGCGGCTTACCCTTACCCTTACCC | |
| | TAACC (SEQ ID NO: 7) | |

TABLE 2
Oligonucleotide Sequences (5′→3′) for mouse genotyping.

| Oligos for mouse genotyping | | | Amplicon |
|---|---|---|---|
| hmTert | ATCTCCTGCAGGTTTGCTGT (SEQ | AGAAACGCATCA | 363 |
| | ID NO: 8) | CAGACACG (SEQ | |
| | | ID NO: 9) | |
| mTert | GGTCACCAGGCCCTCGGTGATCTG | TGGAATGTGCTCT | 437 |
| | GATGTGGCATGTC | CTGTGATG (SEQ | |
| | CTTTACAAGGATCACCGGTTCGT | ID NO: 11) | |
| | (SEQ ID NO: 10) | | |
| mTert-KO | CATTGTGGATCTGAGGTGAGTC | CGCCTTCTTGACG | 467 |
| | (SEQ ID NO: 12) | AGTTCTT (SEQ ID | |
| | | NO: 13) | |

TABLE 3
Oligonucleotide Sequences (5′→3′) for Quantitative RT-PCR.

| Quantitative RT-PCR primers | | | Amplicon |
|---|---|---|---|
| hmTert | TGTGGAAGATGAAGGTCG | CCACGTATGTGTCCATCAG | 135 |
| | AA (SEQ ID NO: 14) | C (SEQ ID NO: 15) | |
| mTert | TGAAAGTAGAGGATTGCC | CCACGTATGTGTCCATCAG | 126 |
| | ACTG (SEQ ID NO: 16) | C (SEQ ID NO: 17) | |
| hTERT | GAACATGCGTCGCAAACT | TGCAGCAGGAGGATCTTG | |
| | CTTTGG (SEQ ID NO: 18) | TAGATG (SEQ ID NO: 19) | |
| hmTert&m | GCAGGAACTGATGTGGA | CCACGTATGTGTCCATCAG | For |
| Tert | AGA (SEQ ID NO: 20) | C (SEQ ID NO: 21) | intestine |
| | | | qRT-PCR |
| | | | in |
| | | | h/−, +/−, −/−, |
| | | | +/+ mice |
| p16 | GCTGGGTGGTCTTTGTGT | TTAGCTCTGCTCTTGGGAT | |
| | A (SEQ ID NO: 22) | TG (SEQ ID NO: 23) | |
| p21 | CTGAGCGGCCTGAAGATT | ATCTGCGCTTGGAGTGAT | |
| | (SEQ ID NO: 24) | AG (SEQ ID NO: 25) | |
| Ki67 | CTGTCACTCCAGATCAGA | GGAGGCAGTCTTCATAGT | |
| | ACTC (SEQ ID NO: 26) | CTTC (SEQ ID NO: 27) | |
| PCNA | CGAAGCACCAAATCAAG | CACCCGACGGCATCTTTAT | |
| | AGAAAG (SEQ ID NO: 28) | TA (SEQ ID NO: 29) | |
| TNF-α | CCCCAAAGGGATGAGAA | CACTTGGTGGTTTGCTACG | |
| | GTT (SEQ ID NO: 30) | A (SEQ ID NO: 31) | |
| IL-6 | GTTCTCTGGGAAATCGTG | TTCTGCAAGTGCATCATCG | |
| | GA (SEQ ID NO: 32) | T (SEQ ID NO: 33) | |
| 18s RNA | TAGAGGGACAAGTGGCG | CGCTGAGCCAGTCAGTGT | 104 |
| | TTC (SEQ ID NO: 34) | (SEQ ID NO: 35) | |

### Telomere Length Measurements.

Telomere lengths were measured using two independent methods. TRF analysis was described previously. Genomic DNAs were digested with Hinf I and Rsa I, and subjected to pulsed-field gel electrophoresis using CHEF-DR III Pulsed field Electrophoresis Systems (for telomeres over 20-kb) or regular 0.6% Agarose gel (for telomeres less than 20-kb), followed by Southern blotting using a (TTAGGG)₃-Biotin probe. Probe signal was developed with Chemiluminescent Nucleic Acid Detection Module Kit (Thermo Fisher). Telomere lengths were also measured by Flow-FISH. Telomere signals were detected by hybridization to FAM-(CCCTAA)₃ oligonucleotide and converted to arbitrary units of molecular equivalents of soluble fluorescence.

### T Cell Culture and Stimulation.

CD4⁺ and CD8⁺ T cells were isolated from splenocytes using MojoSort™ T cells isolation kits (Biolegend, USA). Briefly, spleens were collected in RPMI-1640 medium (Gibco, USA) as soon as mice were sacrificed. The splenocytes were isolated through spleen crushing and filtration using a 70 m strainer. Red blood cells were lysed with 1×RBC lysis buffer, splenocytes were incubated with biotin-antibody cocktails on ice for 20 mins, and streptavidin nanobeads beads were added for 5 mins, followed by pulldown of non-T cells. 6-well plates were coated by 10 μg/ml anti-mouse CD3e antibody in PBS for 2 hours at 37° C. The isolated CD4 or CD8 T cells were stimulated in CD3e antibody coated plates, together with added 2 g/ml anti-mouse CD28 antibody for 24 h. 10 ng/ml IL-2 was added at 48, 72 and 96 h and activated T cells were examined for cell proliferation using Click-iT© Plus EdU Flow Cytometry Assay Kit (ThermoFisher, USA) or harvested for gene expression analyses.

### Histological Analyses.

Fresh intestines were fixed in 10% formalin solution for 48 h, embedded in paraffin, sectioned at 5 m, and stained with hematoxylin and eosin (H&E). Mouse testes were fixed in Bouin's fixative for 3 days and similarly processed. Slides were evaluated for aberrant seminiferous tubules in a blinded fashion and at least three mice of each experimental group were analyzed.

### Hematological Analysis.

The whole blood samples were collected from submandibular veins of 3-6 months mice. Numbers of WBCs, RBCs, platelets, lymphocytes, neutrophils, and monocytes were determined on a Hemogram Analyzer (Abaxis HM5).

### FACS Analyses.

Peripheral blood, splenocytes, and femur bone marrow (BM) were collected from mice of 3-6 months. Peripheral blood was washed twice with PBS by centrifugation in the presence of heparin, followed by removing RBCs with 1×RBC lysis buffer (Thermo Fisher, USA). WBCs were counted using hemocytometers, stained with CD4, CD8, and CD19 antibodies, and analyzed by flow cytometry. Splenocytes and bone marrow cells, flushed from femurs by syringes with 20 ml PBS buffer, were similarly processed for cell counting, FACS staining, and analyses.

### Dextran Sulfate Sodium Treatment.

Female mice of 7-8 months of age were subjected to a 6-day period of 3% Dextran sulfate sodium (DSS) (Cat. 160110, MP biomedical) administration in drinking water, followed by a day of regular drinking water before tissue collection. Body weight of treated mice were monitored. For EdU labeling, 150 μl of 10 mg/ml EdU solution was injected intraperitoneally into mice two hours before sacrifice. Paraformaldehyde-fixed tissues were embedded in OCT matrix. EdU was detected using the Click-iT Plus EdU Cell Proliferation Kit (Alexa Fluor™ 594, C10639, ThermoFisher). Tissue sections were stained overnight with an anti-E-Cadherin antibody (1:250, Cat. 610181, BD Biosciences), followed by incubation with FITC-conjugated secondary antibody and Hoechst 33342 (1:2000). Images were captured using a Zeiss AXIO Image M2 microscope and processed using FIJI and Adobe Photoshop 2023.

### Example 2

Telomerase Regulation in Mice with the hmTert Allele

The hmTert gene, containing the human 5′IR (23-kb), introns 2 (11-kb) and 6 (5.5-kb) (FIG. 1A) was highly expressed in embryonic stem cells and stringently repressed upon differentiation. Tert^{h/+} mice were obtained and mated with Tert^{+/-} mice, generating mice of Tert^{h/-}, Tert^{+/-}, and Tert^{h/+} genotypes. As shown in FIG. 1B, telomerase activity was readily detected in the majority of adult tissues in a Tert^{+/-} mouse, yet it was present in a limited number of tissues in its Tert^{h/-} littermate. A direct comparison of mTert and hmTert mRNAs in a Tert^{hin/+} mouse showed that mTert mRNA was expressed in most organs, whereas high hmTert mRNA expression was found only in thymus (FIG. 1C). Relatively low levels of hmTert mRNA were detected in testis and ovary, and very low levels in intestine and spleen. The hmTert mRNA expression pattern was virtually identical to that of the hTERT mRNA in human tissues (FIG. 1D), indicating that the developmental regulation of the hmTert gene recapitulated that of hTERT in humans.

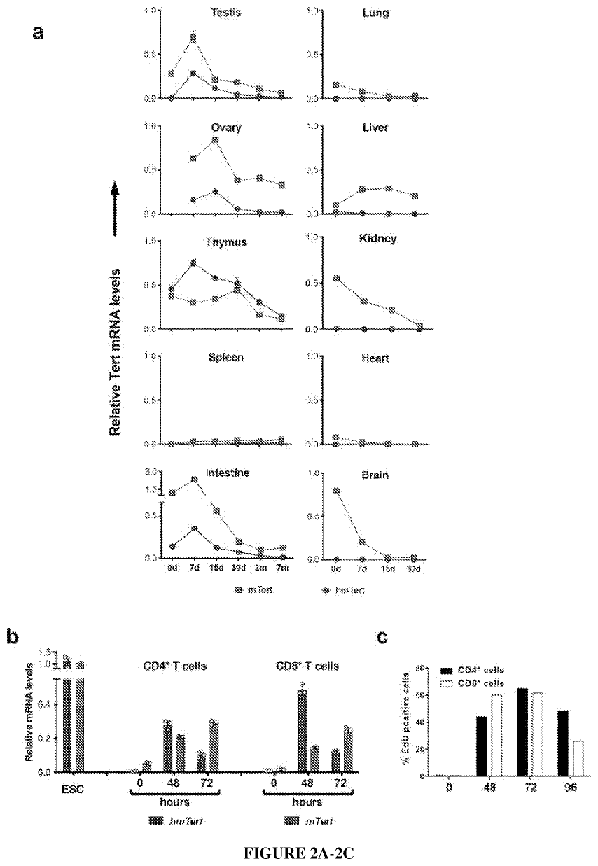

During postnatal development, hmTert expression in testis, ovary, thymus, and intestine was more pronounced within the first two weeks of newborns and decreased with age in young mice (FIG. 2A). Only thymus maintained significant hmTert expression. By comparison, mTert expression was high in most organs of newborns and decreased upon development, except for ovary, liver, and spleen, where high and low mTert mRNA levels were maintained in adults, respectively.

It was reported that hTERT expression progressively declined during T cell differentiation and T cells rapidly upregulated hTERT mRNA to support robust cell division and differentiation. Consistently, resting mouse T cells expressed little hmTert mRNA and its level increase dramatically in CD4⁺ and CD8⁺ T cells following stimulation by CD3/CD28 antibodies for 48 and 72 hours (FIG. 2B). mTert RNA was readily detected in resting T cells and its level also increased during T cell activation. The increase of hmTert and mTert RNAs correlated with EdU incorporation, and thus cell proliferation, in T cells (FIG. 2C).

### Example 3

Telomere lengths in Tert^{+/-}, Tert^{h/-}, and Tert-KO mice

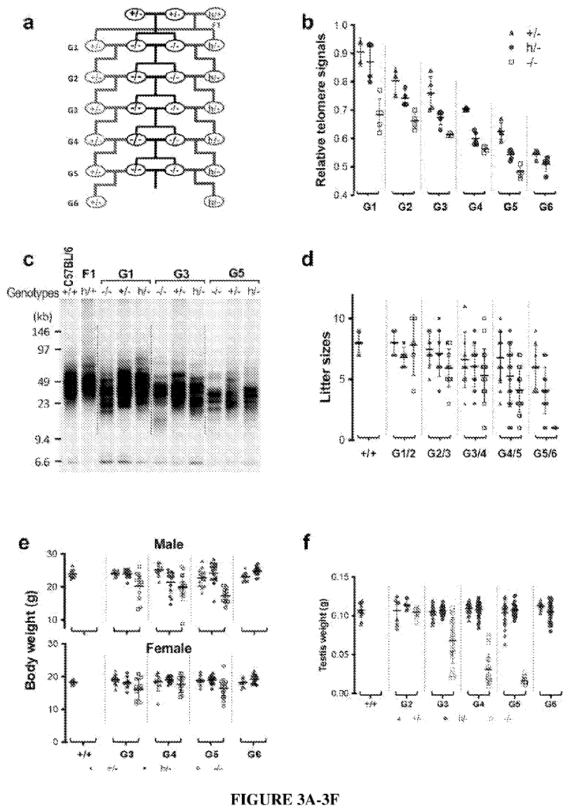

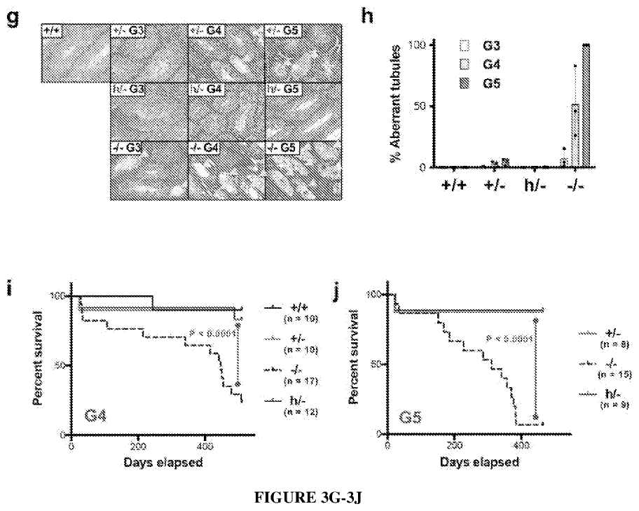

C57BL/6J mice have an average telomere length of approximately 50 kb. To reduce telomere length in mice with hmTert genes, Tert^{+/h} F1 mice were crossbred to Tert^{+/-} mice to produce Tert^{h/-} and Tert^{+/-} offspring, which were then bred with successive generations of Tert^{-/-} mice (FIG. 3A). Telomere length of these mice were examined by flow cytometry following fluorescence in situ hybridization (Flow-FISH) and telomere restriction fragment (TRF) analysis by Southern blotting (FIG. 3B, FIG. 3C). As the breeding of Tert^{-/-} mice resulted in shortened telomeres, similar decreases in telomere length were observed in both Tert^{+/h} and Tert^{h/-} mice over successive generations. In each generation, Tert^{h/-} mice had shorter telomeres on average compared to their Tert^{+/-} counterparts. In G6 Tert^{h/-} mice, the average telomere length dropped to roughly 50% of the length in wildtype mice, or ~25 kb. The litter sizes of Tert^{+/-}×Tert^{-/-} and Tert^{h/-}×Tert^{-/-} mattings declined over generations, while the litter sizes of homozygous Tert^{-/-} incrosses decreased even more dramatically (FIG. 3D). In several attempts, G6 Tert^{-/-} mice produced only one offspring which died prematurely, ultimately ending this breeding strategy. However, the reduction in telomere length in Tert^{+/-} and Tert^{h/-} mice did not negatively impact their overall health and well-being, as evidence by their normal body weight in G6 mice (FIG. 3E).

### Example 4

Maintaining Testis Homeostasis by the hmTert Gene

Due to the impact of shorter telomeres on mouse fertility, we analyzed the testes of these mice. As illustrated in FIG. 3F, FIG. 3G, FIG. 3H, Tert^{-/-} mice showed testicular atrophy as well as a progressive loss of germ cells in seminiferous tubules starting from G3 mice and worsening in G4 and G5 mice, as previously reported in telomerase-deficient mice. Tert^{+/-} mice also exhibited a low level of testicular defects in G4 and G5, but such defects were absent in Tert^{h/-} mice. These data indicate that both mTert and hmTert genes help preserve germ cells in the testis, with hmTert showing a slight advantage in preventing germ cell loss.

### Example 5

Sustaining Mouse Lifespan by the hmTert Gene

Previous studies have shown that telomere deficiency can impact mouse survival and lifespan. Our results align with those findings, as shown in FIGS. 3I, 3J, G4 and G5 Tert^{-/-} mice had a significantly shortened lifespan, with median survival of approximately 440 and 320 days, respectively. However, the majority of wildtype Tert^{+/+}, G4 and G5 Tert^{+/-} and Tert^{h/-} mice lived past 500 days, indicating that the presence of a mTert or hmTert gene could sustain longevity even when telomeres were relatively short.

### Example 6

Rescusing Telomere Dysfunction in Tert^{-/-} Mice by the hmTert Gene

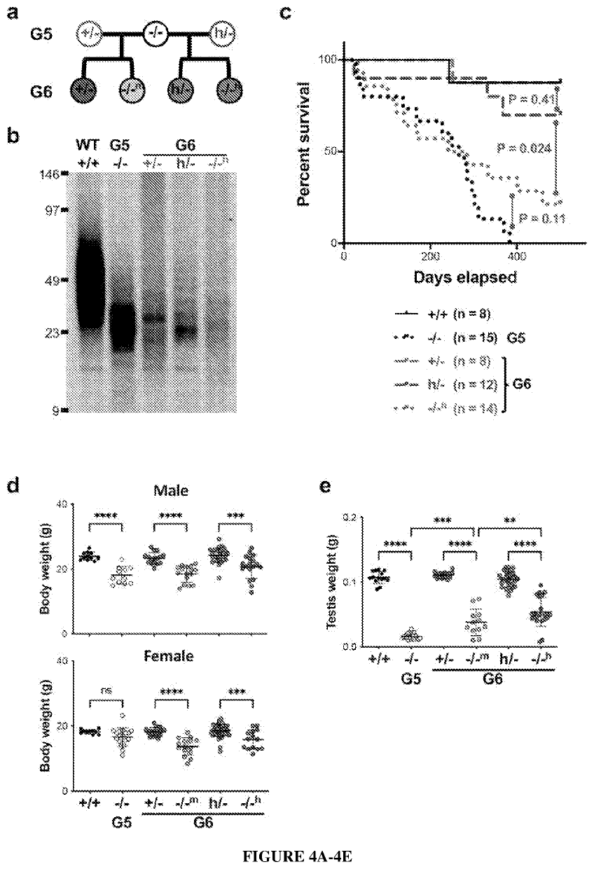

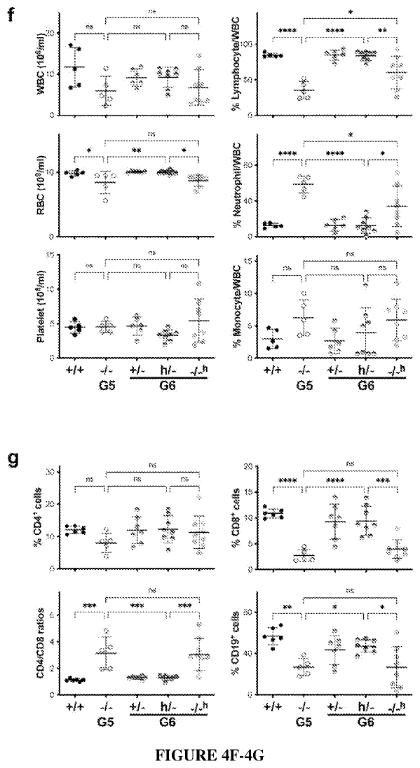

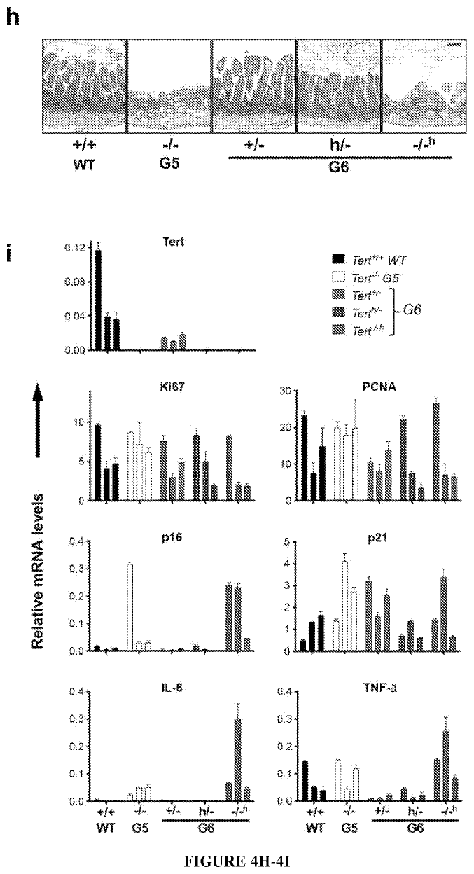

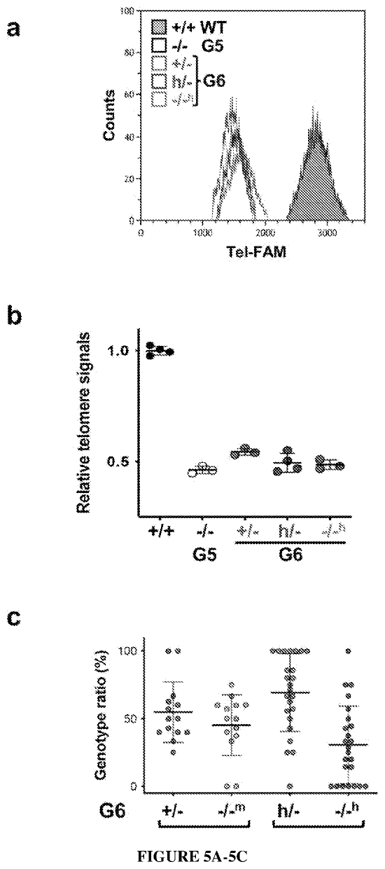

Offspring inherit not only parents' genotypes but also the lengths of their telomeres. G5 Tert^{-/-} mice displayed severe telomere dysfunction, evidenced by tissue dystrophy, reduced body weight, and a shortened lifespan. The next generation, denoted as G6 Tert^{-/-} and G6 Tert^{-/-h}, was generated by crossing G5 Tert^{-/-} mice with G5 Tert^{+/-m} and Tert^{h/-} mice, respectively (FIG. 4A). Examination of telomere length through TRF and Flow-FISH analyses indicated that the average telomere lengths in G6 Tert^{h/-} and Tert^{+/-} mice were comparable to, or slightly longer than, those of their G5 Tert^{-/-} parents and G6 Tert^{-/-} siblings (FIG. 4B, FIG. 5A, and FIG. 5B). However, despite these similarities, G6 Tert^{h/-} and Tert^{+/-} mice exhibited significantly extended lifespans compared to their G5 Tert^{-/-} mice (FIG. 4C). Notably, while all G5 Tert mice died within 383 days, three out of 14 G6 Tert^{-/-h} mice survived beyond the entire 460-day experimental period. This resilience could be attributed to the inheritance of short yet functional telomeres from their G5 Tert^{h/-} parents. Taken together, these findings suggest that the hmTert allele in Tert^{h/-} mice has the capacity to restore the shortest and likely most impaired telomeres, even in the context of their overall short average telomere length.

Further examination of genotypes among G6 progeny indicated that fewer G6 Tert^{-/-} offspring were born compared to their Tert^{+/-} and Tert^{h/-} siblings (FIG. 5C), indicating that some Tert^{-/-} offspring died during prenatal development. Although G6 Tert^{-/-m} and G6 Tert^{h/-h} mice had reduced body and testis weights compared to their Tert^{+/-} and Tert^{h/-} littermates, respectively, their testes weighed significantly more than their G5 Tert^{-/-} parents (FIGS. 4D-4E). In addition, G6 Tert^{-/-h} mice had a slightly increased testis weight compared to G6 Tert^{-/-m} mice, suggesting that the hmTert gene was functionally similar to, or somewhat better than, the mTert gene for rescuing testicular defects. Overall, G6 offspring showed better general health compared to their G5 Tert^{-/-} parents, demonstrating the telomere function-restoring capacities of both the hmTert and mTert genes within a single generation.

### Example 7

hmTert Function in Immune System

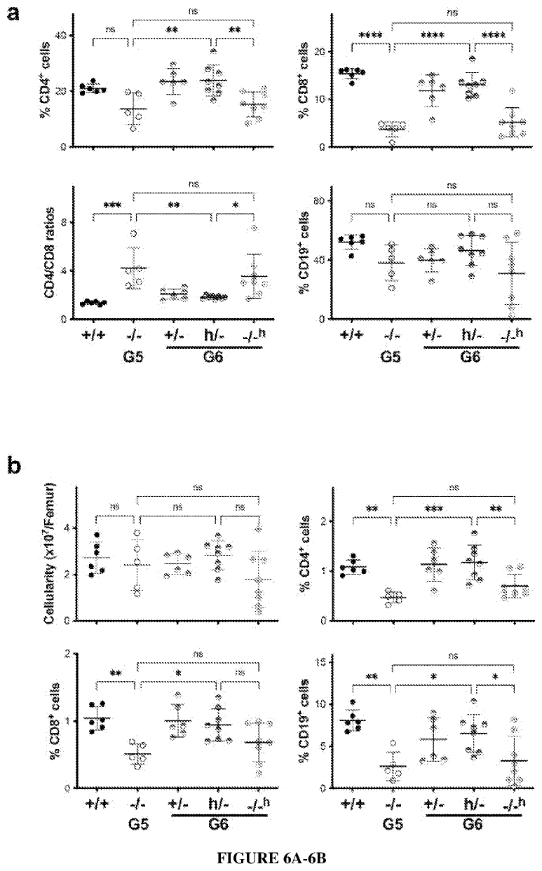

Previous studies have shown that hematopoietic cells' proliferative capacity was compromised in telomerase-deficient mice, and human short telomere syndromes cause anemia, decreased erythropoiesis, and T cell immunodeficiency. Consistent with earlier reports, peripheral blood from G5 Tert^{-/-} mice exhibited a slight decrease in white blood cell (WBC) counts, a statistically significant reduction in red blood cell (RBC) counts, and normal platelet numbers (FIG. 4F). Among WBCs, there was a marked decrease of lymphocytes observed in G5 Tert^{-/-} mice, accompanied by corresponding increases in the ratios of neutrophils and monocytes. Both G6 Tert^{+/-} and Tert^{h/-} mice had blood cell counts similar to wildtype mice. The lymphocyte and neutrophil cell percentages within WBCs of G6 Tert^{-/-h} mice were between those of wildtype mice and their G5 Tert^{-/-} parents. G5 Tert^{-/-} mice had a somewhat reduced percentage of CD4⁺ T cells and dramatically reduced CD8⁺ T cells, leading to a significant increase of the CD4/CD8 T cell ratio (FIG. 4G). Additionally, CD19⁺ B cells decreased in G5 Tert^{-/-} mice. All these cell counts in G6 Tert^{+/-} and Tert^{h/-} mice were restored to the levels found in wildtype mice. Further analyses of T and B cell counts in spleen and bone marrow revealed similar changes in G5 Tert mice and Tert^{+/-} and Tert^{h/-} offspring (FIGS. 6A and 6B). In short, our data indicate that the hmTert gene effectively rescued blood cell defects in G5 Tert^{-/-} mice within one generation.

### Example 8

hmTert Function in Small Intestine

The gastrointestinal tract is another high proliferation tissue that is affected by telomere dysfunction. Depletion of the intestinal epithelial crypts and severe villus atrophy were observed in small intestines of older G5 Tert^{-/-} (>8 months), but not in G6 Tert^{+/-} and Tert^{h/-} mice (FIG. 4H). The intestinal lesions probably contributed to the loss of body weight and overall poor health of Tert^{-/-} animals due to decreased nutritional absorption.

The intestinal defects are likely a consequence of cell cycle inhibition and cellular senescence induced by telomere dysfunction. Therefore, the expression of genes involved in cell proliferation was examined. Tert mRNA was readily detected in the intestines of Tert^{+/+} and Tert^{+/-} mice, but not in those of Tert^{h/-} or Tert^{-/-} mice, confirming that the hmTert gene is strictly regulated in adult tissues (FIG. 4I). Markers of cell proliferation, Ki-67 and PCNA, and the cell cycle inhibitor p21 were found in the intestine of all genotypes. Senescence-associated genes, p16^{lnk4a} and IL-6, were upregulated in G5 Tert^{-/-} and G6 Tert^{-/-h} mice, but not in any mice with mTert or hmTert genes. TNF-α, another pro-inflammatory cytokine secreted by senescent cells, appeared to be expressed in mouse intestines of all genotypes. Therefore, our data suggest that cellular senescence occurred in Tert^{-/-} intestines with significant telomere dysfunction but was suppressed by the presence of the hmTert gene in this tissue.

### Example 9

### Telomere Shortening During Intercrosses of Tert^{h/-} Mice

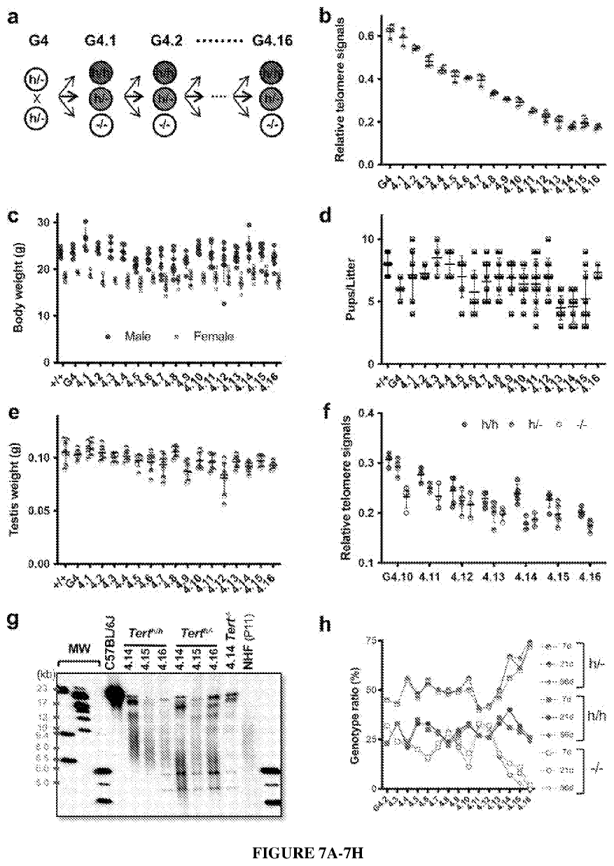

Despite its restricted expression in adult tissues, the hmTert gene rescued telomere dysfunction in G5 Tert^{-/-} mice with relatively short average telomeres of ~25-kb. Our next objective was to determine the telomere length setpoint influenced by the hmTert gene. To this end, G4 Tert^{h/-} mice were continuously intercrossed for 16 generations, from G4.1 to G4.16 (FIG. 7A). Using Flow-FISH, we monitored telomere length of Tert^{h/-} mice in splenocytes at each generation. As depicted in FIG. 7B, the average telomere length of Tert^{h/-} mice decreased from 60% to 18% of that observed in wildtype mice from G4 to G4.14, eventually stabilizing at 18-19% in the last three generations (G4.14 to G4.16). Throughout the breeding generations, both male and female Tert^{h/-} mice maintained body weights similar to those of wildtype mice (FIG. 7C). During this process, litter sizes varied, but were largely maintained (FIG. 7D). Male mice also maintained their testis weight (FIG. 7E). FIG. 7F compares the average telomere length of all three genotypes in each generation, from G4.10 to G4.16. It demonstrates that Tert^{h/h} mice in general had longer telomere than their Tert^{h/-} siblings, and that Tert^{-/-} mice consistently exhibited the shortest telomeres across generations. Overall, our data indicated that average mouse telomeres could be shorten to below 10 kb without affecting their overall health, at least at a young age, as long as they have the hmTert gene.

Telomere length in later generations of mice was also verified using TRF analysis (FIG. 7G). Two types of telomeres were found in these mice: discrete bands of variable sizes and intensities between 15 and 20 kb, and shorter human-like telomere smears. The average lengths of the telomere smear were 8-9 kb in the G4.16 Tert^{h/h} mice and about 7 kb in the Tert^{h/-} mice. In a G4.14 Tert^{-/-} mouse, the telomere smear was much less apparent. FIG. 7H shows that, from G4.2 to G4.12, approximately 50% of total born mice were of heterozygous Tert^{h/-} genotype, while homozygous Tert^{h/h} and Tert^{-/-} mice each accounted for about 25% of total progeny, following Mendelian genetics. However, there was a sharp decline in the numbers of Tert^{-/-} mice born from G4.13 to G4.16. The few Tert^{-/-} mice that were born were small and die at young ages. These data indicated that short telomeres in late-generation Tert^{-/-} embryos could no longer sustain mouse development.

### Example 10

### Maintaining Stable Human-Like Telomeres in Homozygous Tert^{h/h} Mice

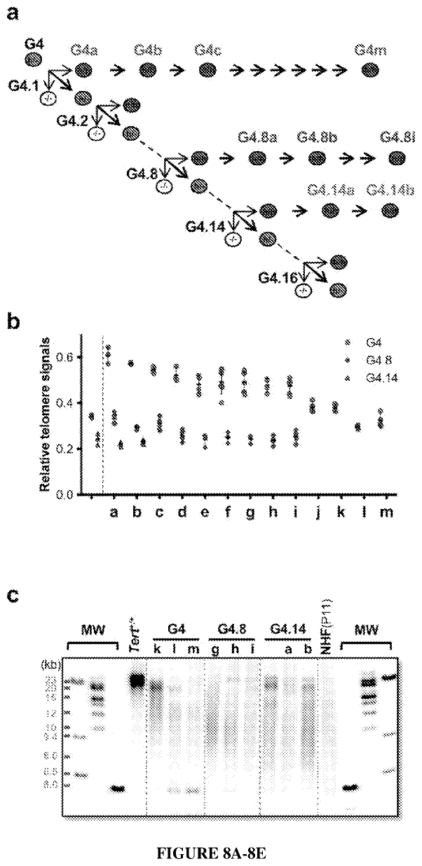

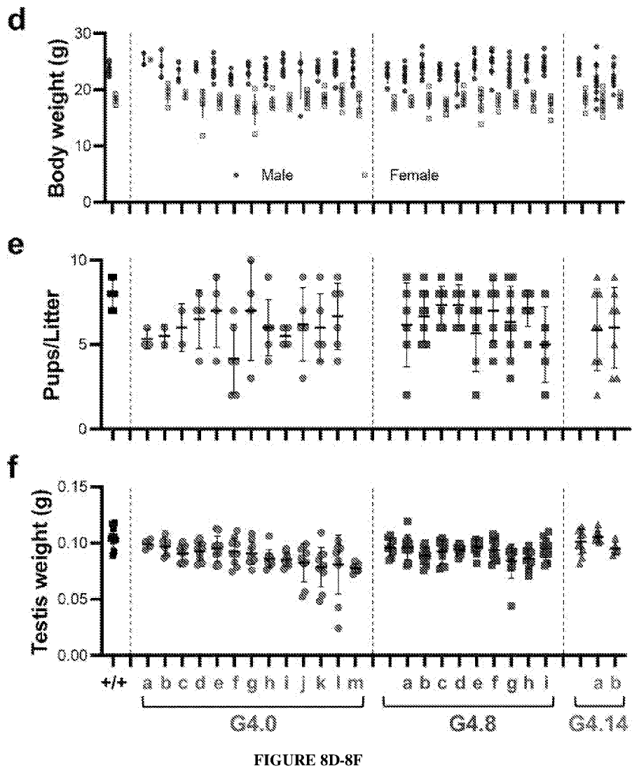

To assess the stability of short telomeres in mice with the hmTert genes, homozygous Tert^{h/h} offspring from G4, G4.8, and G4.14 were incrossed for 13, 9, and 2 generations, respectively (FIG. 8A). Average telomere lengths in their progeny were measured by Flow-FISH. The results, depicted in FIG. 8B, revealed a gradual decrease in telomere length across successive generations. In G4 Tert^{h/h} mice, telomeres decreased from 60% to 30%, while in G4.8 Tert^{h/h} mice, the decline went from 34% to 24%. G4.14 Tert^{h/h} mice exhibited telomere lengths approximately 24% of the wildtype, and their offspring maintained telomeres at 22-23% of wildtype length during two successive generations of incrossing. These findings indicate that telomere length in Tert^{h/h} mice stabilized at a shortened but consistent ranges of 21-24% of wildtype mice, equivalent to an average telomere length of 10-12 kb, similar to reported leukocyte telomere lengths of 9.5±0.7 kb and 10-11 kb in newborn humans. Additionally, TRF analysis confirmed the presence of discrete telomere bands between 15-20 kb and a human-like telomere smear (FIG. 8C). For G4.8(g-i) and G4.14(a-b) mice, the average lengths of telomere smears were 9-10 kb. Regardless of having shortened telomeres, these mice exhibited good health, as demonstrated by stable body weight, litter sizes, and testis weight (FIGS. 8D-8F).

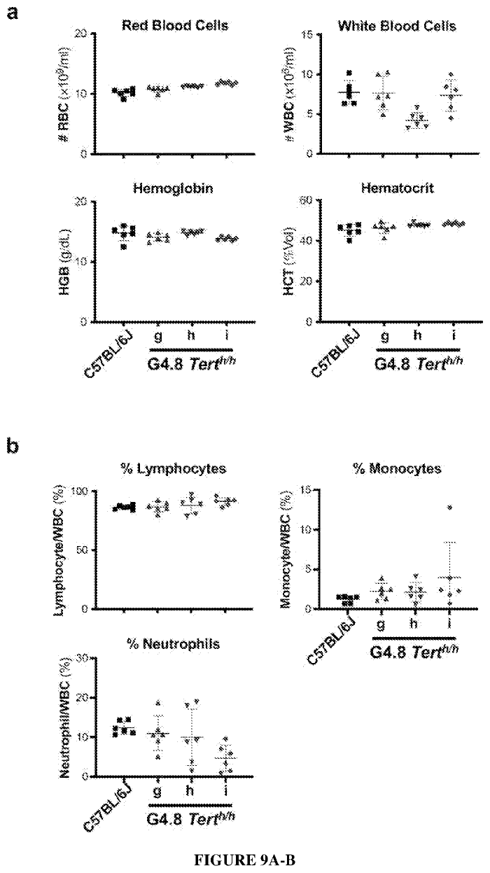

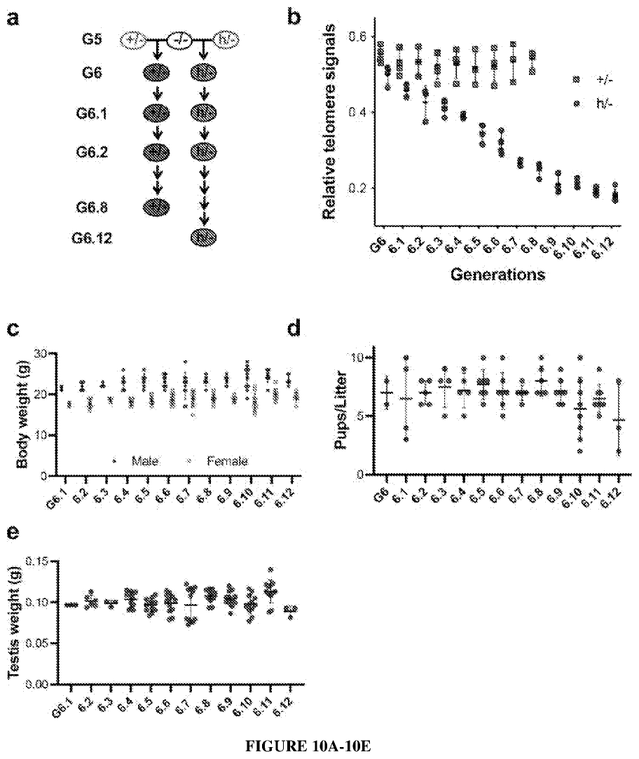

To further determine the health status of Tert^{h/h} mice with human-like telomeres, we performed hematology analysis on peripheral blood samples from G4.8g, h, and i mice aged 2-3 months. As shown in FIGS. 9A and 9B, the result indicated normal red blood cell counts, hemoglobin, and hematocrit in all groups of mice. Although the average WBC count in G4.8 h was lower than those of G4.8g, G4.8i, and wildtype mice, it still fell within the normal WBC range of physiological data for C57BL/6J mice published by the Jackson Laboratory. Within WBC populations, all groups of mice displayed normal percentages of lymphocytes, monocytes, and neutrophils, further supporting the notion that Tert^{h/h} mice with human-like telomeres maintain good health at early stages of their life.

### Example 11

Comparing hmTert and mTert Gene Functions

In order to directly compare the functions of the hmTert and mTert genes and establish a new line of Tert^{h/h} mice, we conducted intercrosses using G6 Tert^{h/-} and Tert^{+/-} mice (FIG. 10A). Consistent with our previous findings, telomere length in G6 Tert^{h/-} mice decreased from 55% to 18-19% of wildtype telomere length in C57BL/6J mice across 12 successive intercrosses (FIG. 10B). In contrast, the average telomere lengths in G6 Tert^{+/-} mice remained stable at 55% of wildtype telomere length over eight intercrosses. This result aligned with a previous study in which Tert^{+/-} mice were intercrossed for 17 generations, stabilizing their average telomere length at approximately 50% of wildtype telomeres. Importantly, G6 Tert^{h/-} mice exhibited consistent litter sizes and development with appropriate body and testis weights across the entire breeding process (FIGS. 10C-10E). Therefore, our data indicate that the Tert loci play a crucial role in regulating telomere length homeostasis, and the hmTert gene genetically determined short telomeres in mice.

### Example 12

### Decreased In Vivo Cell Proliferation Capacity in Tert^{h/h} Mice

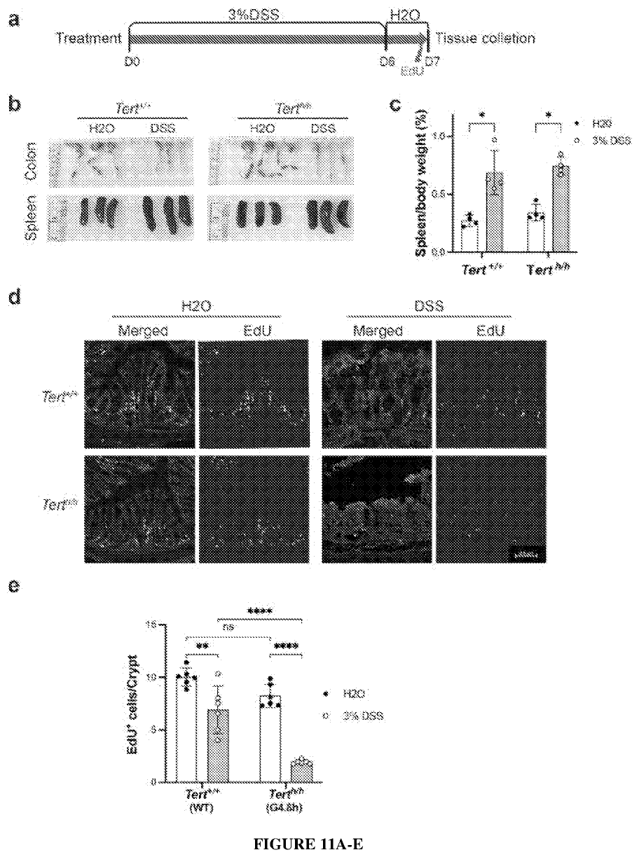

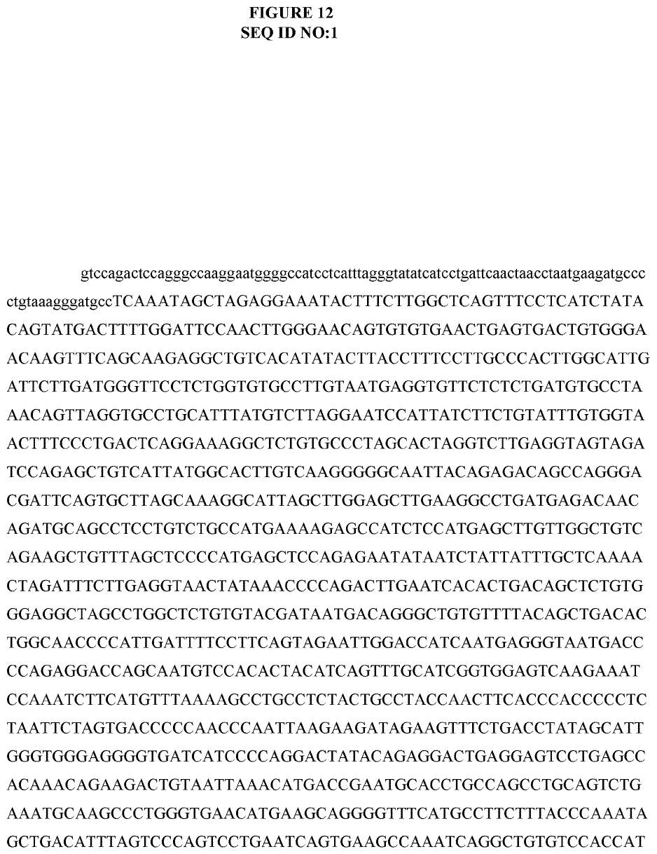

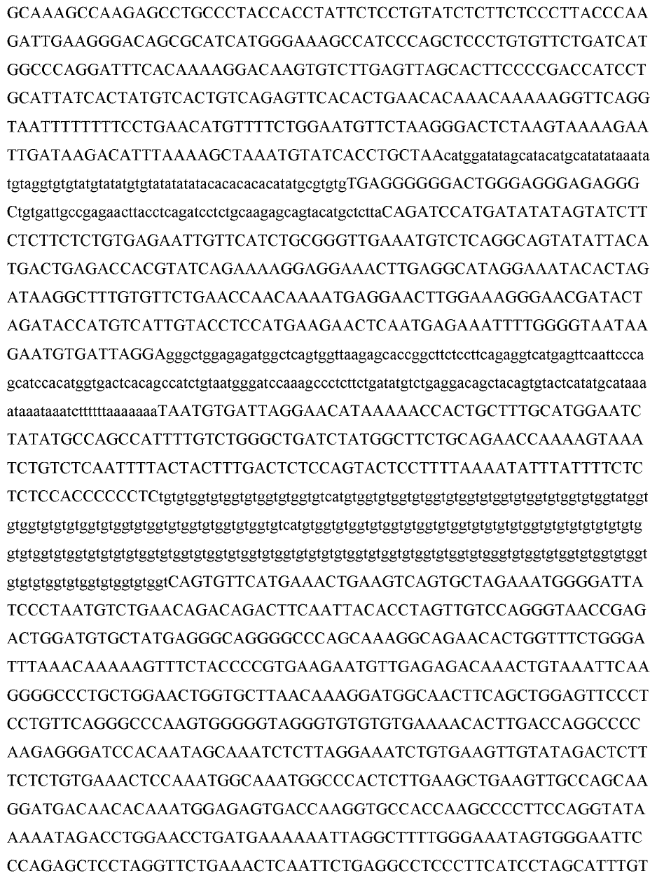

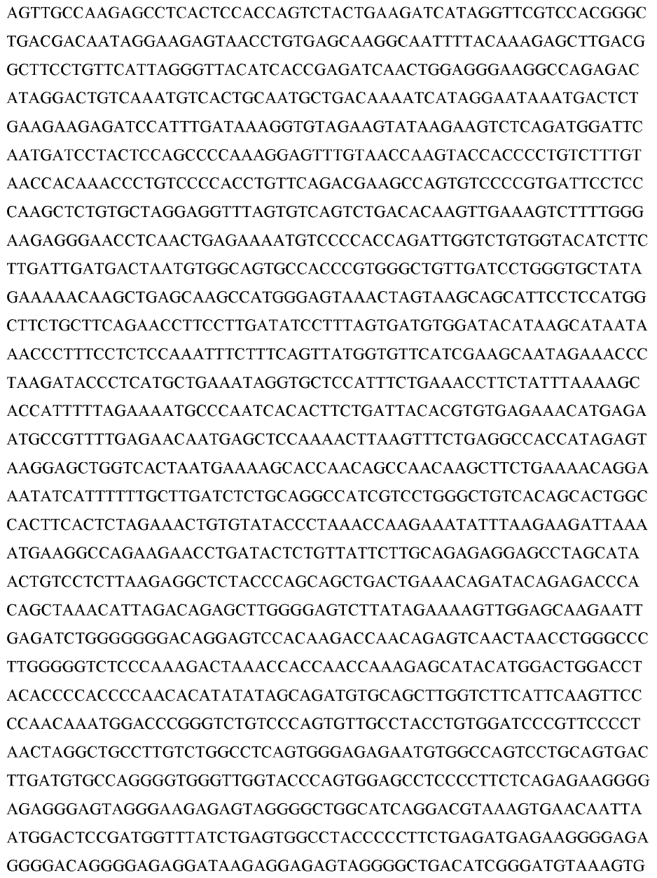

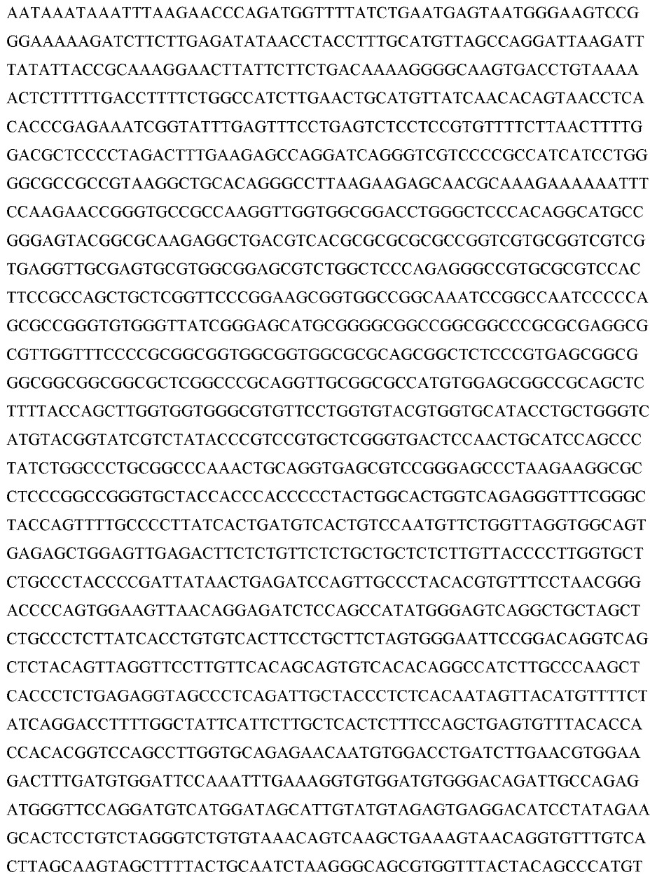

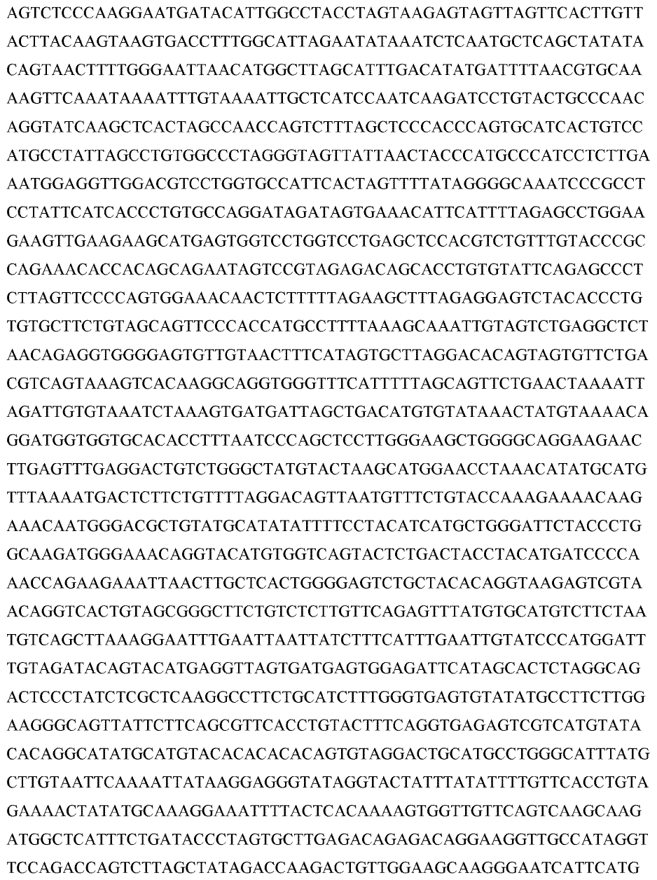

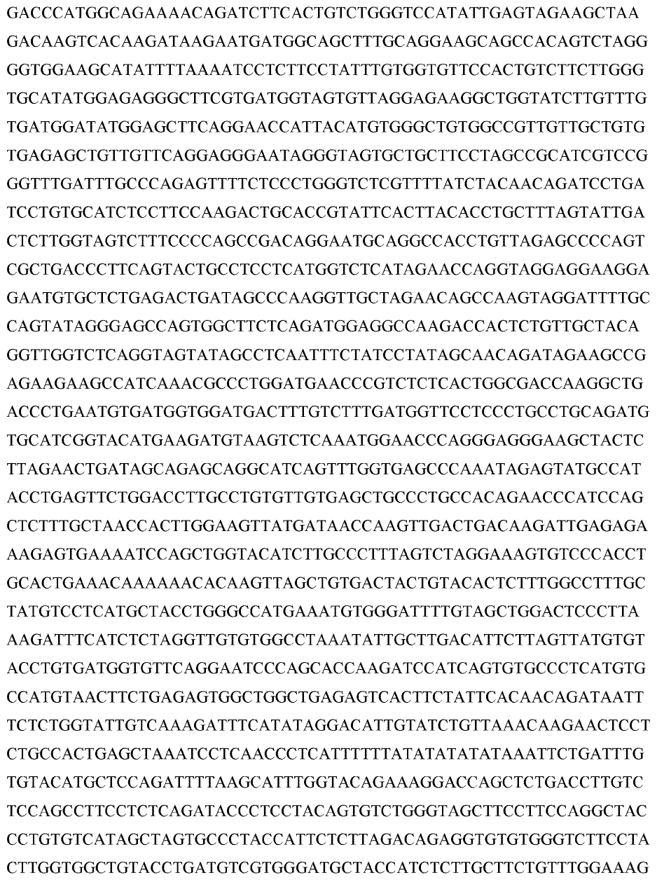

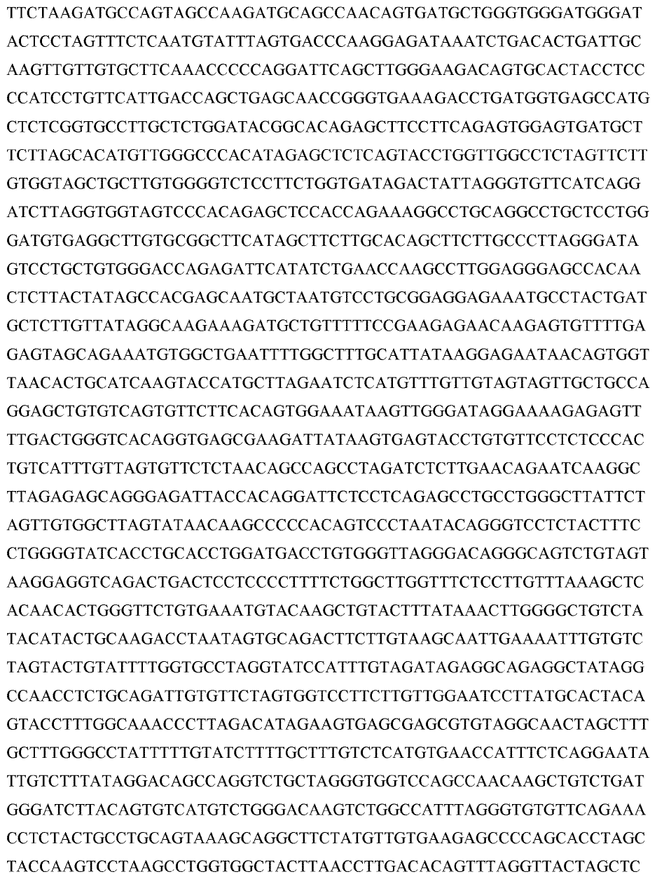

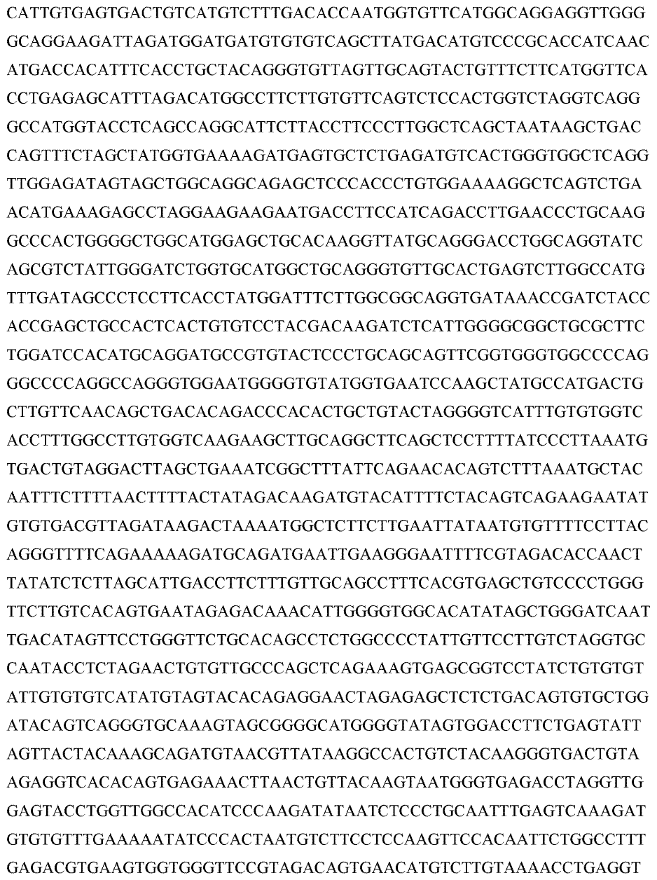

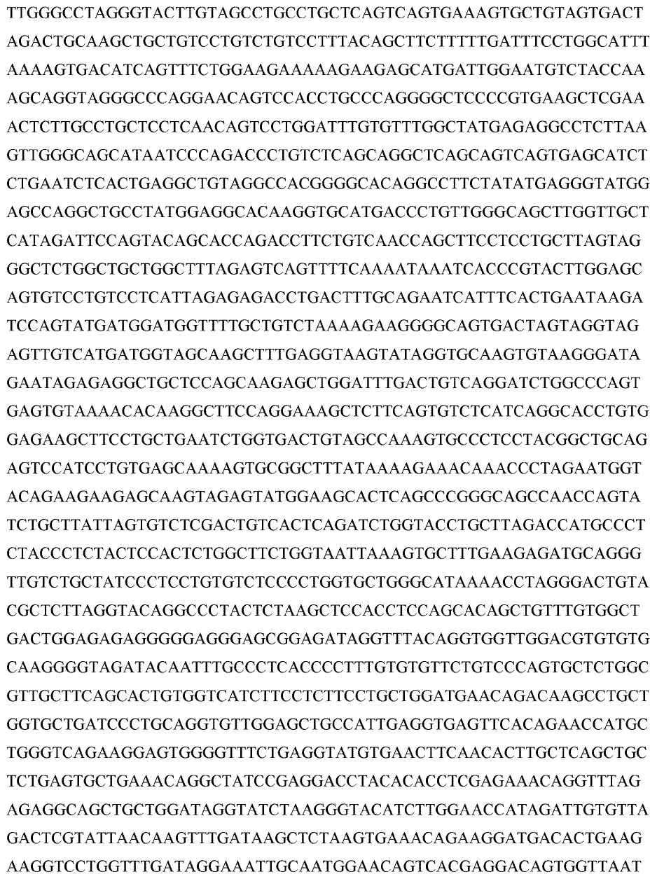

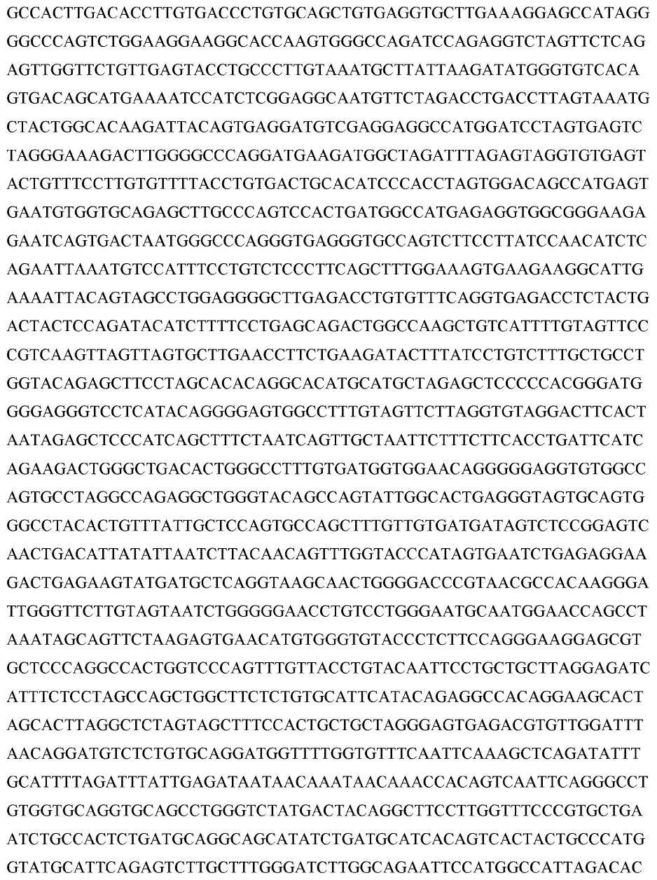

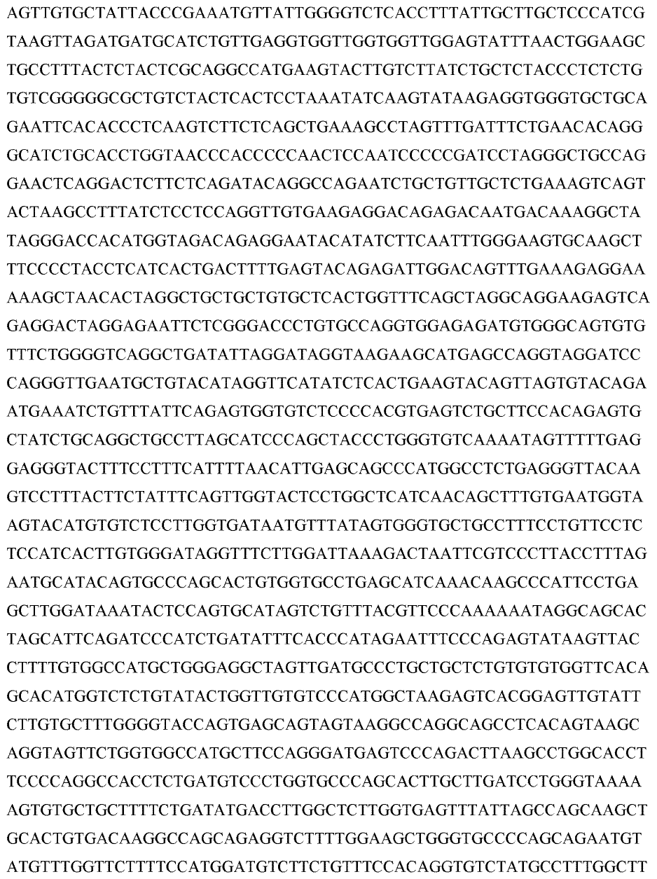

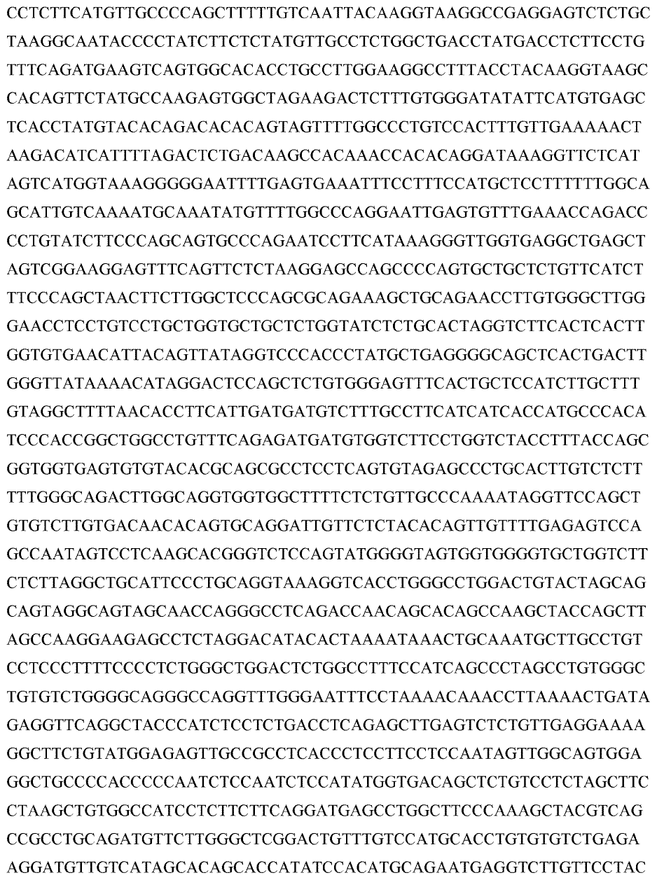

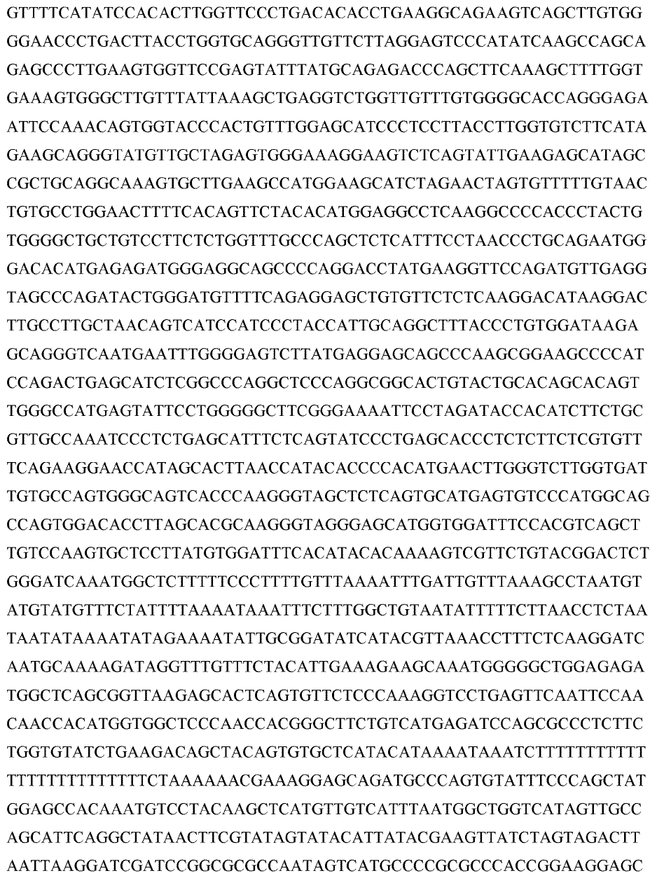

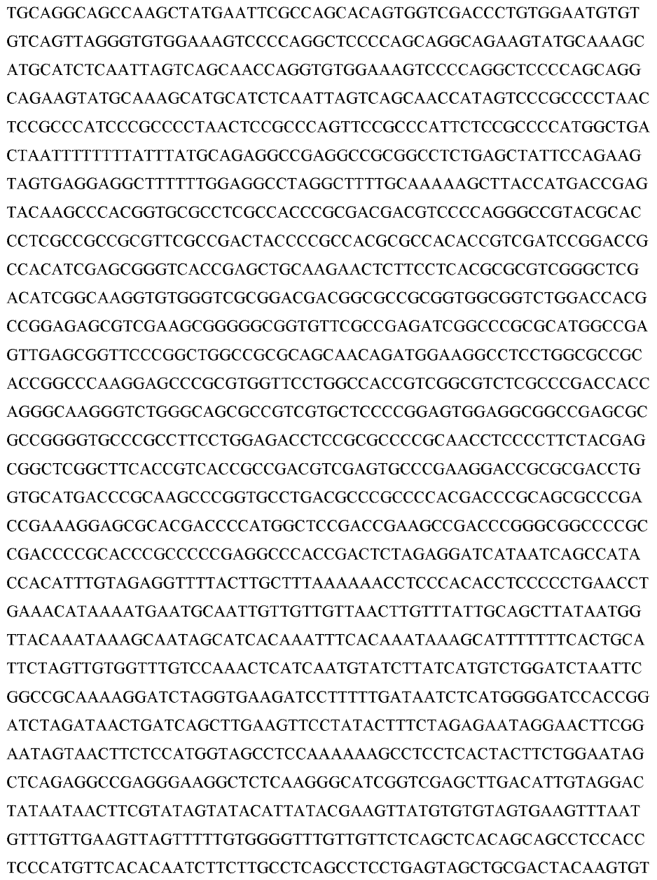

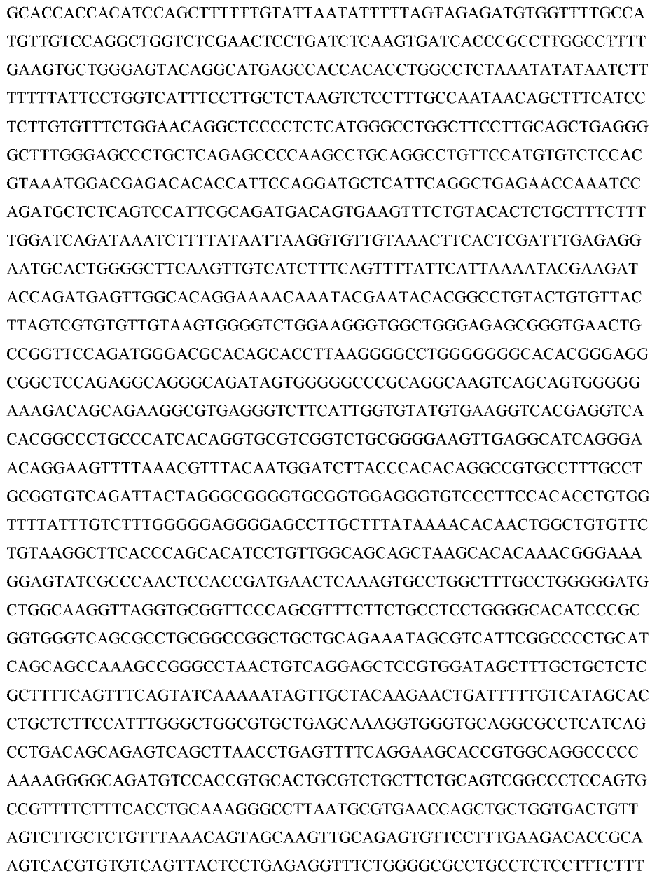

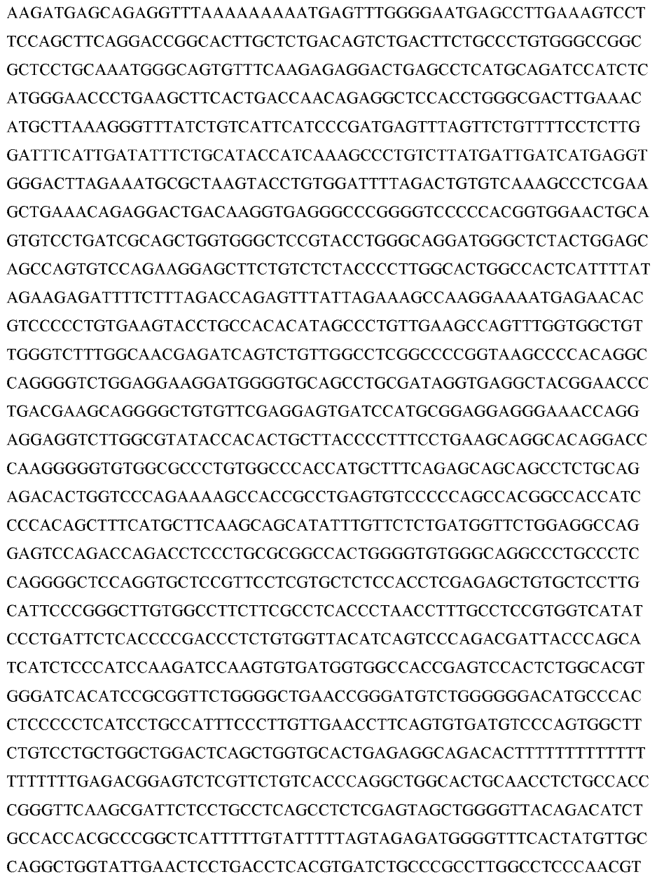

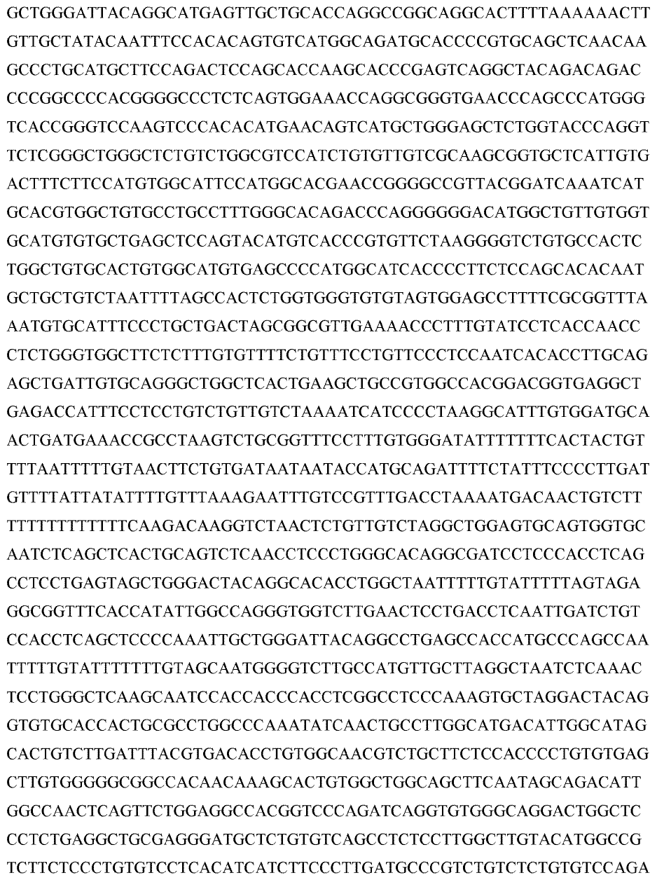

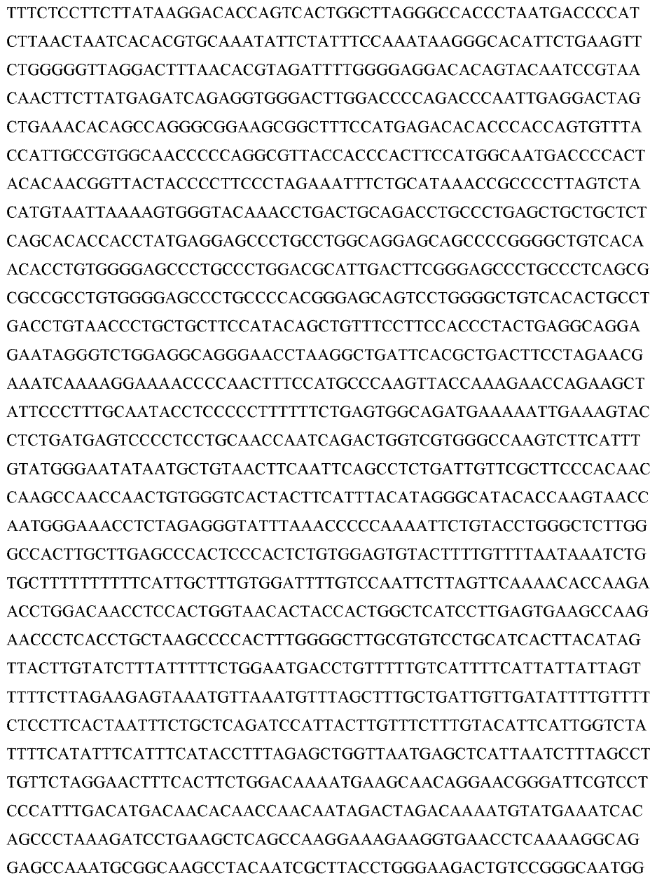

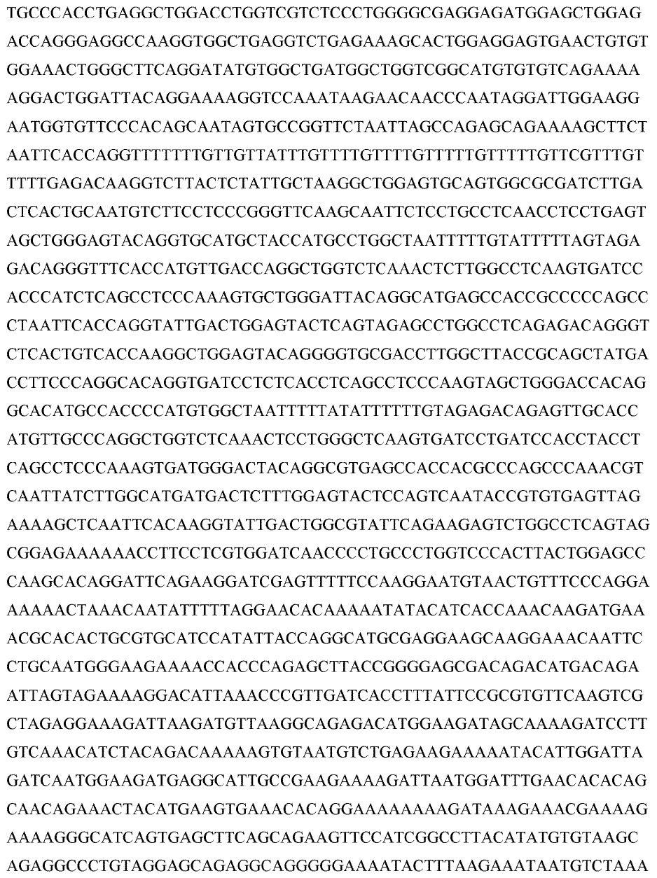

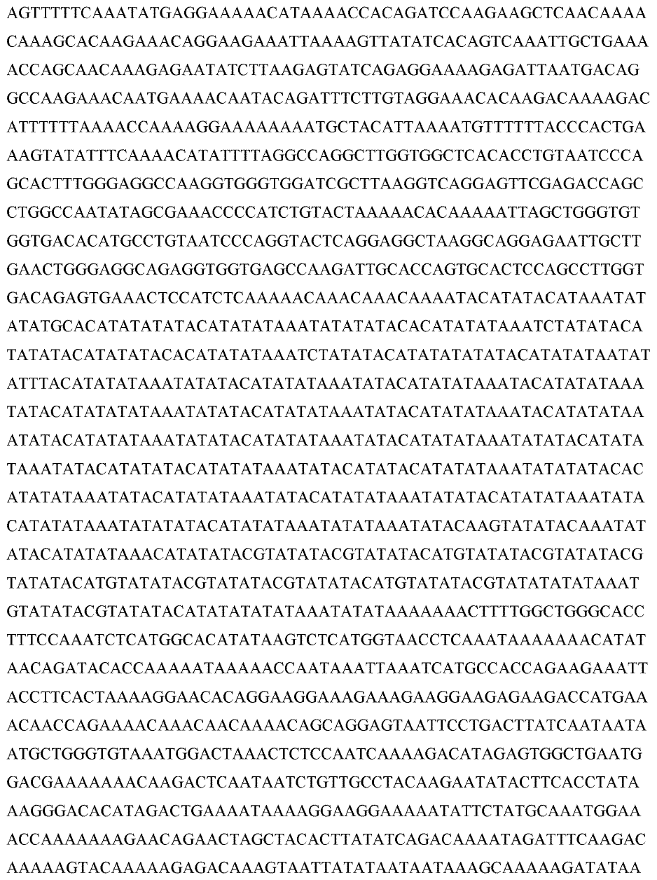

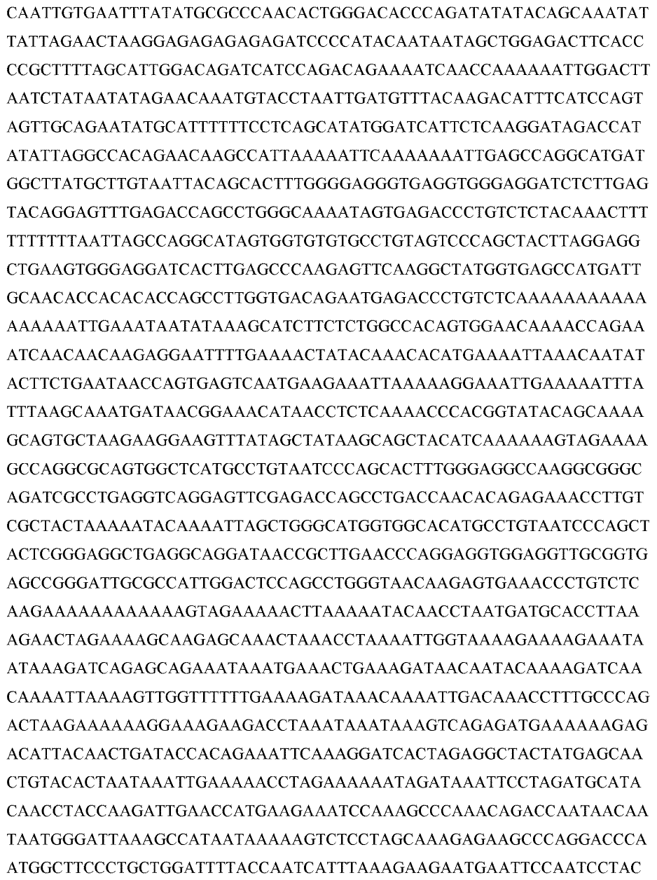

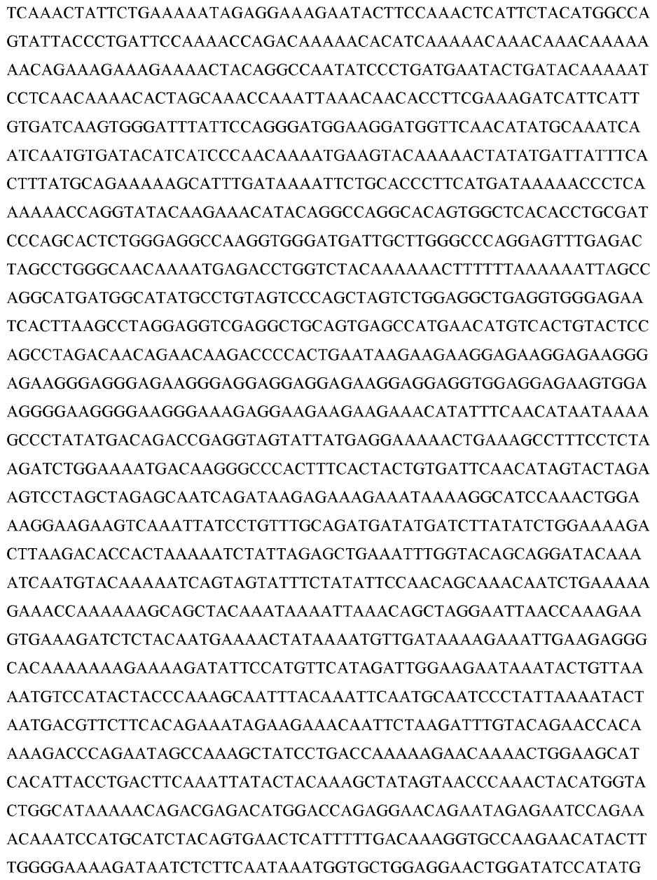

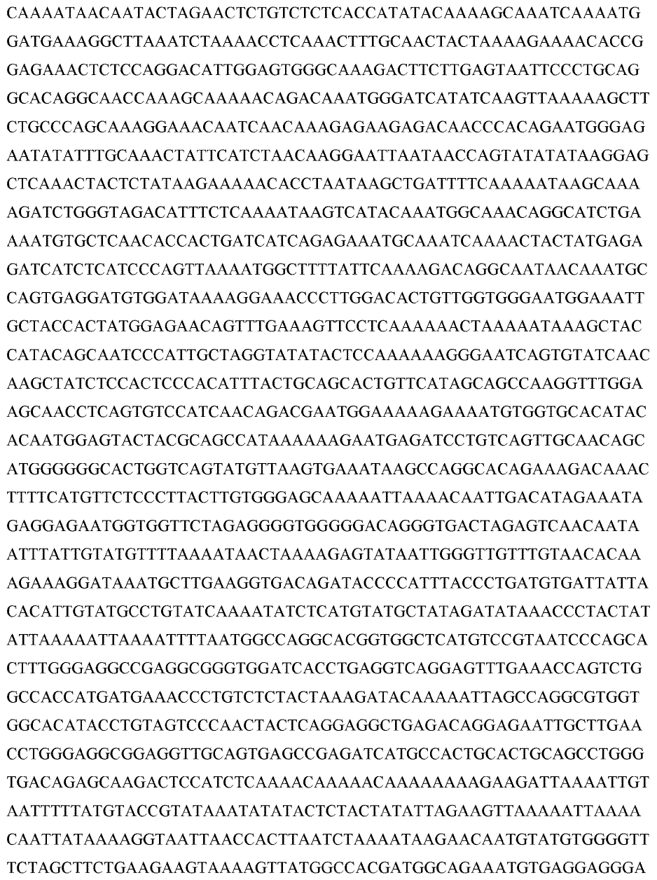

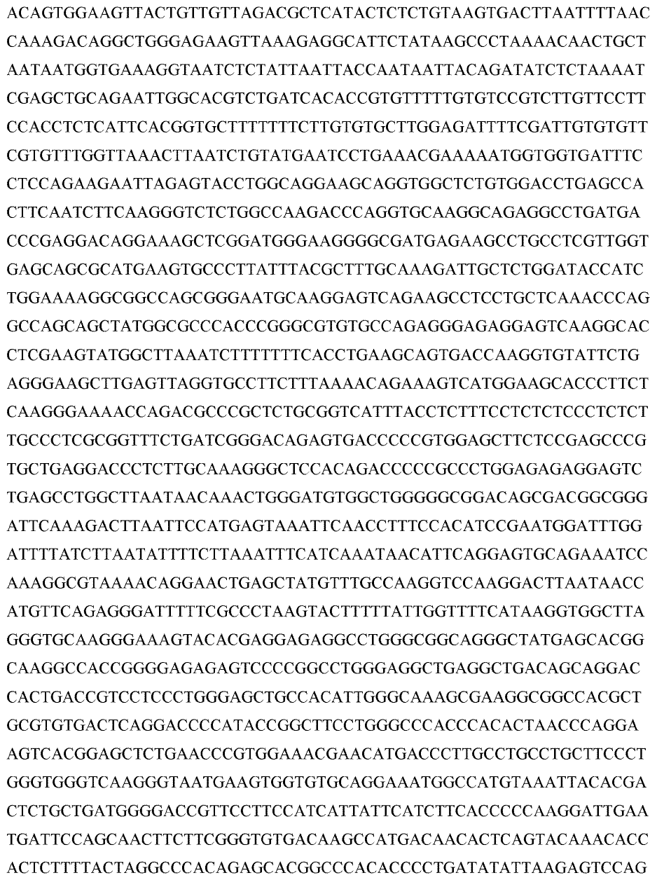

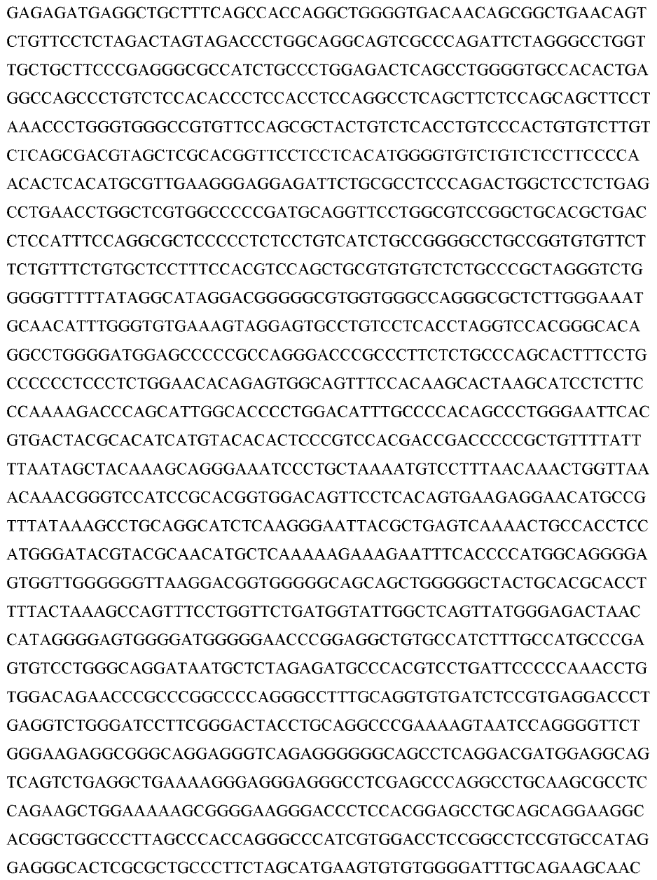

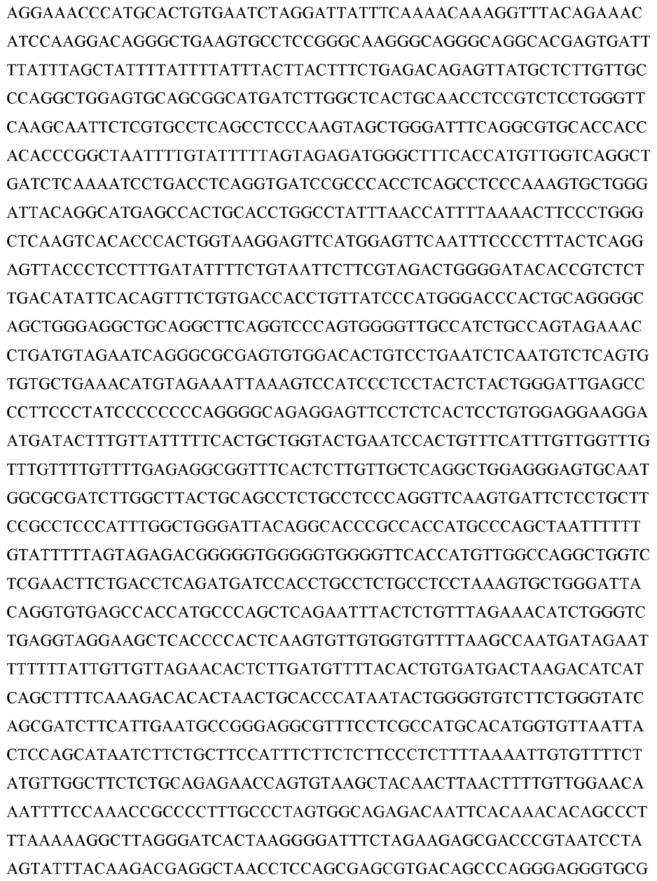

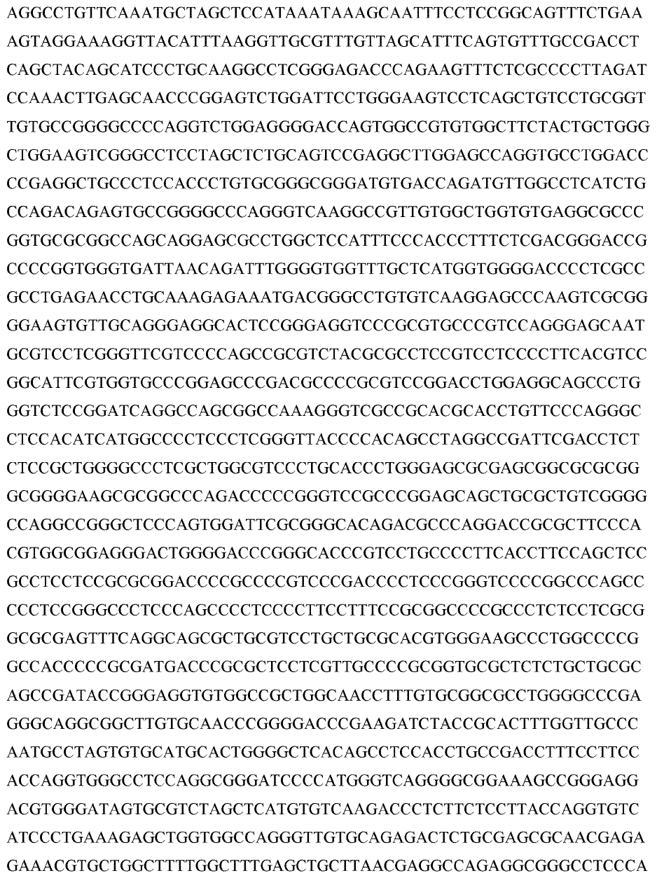

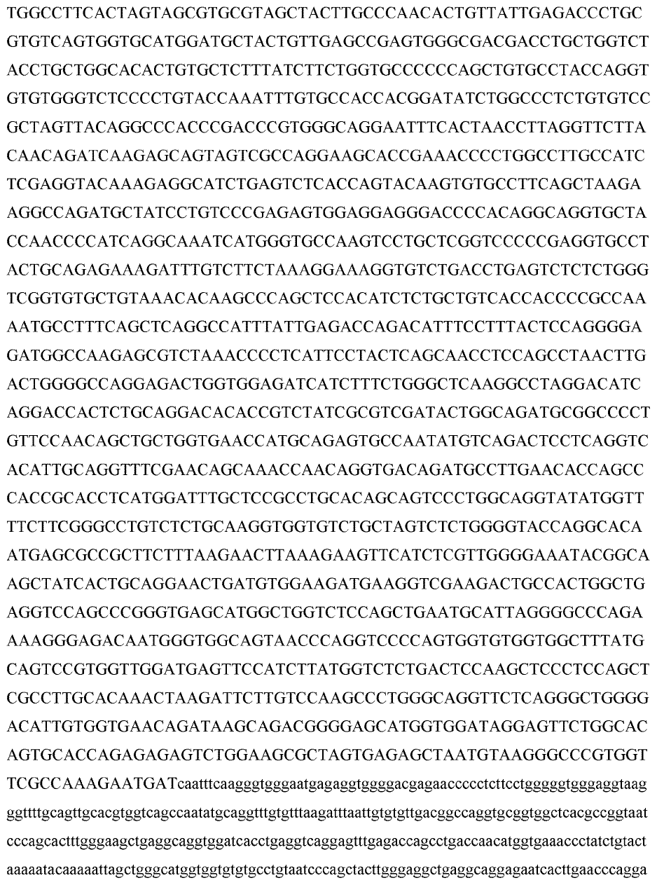

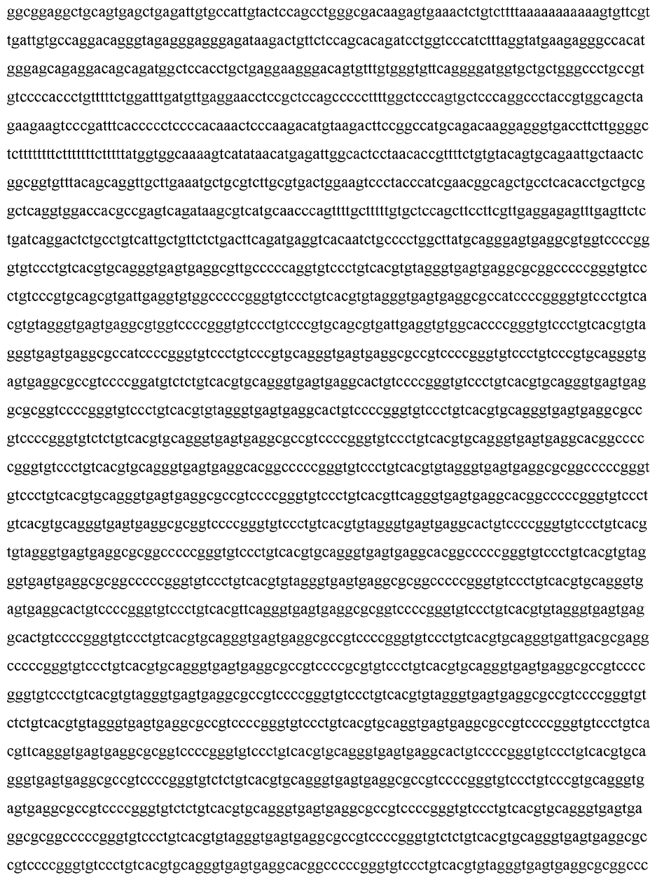

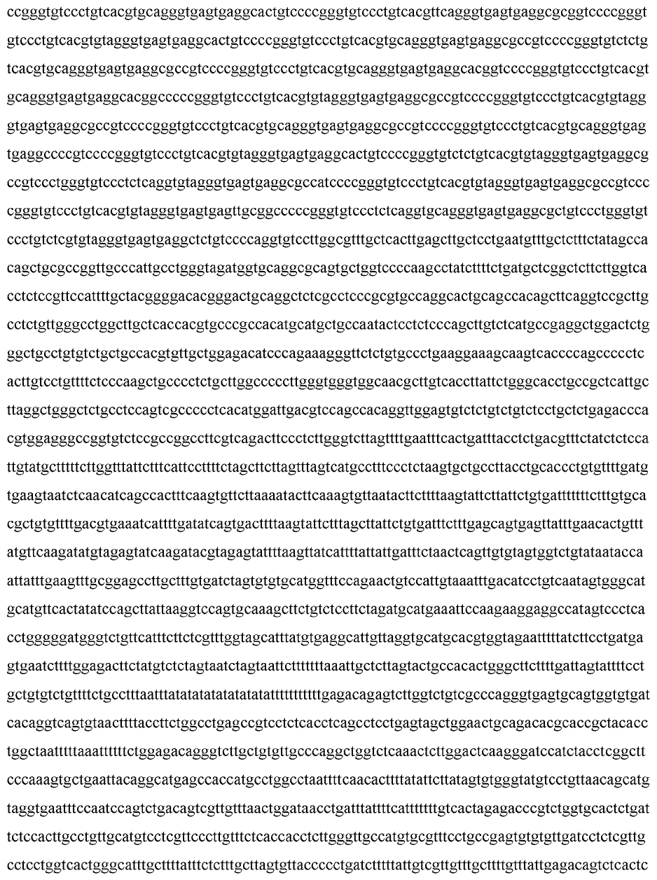

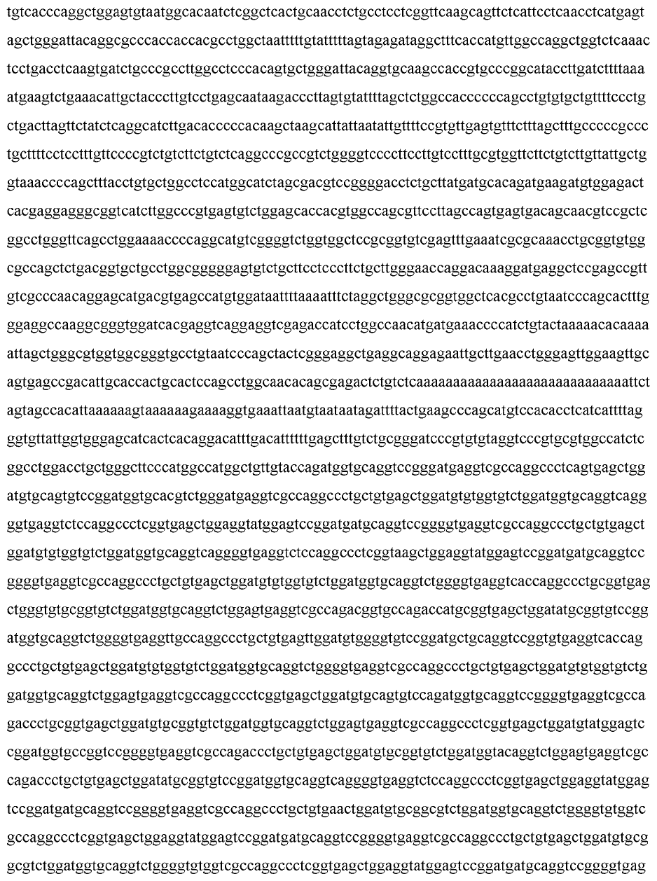

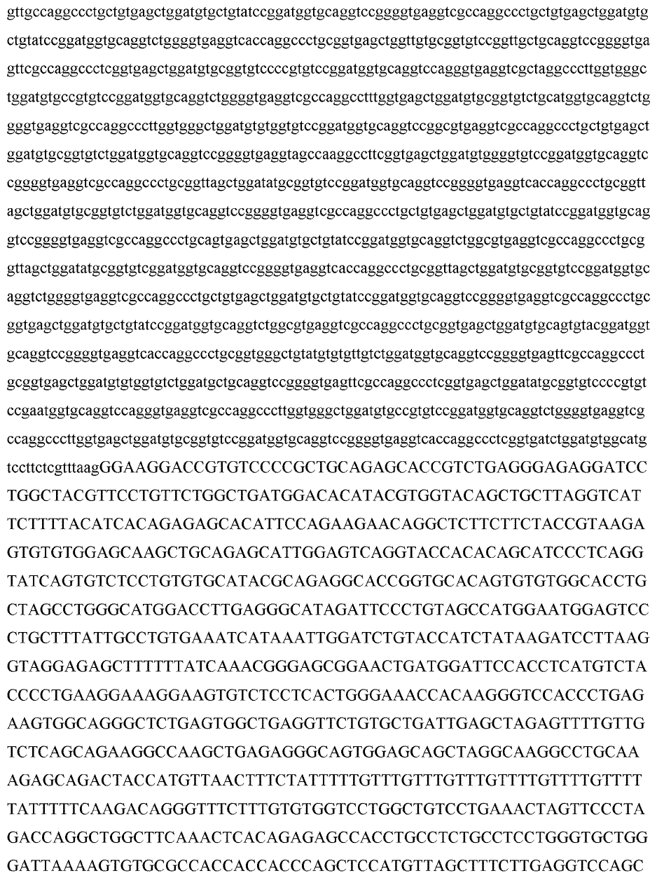

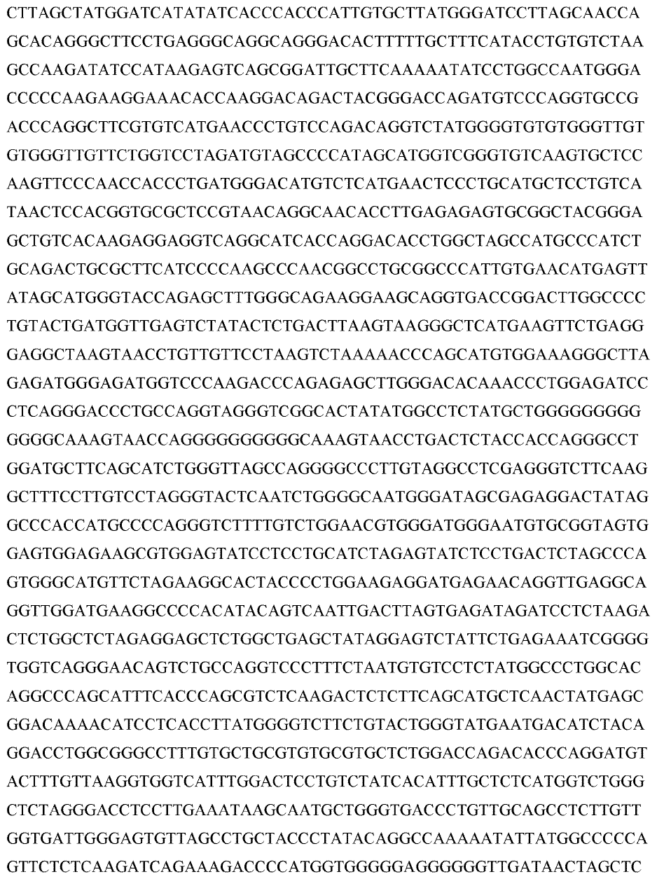

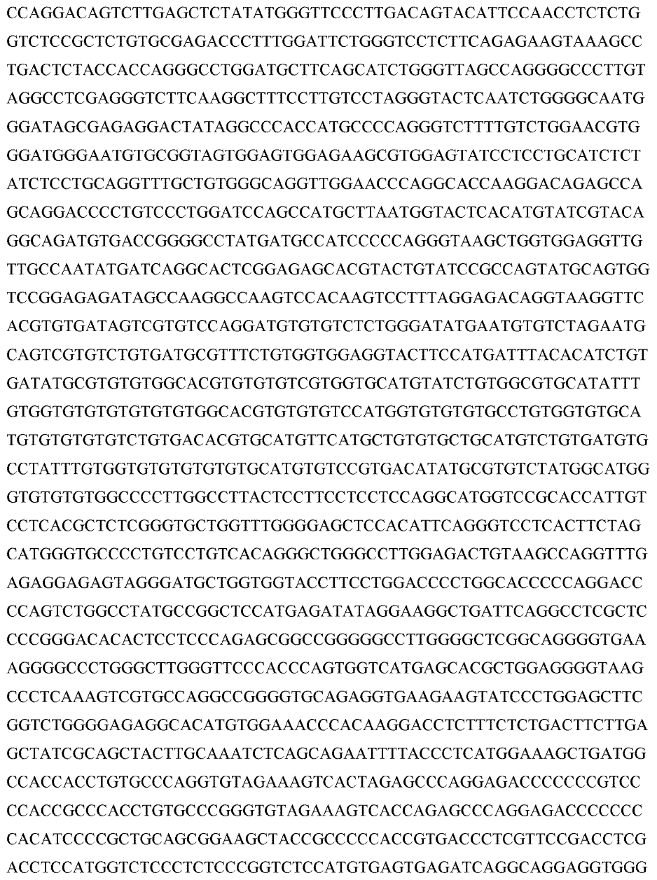

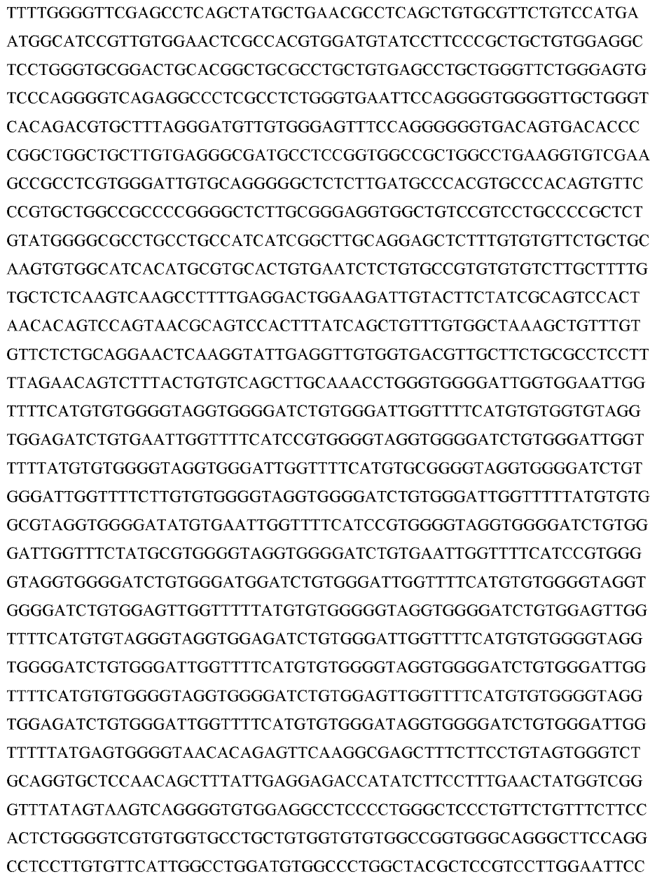

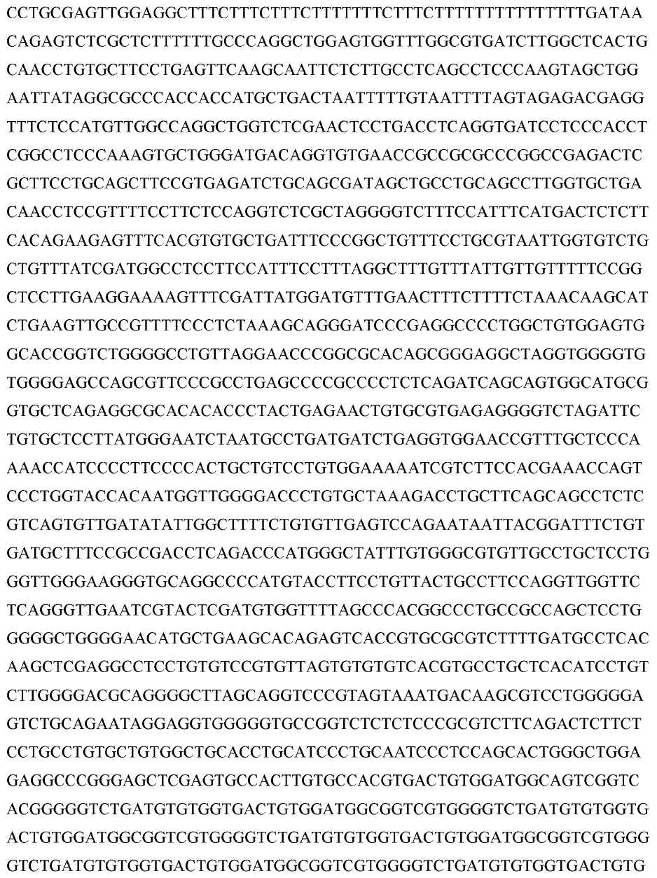

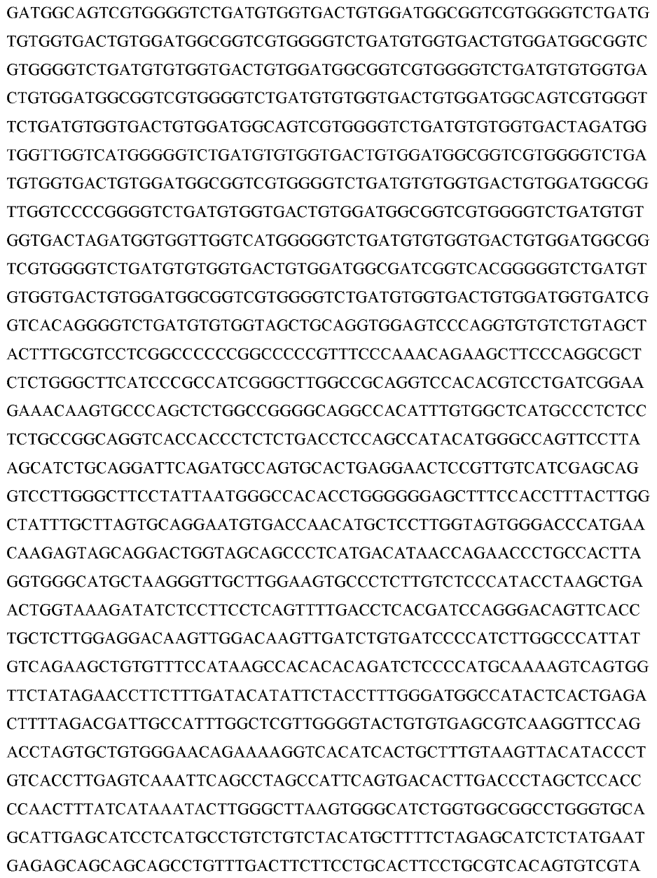

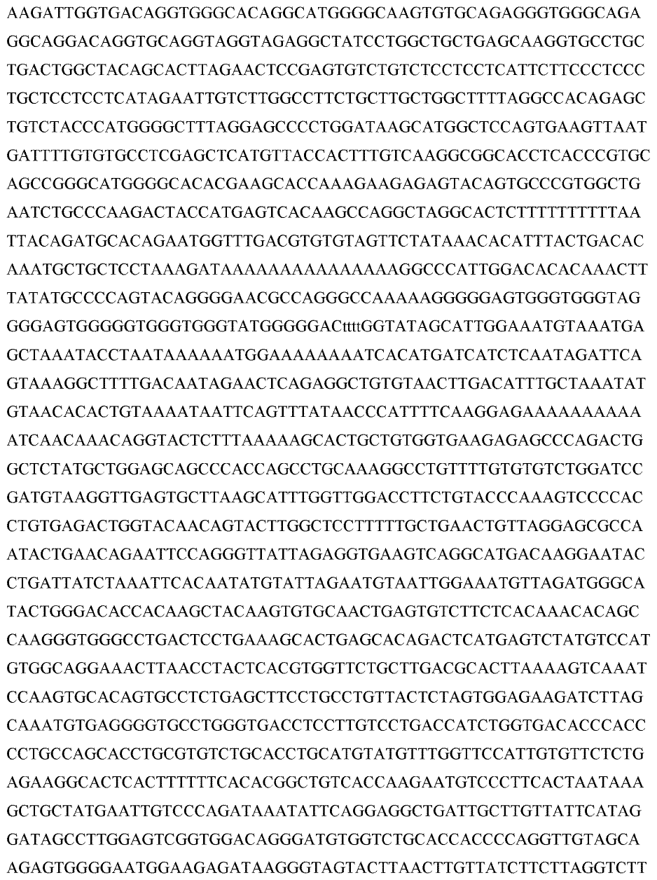

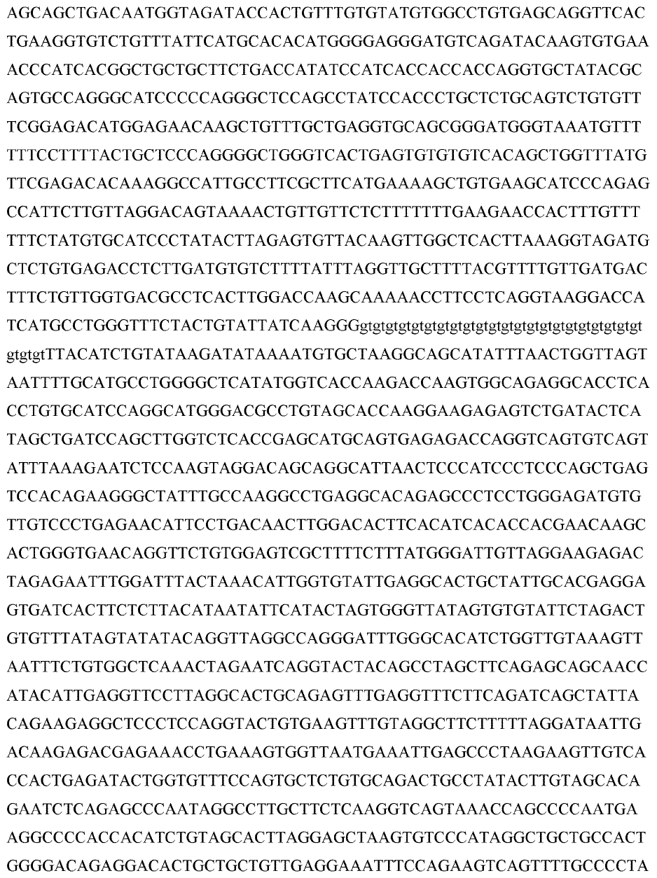

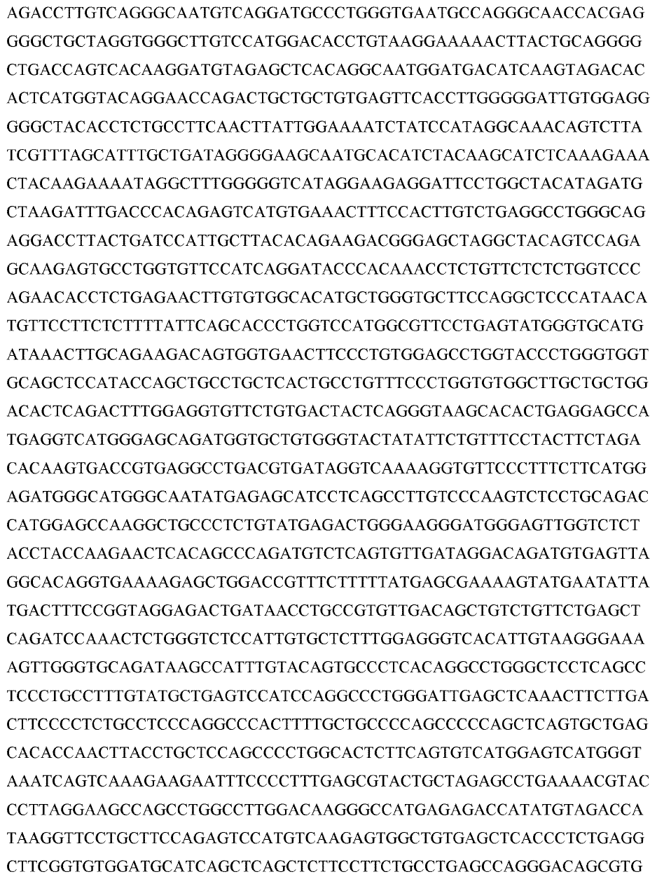

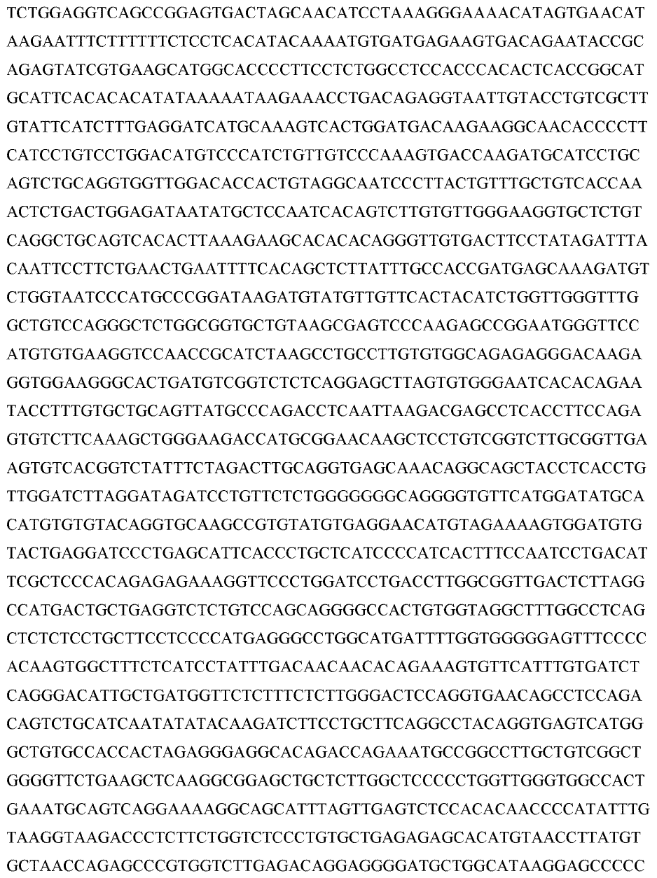

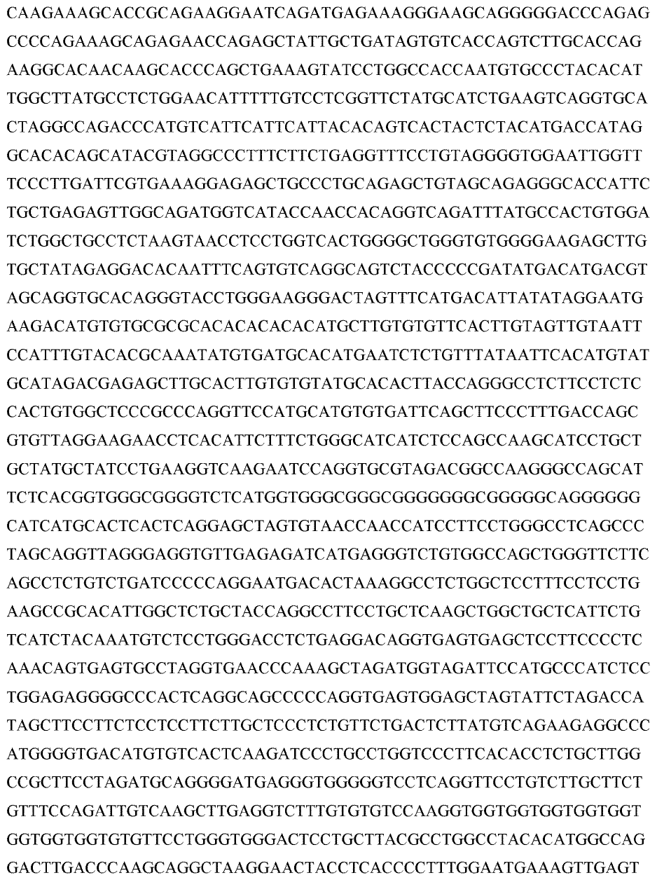

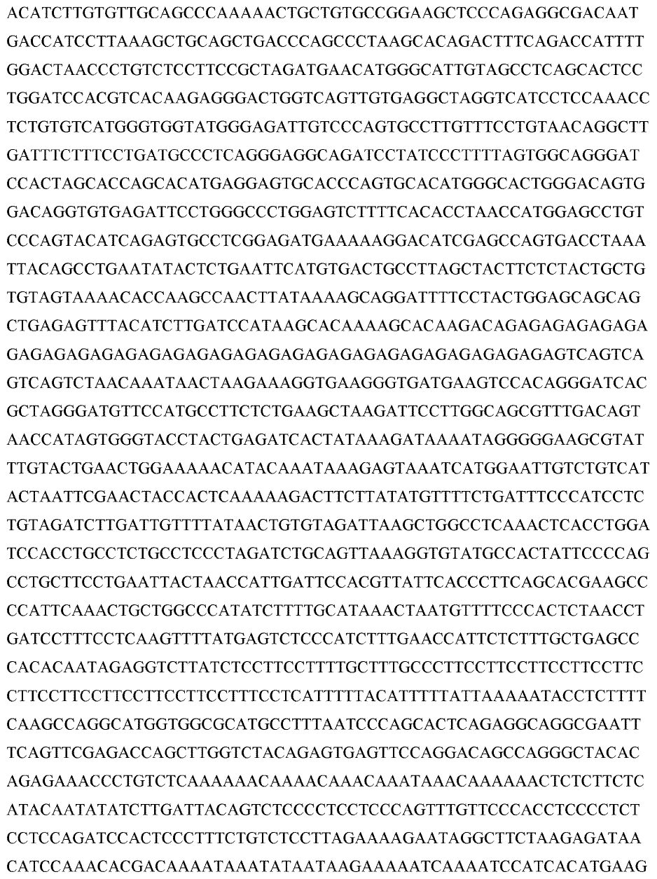

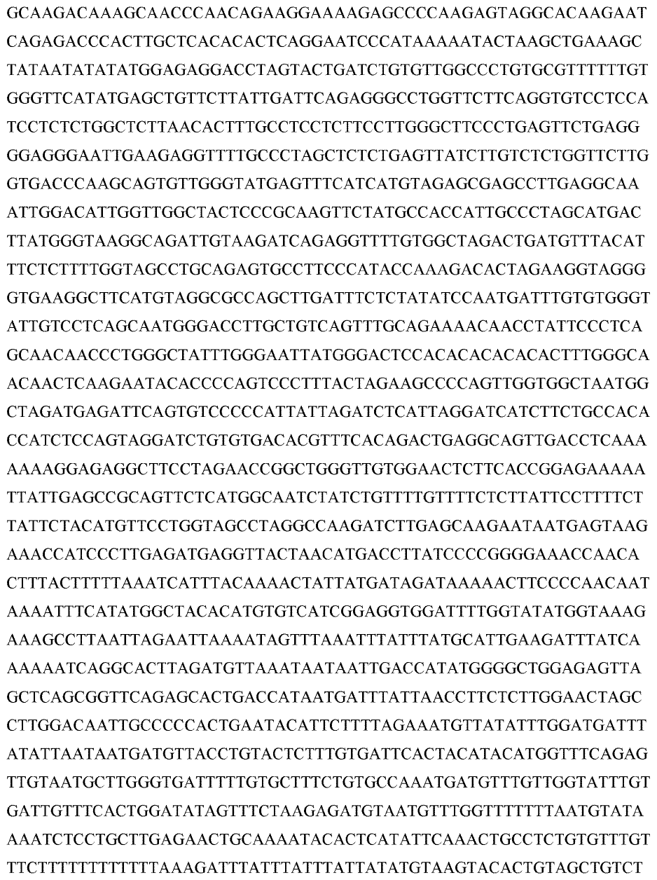

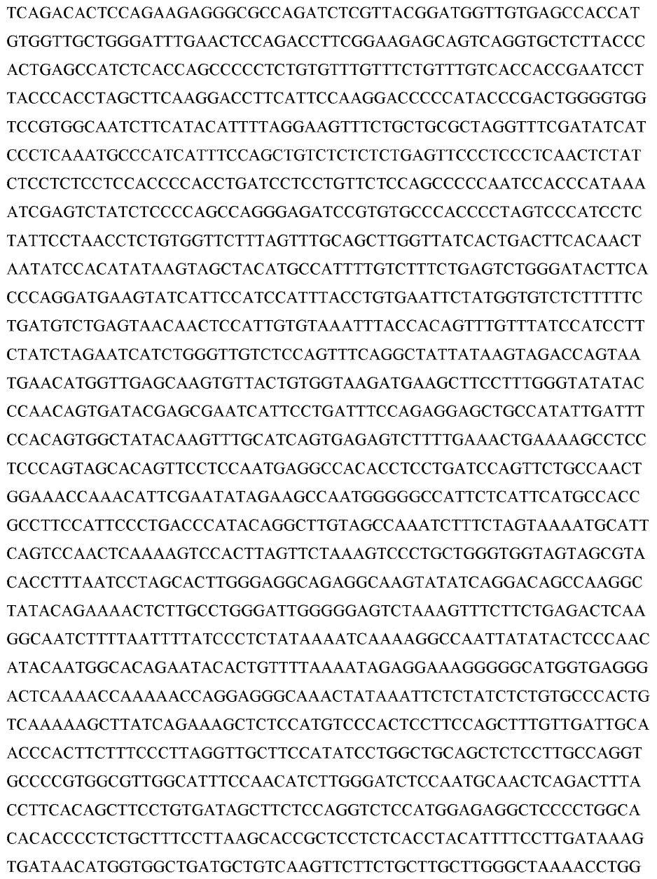

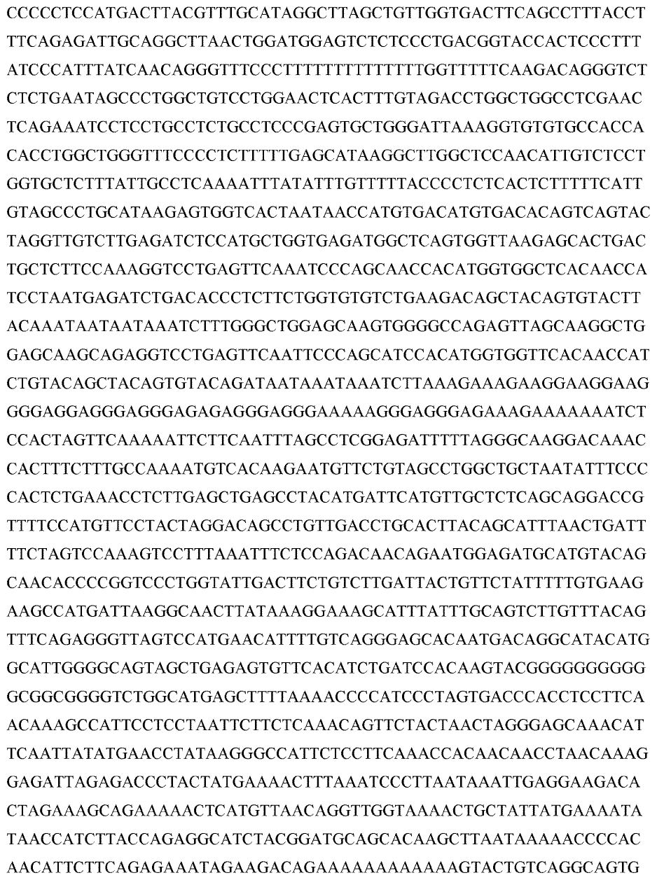

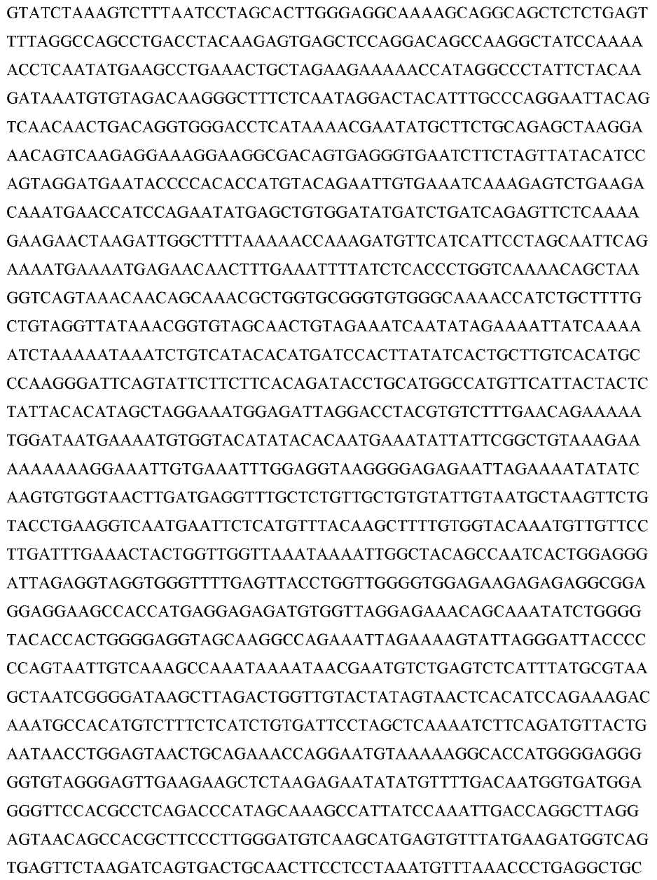

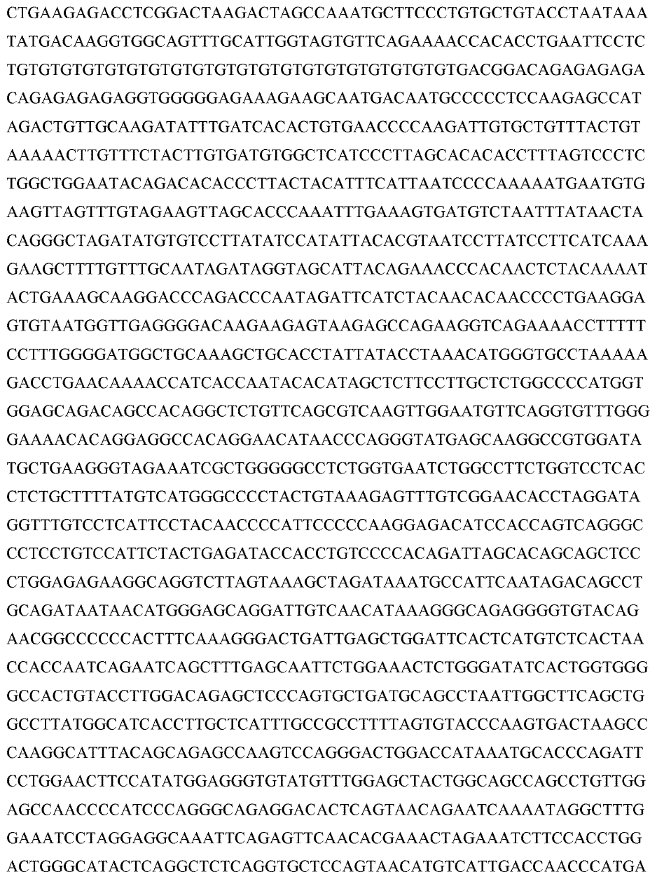

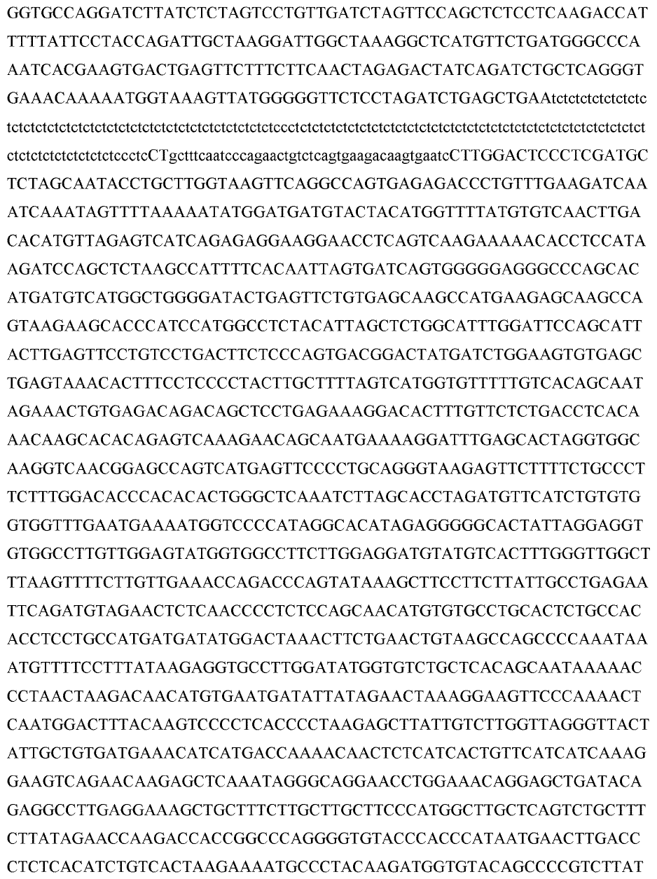

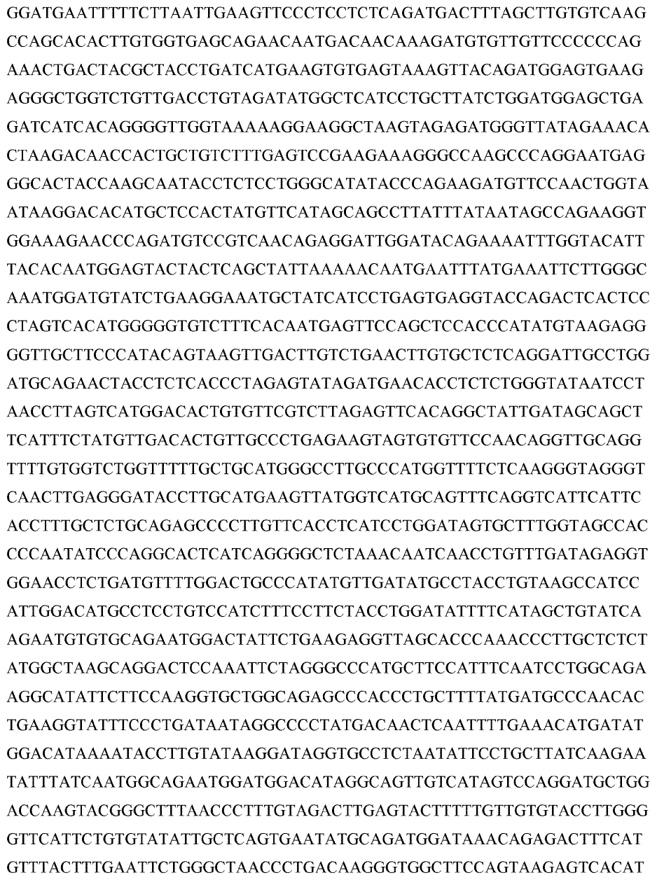

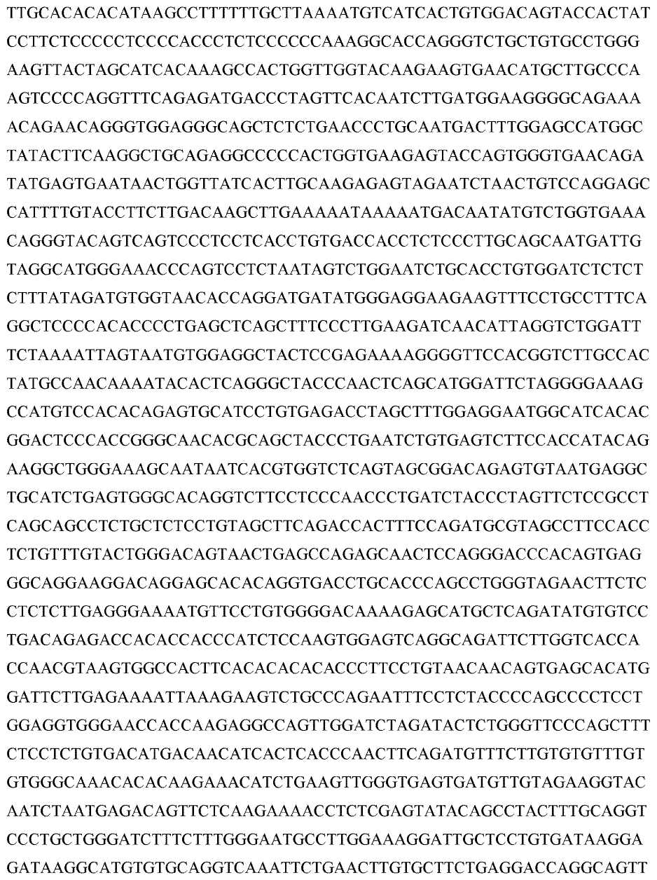

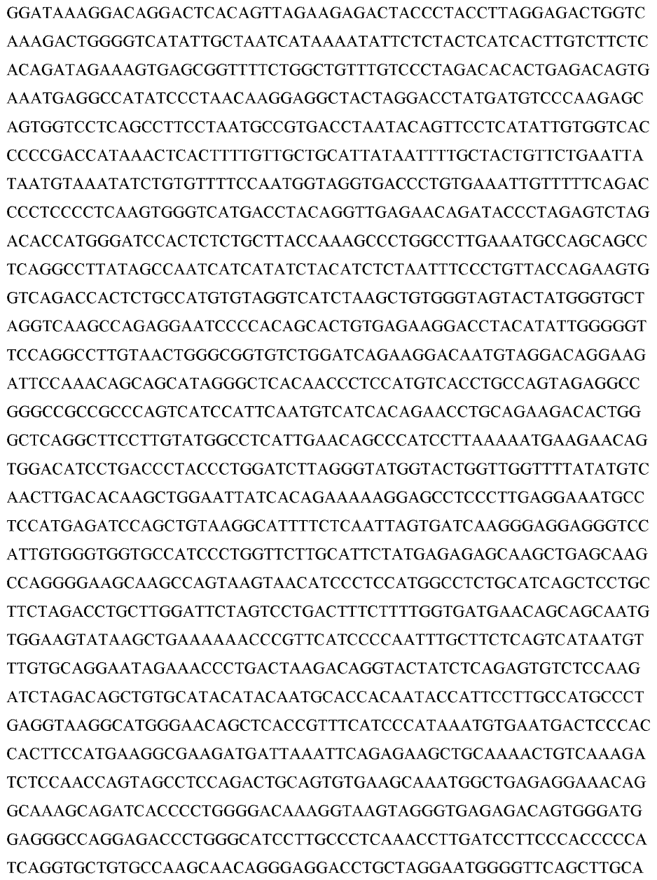

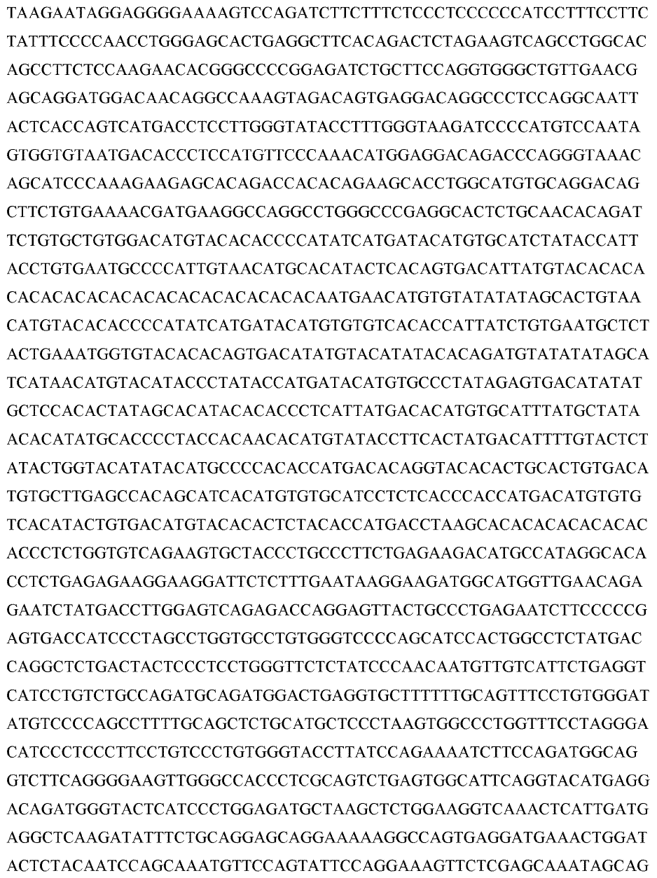

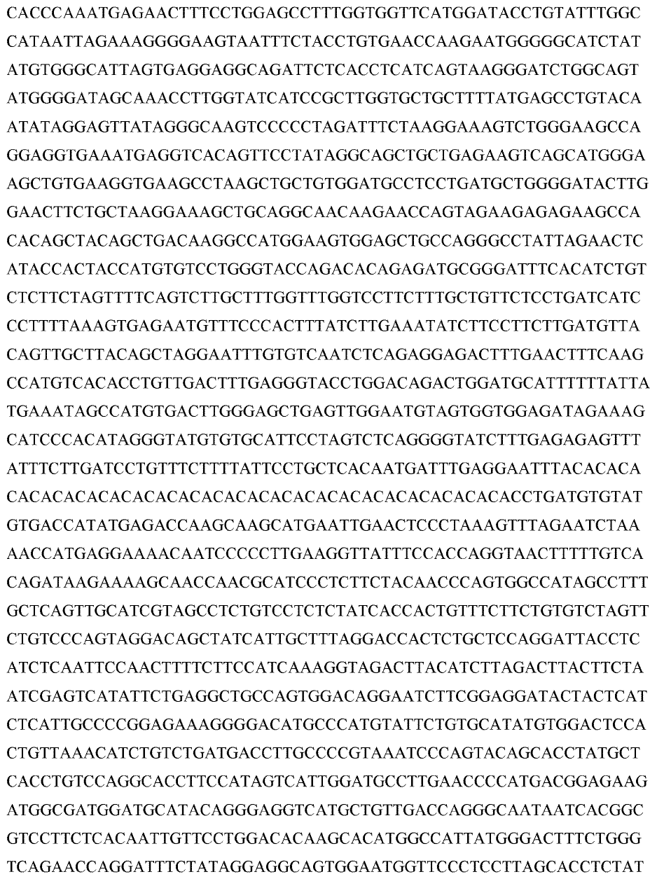

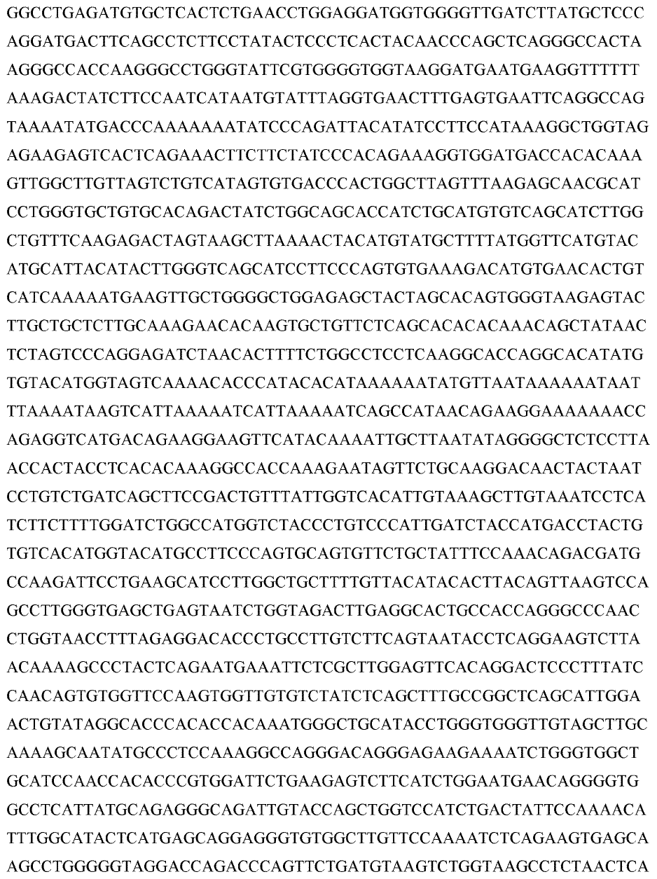

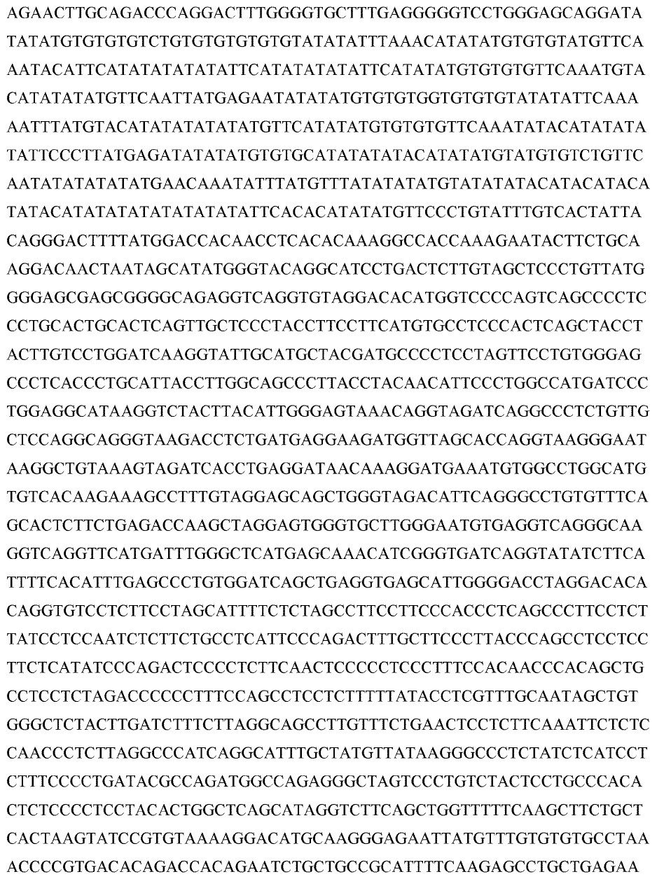

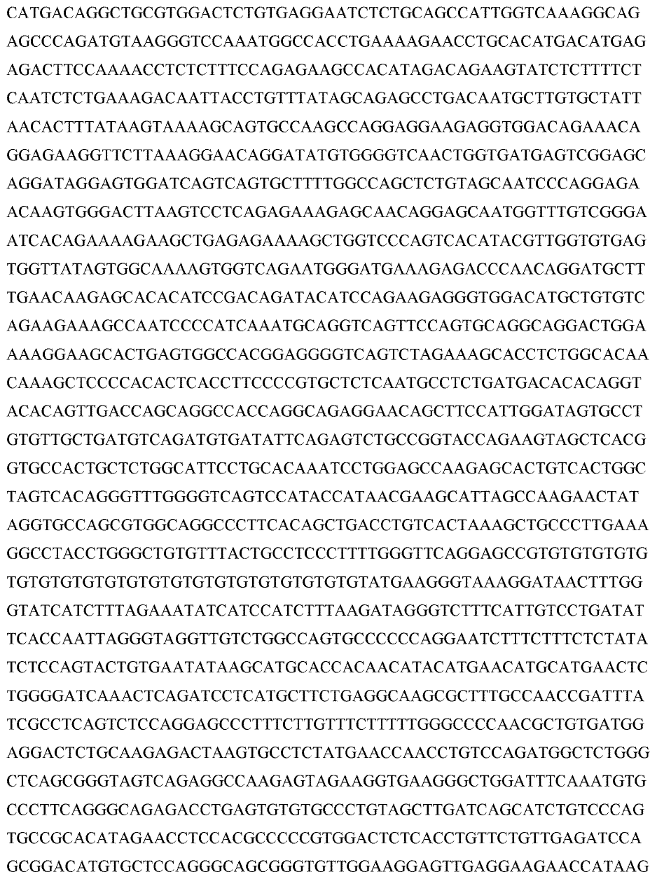

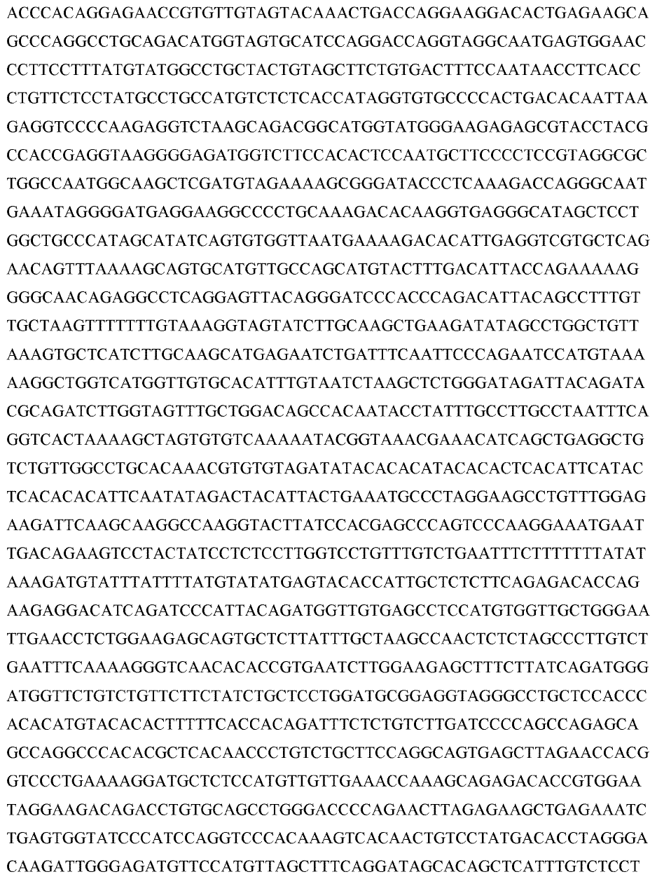

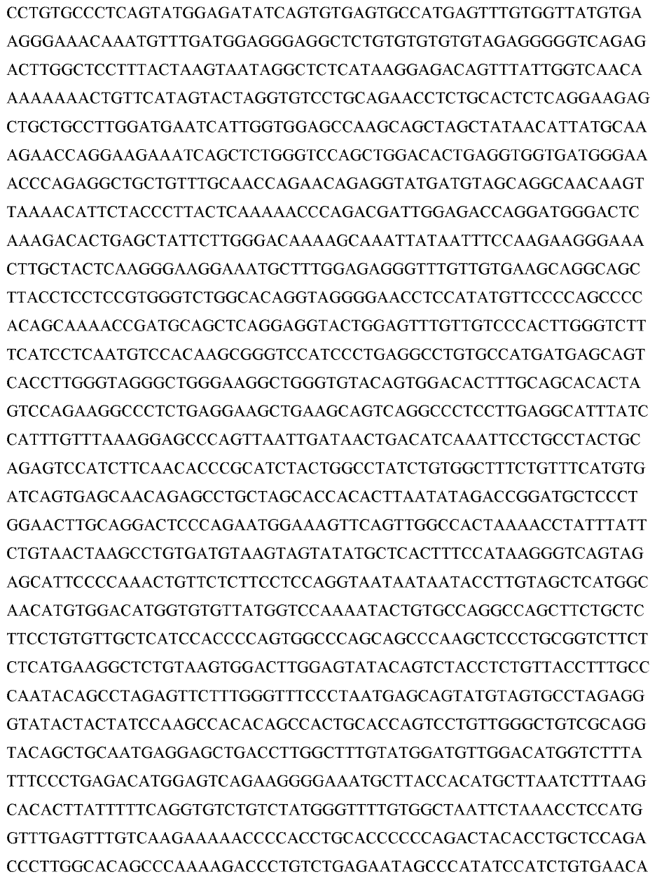

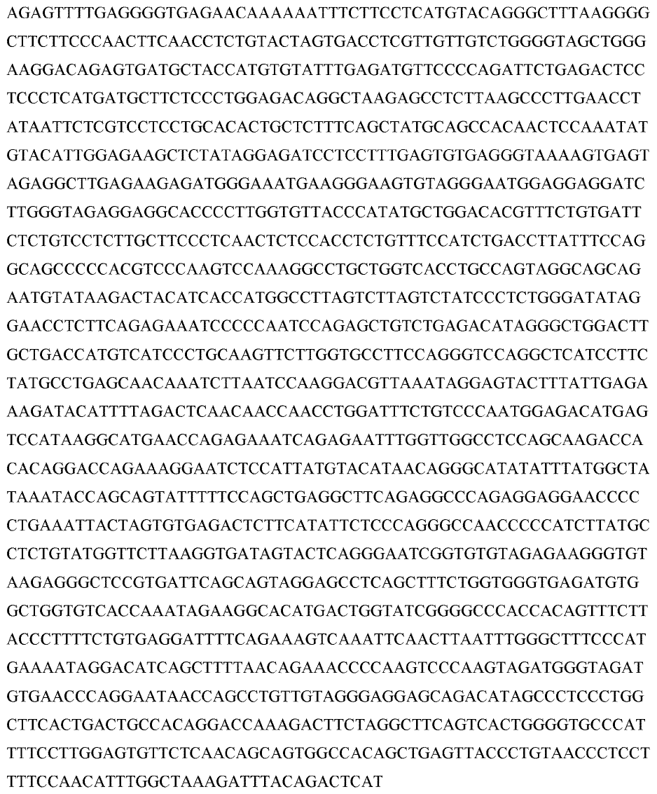

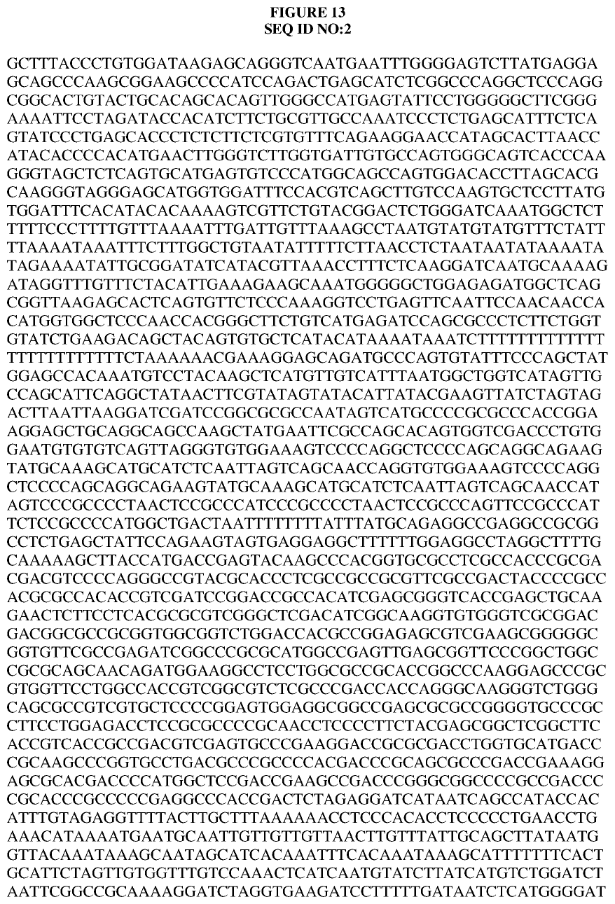

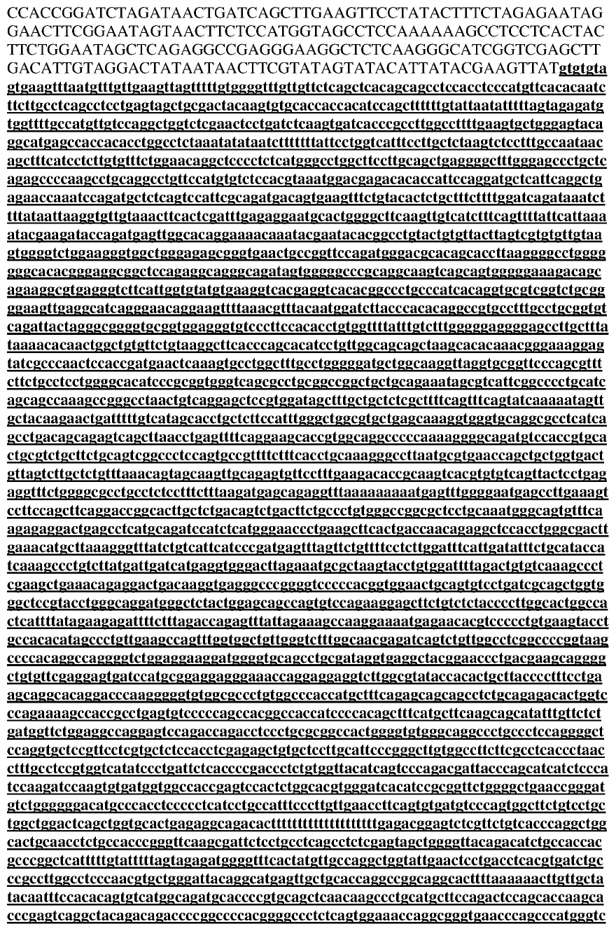

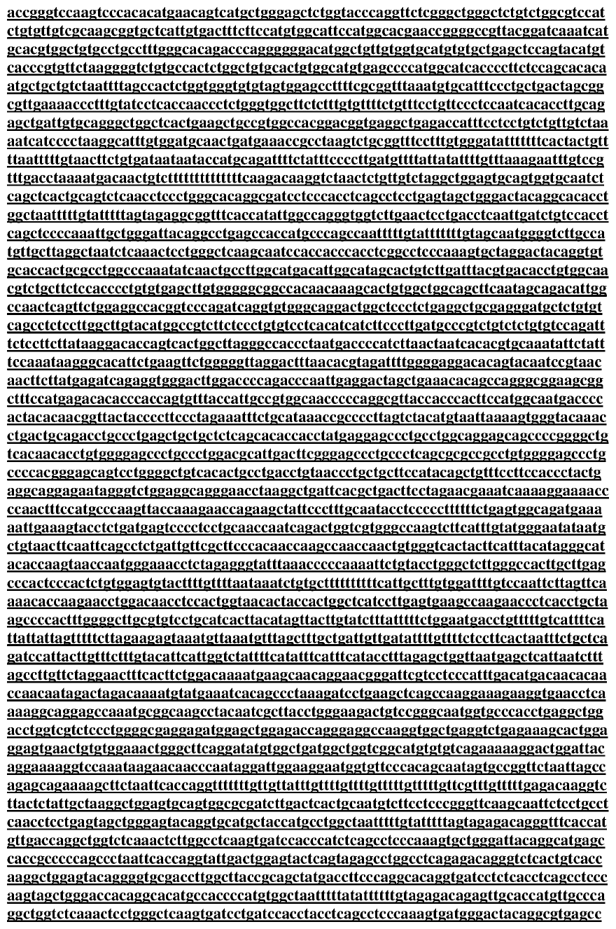

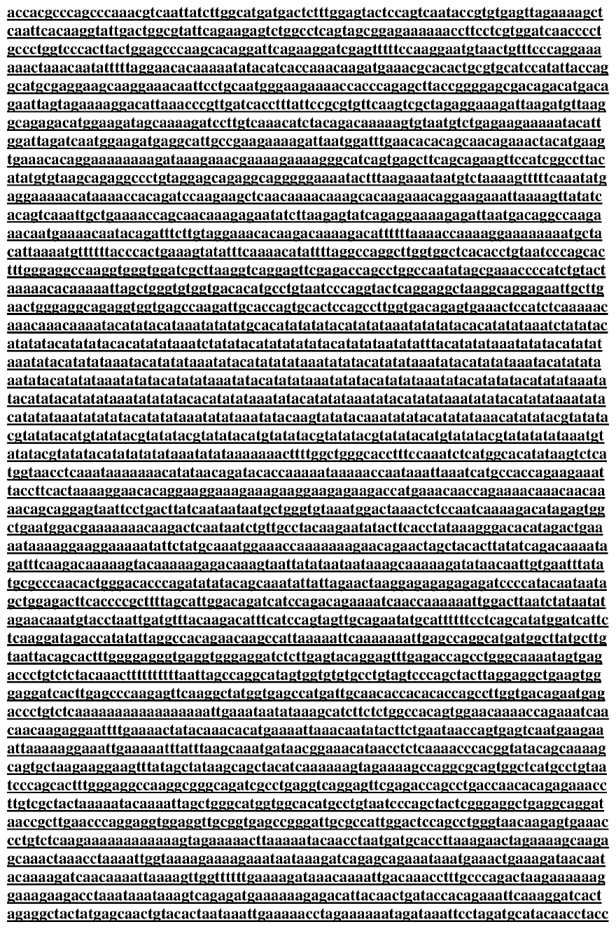

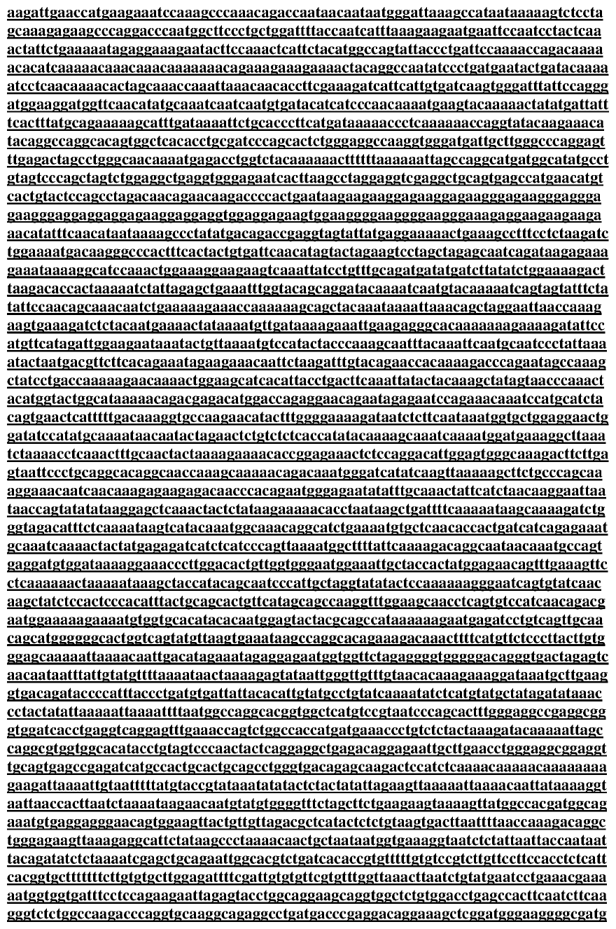

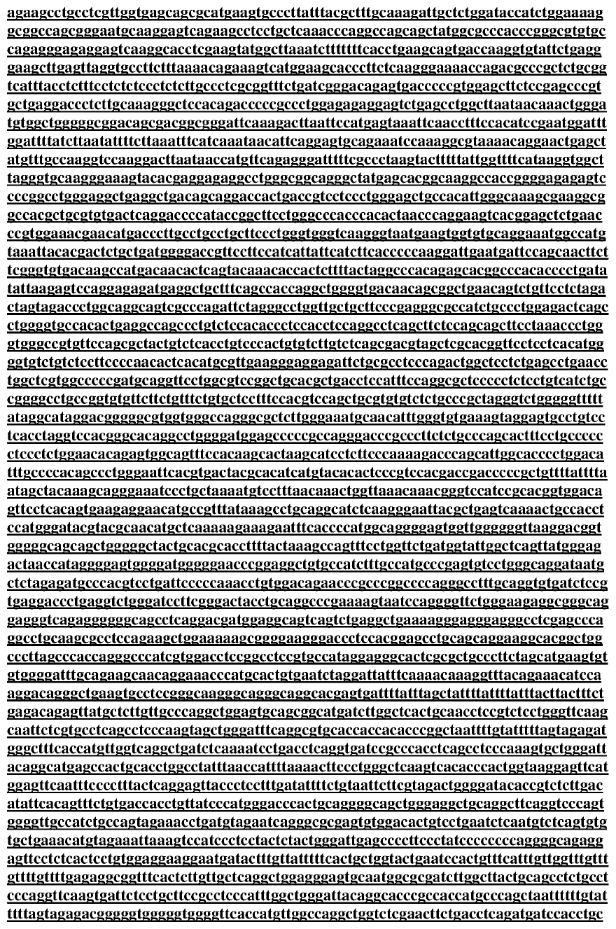

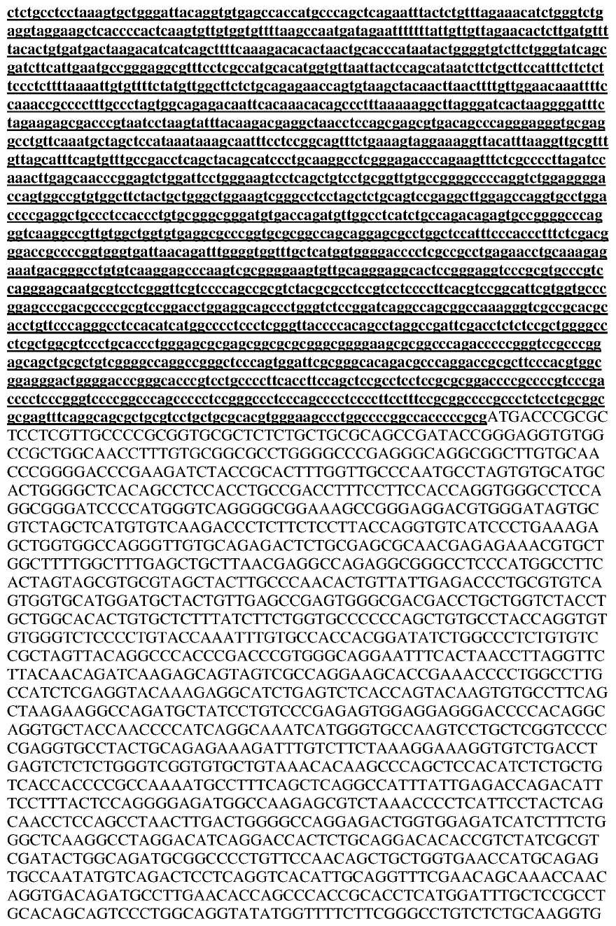

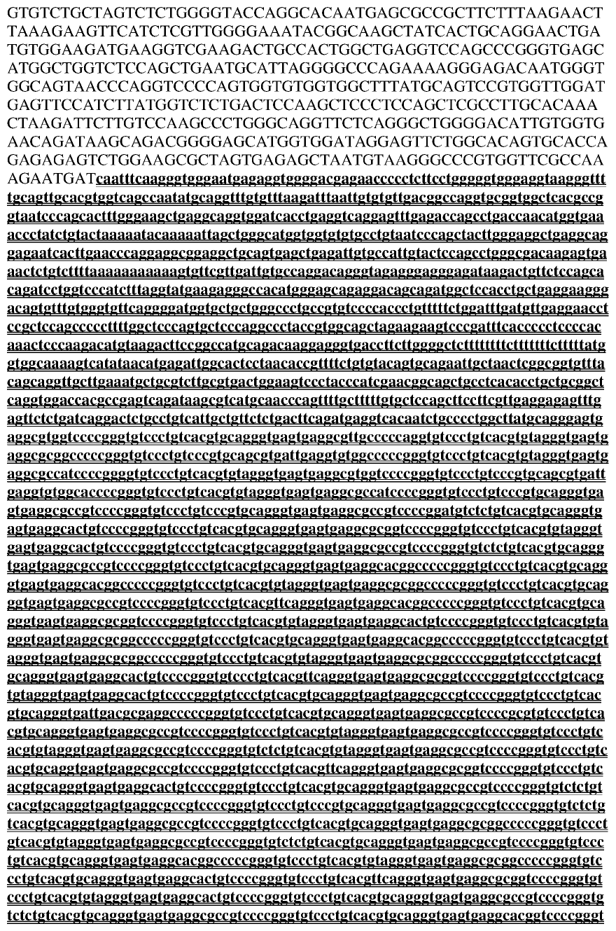

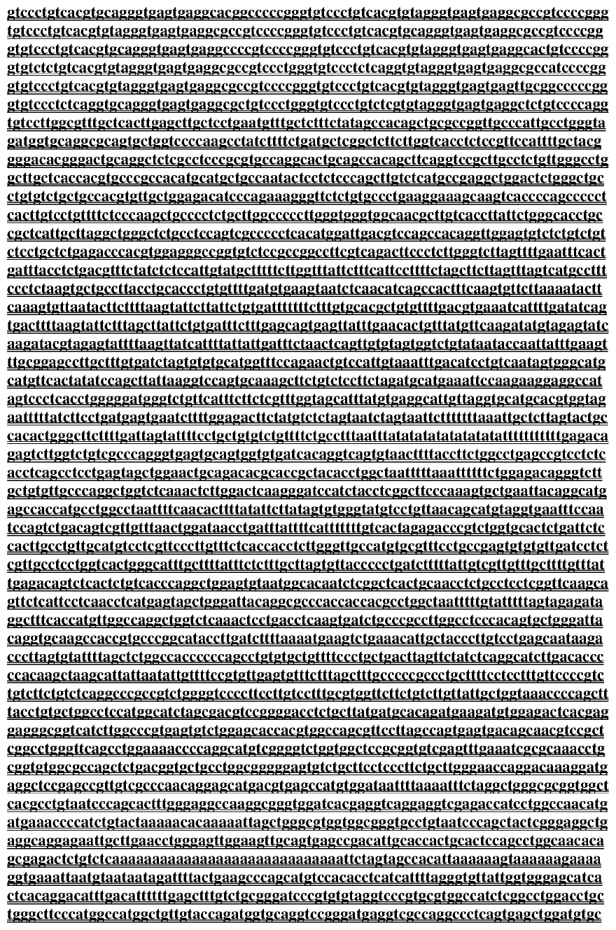

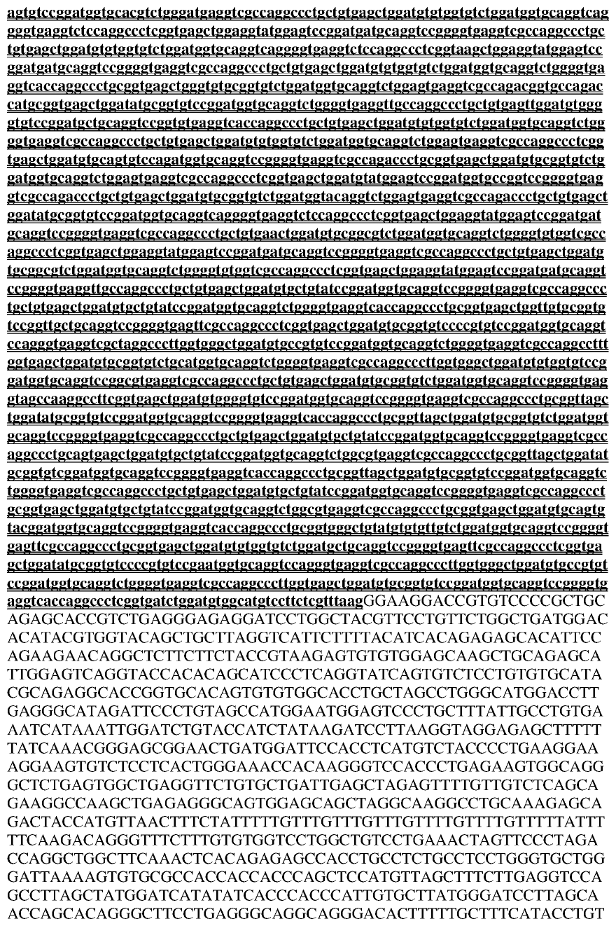

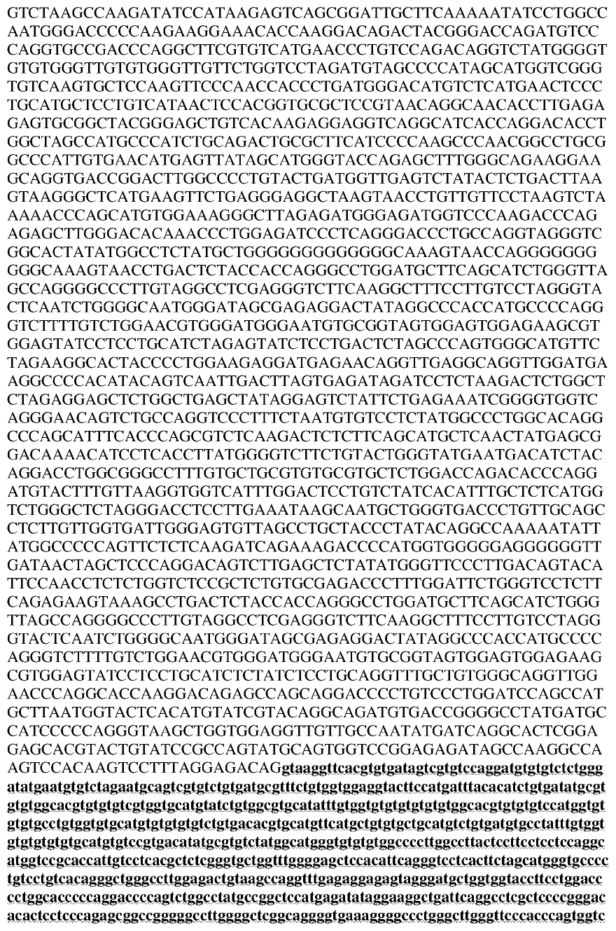

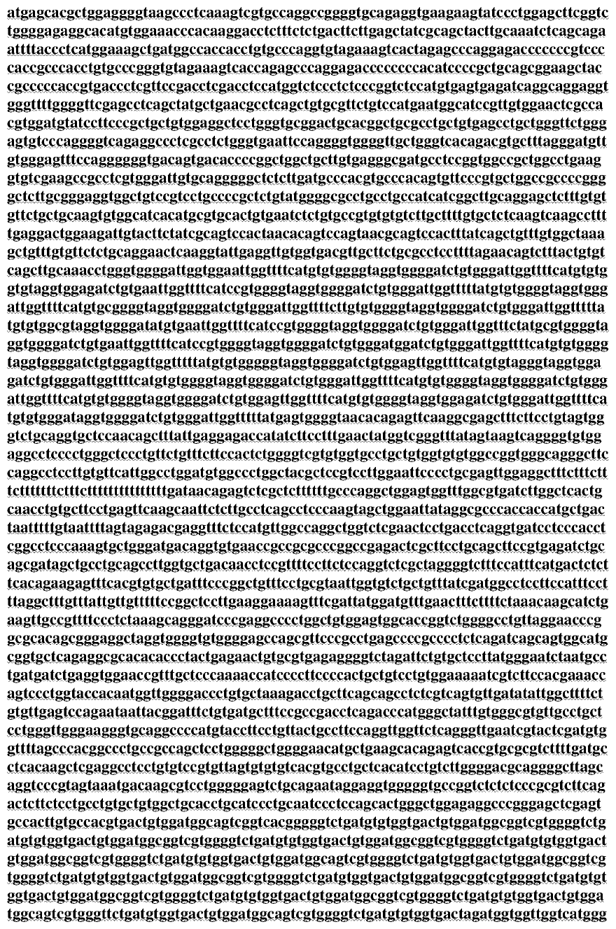

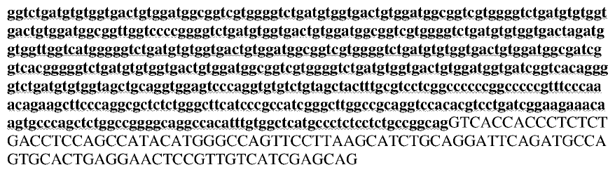

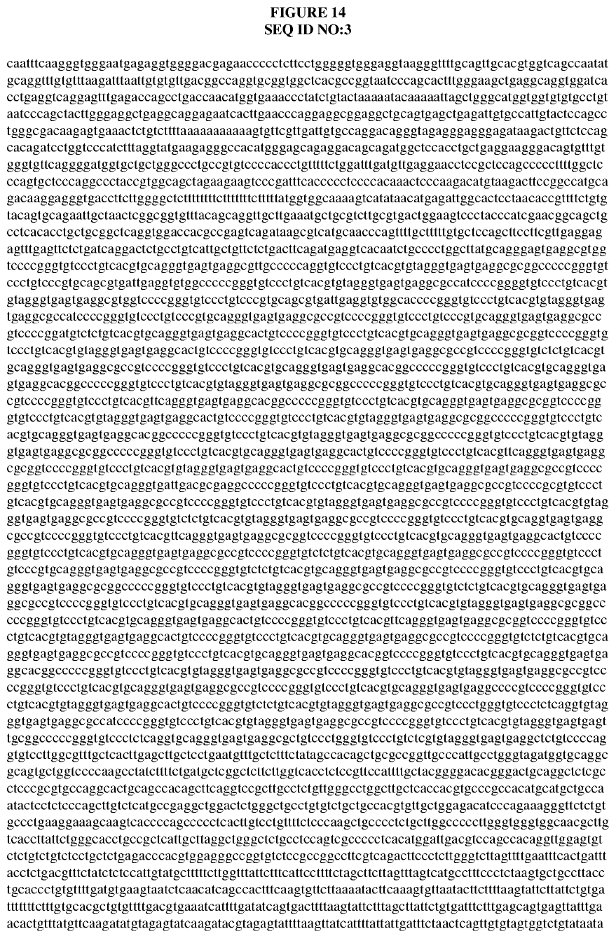

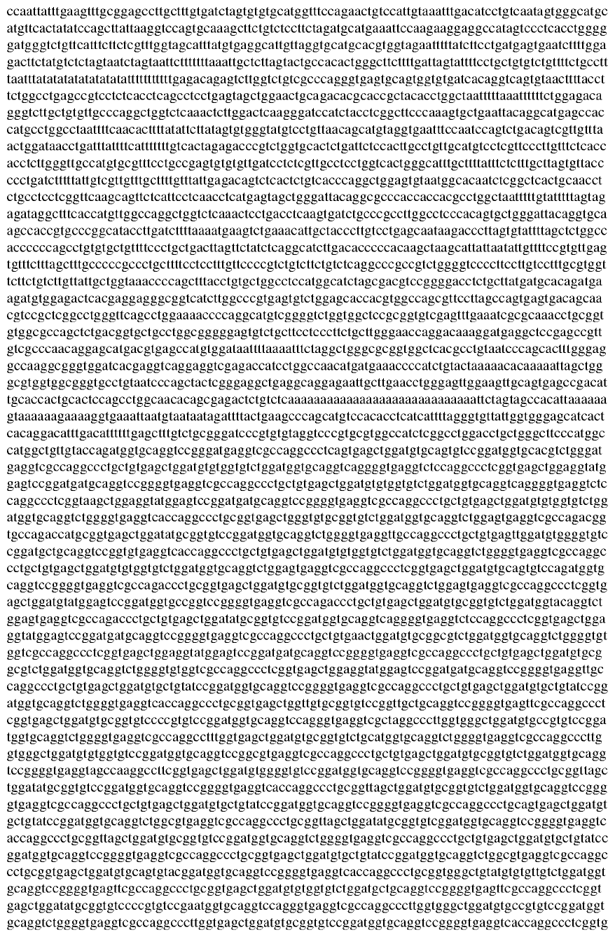

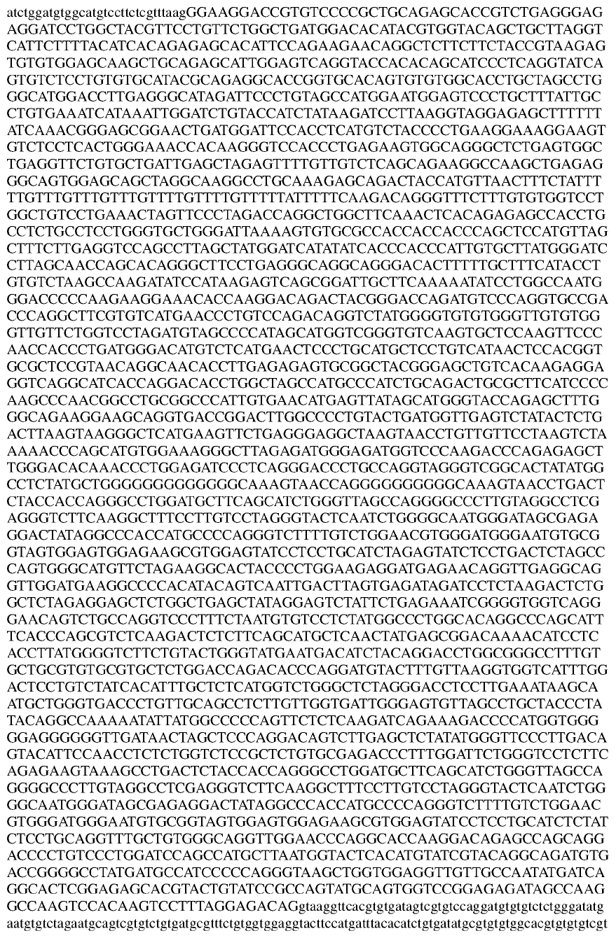

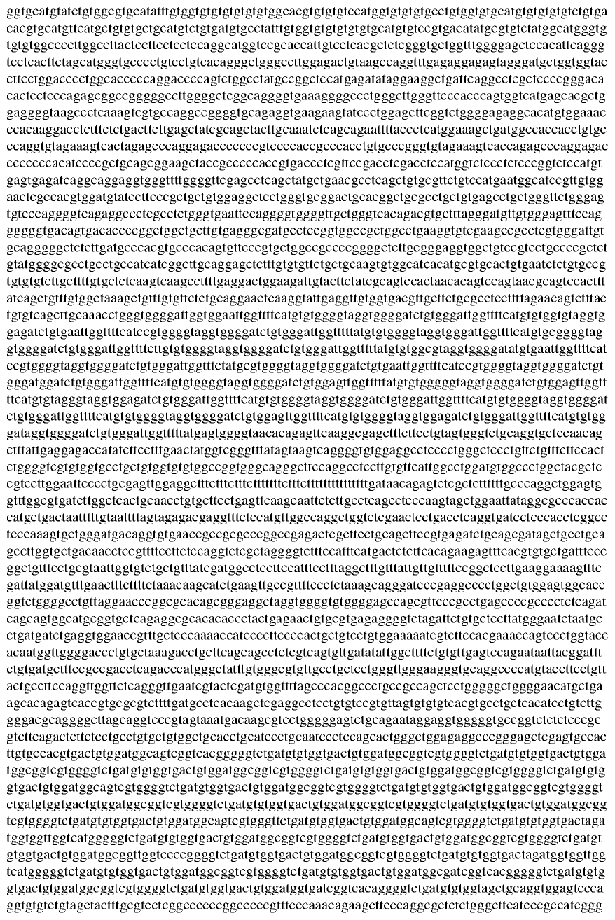

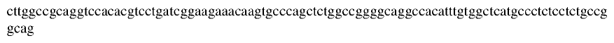

Ulcerative colitis is an inflammatory disease associated with telomere shortening and accelerated colon aging in human patients. G4.8 h Tert^{h/h} mice remained in good health, and their gastrointestinal tracts appeared normal. As depicted in FIG. 11E, cellular proliferation, assessed by EdU incorporation, in the colons of G4.8 h Tert^{h/h} mice showed a slight decrease, albeit statistically insignificant when compared to wildtype mice. To evaluate tissue renewal capacity under pathological conditions, mice were subjected to a 6-day dextran sodium sulfate (DSS) treatment to induce conditions akin to ulcerative colitis (FIG. 11A). DSS treatment led to similar colon shortening and spleen enlargement in both Tert^{+/+} and Tert^{h/h} mice, indicating DSS-induced comparable inflammatory responses in both groups (FIGS. 11B and 11C). The toxicity of DSS to colonic epithelial cells triggered a regenerative response upon toxin removal. In Tert^{h/h} mice, an average of about 2 EdU-positive cells per crypt cross-section were observed, significantly fewer than the average of 7 EdU-labeled cells per crypt cross-section in wildtype mice (FIGS. 11D and 11E). These results suggested that, while Tert^{h/h} mice with human-like short telomeres maintained tissue homeostasis during development and adulthood under normal physiological conditions, tissue renewal was more limited under pathological conditions due to their short telomeres and absence of telomerase activity.

1. A transgenic mouse comprising i) a background murine germline and ii) a humanized mouse telomerase (hmTert) gene contained in the background murine germline.

2. The transgenic mouse of claim 1, wherein the transgenic mouse is a humanized transgenic mouse.

3. The transgenic mouse of claim 1, wherein the hmTert gene comprises SEQ ID NO:2.

4. The transgenic mouse of claim 1, wherein the hmTert gene comprises SEQ ID NO:3.

5. The transgenic mouse of claim 1, wherein the background murine germline is from a species of Mus genus.

6. The transgenic mouse of claim 1, wherein the background murine germline is a strain of a Mus musculus line.

7. The transgenic mouse of claim 1, wherein the background murine germline is a substrain of C57BL/6 line.

8. The transgenic mouse of claim 1, wherein the transgenic mouse comprises expression maintenance of the hmTert gene.

9. The transgenic mouse of claim 1, wherein the transgenic mouse comprises humanized telomere homeostasis.

10. The transgenic mouse of claim 9, wherein the telomere homeostasis is maintained upon interbreeding for one or more generations.

11. The transgenic mouse of claim 1, wherein the mouse comprises one or more telomeres, and wherein the telomeres comprise an average telomere length from five to twenty (5-20) kilobase (kb).

12. (canceled)

13. A method for evaluating a treatment, the method comprising steps of i) administering the treatment to a transgenic mouse and ii) analyzing a response to the treatment in the transgenic mouse, wherein the response provides evaluation of the treatment.

14. The method of claim 13, wherein the treatment is a drug or a disease-causing agent.

15. (canceled)

16. The method of claim 13, wherein the response is an immune system response.

17. The method of claim 13, wherein the response is tumorigenesis.

18. The method of claim 13, wherein the response is senescence.

19. The method of claim 13, wherein the response is cellular aging.

20. The method of claim 13, wherein the response is to cancer.

21. The method of claim 13, wherein the response is to a rejuvenation or lifespan extension.

22. The method of claim 13, wherein the response is to an immunologic system reaction.

23. The method of claim 13, wherein the response is to a pharmaceutical event.

24. The method of claim 13, wherein the response is to an environmental exposure.

25. (canceled)

26. The method of claim 13, wherein the response is to a disease.

27. The method of claim 26, wherein the disease is an aging or age-related disease.

28. The method of claim 26, wherein the disease is a neurodegenerative disease.

29. The method of claim 26, wherein the disease is an immune system reaction.

30. A method of generating a transgenic mouse, the method comprising steps of:
inserting an hmTert gene into a background murine germline to form a modified embryonic stem cell; and
generating a first-generation mouse from the modified embryonic stem cell, wherein the first-generation mouse comprises the hmTert gene.

31. The method of claim 30, further comprising breeding one or more generations from the first-generation mouse to generate a progeny mouse.

32. The method of claim 31, wherein the breeding one or more generations comprises controlled breeding with one or more of hmTert, mTert, and mTert-knockout strains.

33. The method of claim 31, wherein the progeny mouse exhibits average telomere length of five to fifteen (5-15) kilobase.

34. (canceled)

35. (canceled)
